(19) United States
(12) Patent Application Publication
(10) Pub. No.: US 2012/0058088 A1
(43) Pub. Date: Mar. 8, 2012

(54) Resveratrol-Containing Compositions And Methods Of Use

(75) Inventor: William Sardi, La Verne, CA (US)

(73) Assignee: RESVERATROL PARTNERS, LLC, La Verne, CA (US)

(21) Appl. No.: 13/169,650

(22) Filed: Jun. 27, 2011

Related U.S. Application Data
(60) Provisional application No. 61/359,024, filed on Jun. 28, 2010.
(60) Provisional application No. 61/427,280, filed on Dec. 27, 2010.

(51) Int. Cl. A61K 31/05; A61K 31/352; A61K 31/19; A61K 31/59; A61K 31/728; A61K 31/726; A61K 35/34; A61K 35/30; A61K 35/44; A61P 9/00; A61P 35/00; A61P 27/02; A61P 25/28; A61P 29/00; A61K 31/6615

(52) U.S. Cl. 424/93.7; 514/733; 514/102; 514/456; 514/570; 514/167; 514/62

(57) ABSTRACT

A resveratrol-containing composition capable of providing a therapeutic benefit to a subject such as modulation of a biological activity, improving cell transplantation therapy, or improving macular degeneration or dystrophy treatments. The compositions comprise trans-resveratrol, a metal chelator, and one or more additional antioxidants such as phenolic antioxidants or vitamin D.

### CROSS-REFERENCE TO RELATED APPLICATIONS

This application claims priority under 35 U.S.C. §119(e) to U.S. Application Ser. Nos. 61/359,024 (filed on Jun. 28, 2010; pending) and 61/427,280 (filed on Dec. 27, 2010), both of which are herein incorporated by reference in their entirety.

### INCORPORATION OF TABLE

Table 3 in the present specification has been submitted as a separate electronic file, due to its large size, but should be understood as herein incorporated by reference in its entirety.

LENGTHY TABLES
The patent application contains a lengthy table section. A copy of the table is available in electronic form from the USPTO web site (http://seqdata.uspto.gov/?pageRequest=docDetail&DocID=US20120058088A1). An electronic copy of the table will also be available from the USPTO upon request and payment of the fee set forth in 37 CFR 1.19(b)(3).

### BACKGROUND

Despite a high level of risk factors such as cholesterol, diabetes, hypertension and a high intake of saturated fat, French males display the lowest mortality rate from ischaemic heart disease and cardiovascular diseases in Western industrialized nations (36% lower than the USA and 39% lower than the UK). The so-called ‘French Paradox’ (a low mortality rate specifically from cardiovascular diseases) may be due mainly to the regular consumption of wine (Renaud, S. et al. (1998) Novartis Found. Symp. 216:208-222, 152-158).

Resveratrol (3,4′,5-trihydroxy-trans-stilbene) is a naturally occurring phenolic compound found, for example in grape skins, that has been demonstrated to have beneficial properties relating to health of humans. In particular, resveratrol is believed to be beneficial to the functioning of the heart and in extending the life of human cells. Resveratrol, when used in dietary supplements, is generally produced as an alcohol extract from plant sources.

Calorie restricted diets have been shown to enhance survival and longevity by up-regulating survival/longevity genes or down-regulating genes whose expression enhances cellular damage. Mice have been used extensively as a model for genetic expression comparisons with humans. Without limitation, the validity of murine models to human gene expression reflects the fact that 98% of human and murine gene are homologous, and that mice and humans have about the same number of genes (e.g., approximately 30,000).

Despite the established benefits of a calorie restricted diet, the severity of the required dietary regime has limited adoption of this approach to increasing longevity. It would therefore be desirable to provide an alternative route to obtaining the benefits of calorie restriction that would avoid the need for dietary regulation and that would be amenable to widespread adoption. The present embodiments are directed to this and other needs.

### SUMMARY OF THE INVENTION

Embodiments of the present embodiments provide a composition that comprises trans-resveratrol, a metal chelating agent, and one or more additional antioxidants such as apigenin, caffeic acid, EGCG, ferulic acid, quercetin, or vitamin D, and methods of using the composition. The trans-resveratrol may be encapsulated to substantially preserve the biological activity of the composition from loss due to exposure of the trans-resveratrol to light or oxygen. Additional embodiments provide a method of protecting implanted stem cells by administering a composition that comprises trans-resveratrol, a metal chelating agent, and one or more additional antioxidants such as apigenin, caffeic acid, EGCG, ferulic acid, quercetin, or vitamin D in conjunction with or following stem cell implantation.

### BRIEF DESCRIPTION OF THE FIGURES

FIG. 1 shows the change in body weight of mice administered resveratrol or a composition of the present embodiments (Longevinex®) relative to control animals and animals maintained on a calorie restricted diet.

FIG. 2 shows the serum insulin level of mice administered resveratrol or a composition of the present embodiments (Longevinex®) relative to control animals and animals maintained on a calorie restricted diet.

FIG. 3 shows the serum glucose level of mice administered resveratrol (P=0.97) or a composition of the present embodiments (Longevinex®) (P=0.07) relative to control animals and animals maintained on a calorie restricted diet (P=0.10).

FIG. 4 shows a schematic of a mechanism of action that is consistent with the observed biological activities of the compositions of the present embodiments.

FIG. 5 is a bar graph showing the effects of resveratrol and a composition of the present embodiments (Longevinex®) on aortic flow in isolated perfused rat hearts.

FIG. 6 is a bar graph showing the effects of resveratrol and a composition of the present embodiments (Longevinex®) on coronary flow in isolated perfused rat hearts.

FIG. 7 is a bar graph showing the effects of resveratrol and a composition of the present embodiments (Longevinex®) on left ventricular developed pressure (LVDP) in isolated perfused rat hearts.

FIG. 8 is a bar graph showing the effects of resveratrol and a composition of the present embodiments (Longevinex®) on the maximum first derivative of left ventricular developed pressure (LV[dP/dt]ₘₐₓ) in isolated perfused rat hearts.

FIG. 9 is a bar graph showing the effects of resveratrol and a composition of the present embodiments (Longevinex®) on myocardial infarct size in isolated perfused rat hearts.

FIG. 10 is a bar graph showing the effects of resveratrol and a composition of the present embodiments (Longevinex®) on cardiomycyte apoptosis in isolated perfused rat hearts.

FIG. 11 is a chart showing the hormetic action of resveratrol, in which resveratrol dose [x-axis] is plotted against the values of cardiac function, infarct size, and apoptosis.

FIG. 12 is a bar graph showing the effects of 100 mg/kg resveratrol and a composition of the present embodiments (Longevinex®) on myocardial infarct size in isolated rabbit hearts.

FIGS. 13A through 13F are bar graphs comparing the effects of resveratrol and a composition of the present embodiments (Longevinex®) on aortic flow in isolated perfused rat hearts (FIG. 13A), coronary flow in isolated perfused rat hearts (FIG. 13B), on left ventricular developed pressure (LVDP) in isolated perfused rat hearts (FIG. 13C), on the maximum first derivative of left ventricular developed pressure (LV[dP/dt]ₘₐₓ) in isolated perfused rat hearts (FIG. 13D), on myocardial infarct size in isolated perfused rat hearts (FIG. 13E), and on cardiomycyte apoptosis in isolated perfused rat hearts (FIG. 13F).

FIGS. 14A and 14B are a Box Whisker plot (FIG. 14A) and a profile plot (FIG. 14B) comparing the effects of resveratrol and a composition of the present embodiments (Longevinex®) on global miRNA expression.

FIGS. 15A through 15C are a scatter plot (FIG. 15A), heatmap (FIG. 15B) and principal component analysis (FIG. 15C) of all samples, comparing the effects of resveratrol and a composition of the present embodiments (Longevinex®) on miRNA expression pattern.

FIGS. 16A and 16B are bar graphs comparing the effects of resveratrol and a composition of the present embodiments (Longevinex®) on phosphorylation of ERK1/2 (FIG. 16A) and p38 MAPK (FIG. 16B).

FIGS. 17A through 17C are bar graphs (top) quantifying the results of Western blots (bottom) depicting the regulation of miR-20b and the effects of antagomiR-20b on VEGF, Western blot analysis (FIG. 17A), Western blot analysis of samples pre-treated with antagomiR-20b (FIG. 17B), and a Taqman Real-time PCR quantification (FIG. 17C).

FIGS. 18A and 18B are bar graphs (top) quantifying the results of Western blots (bottom) depicting the regulation of miR-20b and the effects of antagomiR-20b on HIF-1a expression, including Western blot analysis (FIG. 18A) and Western blot analysis of samples when pre-treated with antagomiR-20b (FIG. 18B).

FIG. 19 is a bar graph comparing the intracellular quantification of reactive oxygen species for resveratrol and a composition of the present embodiments (Longevinex®).

### DETAILED DESCRIPTION

The present embodiments relate to a resveratrol-containing composition and especially a resveratrol-containing dietary composition (i.e., a composition amenable for oral ingestion by a recipient), and to methods of treatment and/or prophylaxis utilizing such compositions.

### A. Compositions of the Present Embodiments

In a preferred embodiment, the composition comprises or consists essentially of one or more plant extracts comprising trans-resveratrol, a metal chelating agent, and one or more additional antioxidants such as apigenin, caffeic acid, EGCG, ferulic acid, quercetin, or vitamin D. These compositions exhibit numerous benefits as compared to pure resveratrol alone. Preferred compositions comprise resveratrol (preferably, a composition dosage of from about 1 mg/kg of body weight to about 2 g/kg of body weight (more preferably from about 1 mg/kg of body weight to about 5 mg/kg of body weight), a chelator, and an antioxidant, and may also comprise other compounds such as emulsifiers, glycosaminoglycans, etc.

In a preferred embodiment, the composition is intended for a human, and comprises or consists essentially of trans-resveratrol in an amount of about 1.0 to about 5.0 mg/kg of body weight, preferably about 1.5 to about 2.5 mg/kg or about 3 to about 4.5 mg/kg of patient, and one or more of the following:
(a) a chelator such as phytic acid in an amount of about 0.5 to 1.5, 0.75 to 1.25 mg/kg, or about 1 mg/kg of patient;
(b) an additional phenolic antioxidants such as quercetin or ferulic acid in an amount of about 0.05 to 2, about 0.1 to 1.5, or about 0.15 to 1 mg/kg of patient, or both quercetin and ferulic acid in a total amount of about 0.15 to about 6, about 0.3 to 4.5, or about 0.45 to 3 mg/kg of patient; and
(c) an additional antioxidant such as Vitamin D in an amount of about 2.5 to 2500 or about 25 to 1250 micrograms/kg of patient.

In a preferred embodiment, the composition comprises resveratrol and is sold commercially as Longevinex® (Resveratrol Partners, LLC, San Dimas, Calif.). Four different formulations of Longevinex® have been sold, each consisting essentially of a plant extract comprising trans-resveratrol, quercetin dihydrate, and rice bran extract comprising phytic acid. Each dose of Longevinex® is suitable for administration to an average (e.g., 70 kg) human once daily. Each dose (e.g., a capsule) of the first generation Longevinex® composition consists essentially of: 5 mg Vitamin E (as mixed tocopherols), 215 mg of a mixture of Vitis vinifera (French red wine grape) and Polygonum cuspidatum (giant knotweed) extracts together comprising 100 mg of trans-resveratrol, 25 mg quercetin dihydrate, 75 mg rice bran extract comprising phytic acid, 380 mg rice bran oil comprising ferulic acid, and 55 mg sunflower lecithin. Each dose (e.g., a capsule) of the second generation Longevinex® composition consists essentially of: 215 mg of a mixture of Vitis vinifera (French red wine grape) and Polygonum cuspidatum (giant knotweed) extracts together comprising 100 mg of trans-resveratrol, 25 mg quercetin dihydrate, 75 mg rice bran extract comprising phytic acid, and 50 mg ferulate. Each dose (e.g., two capsules) of the third generation Longevinex® consists essentially of a Polygonum cuspidatum extract comprising 100 mg of trans-resveratrol, 1000 IU of cholecaliferol (Vitamin D3), quercetin, and rice bran extract comprising phytic acid. Each dose (e.g., two capsules) of the fourth generation Longevinex®, sold as Longevinex Advantage™, consists essentially of a Polygonum cuspidatum extract comprising 100 mg of trans-resveratrol, 1000 IU of cholecaliferol (Vitamin D3), grape seed extract, quercetin, ferulic acid, cocoa extract, lutein, green tea extract, rice bran extract comprising phytic acid, and hyaluronan.

1. Resveratrol

Resveratrol has been ascribed multiple beneficial biological effects (see, e.g., U.S. Pat. No. 7,345,178, which listing of disclosed effects is herein incorporated by reference), including preventing or treating cardiovascular disease, preventing or treating cancer, preventing or treating macular degeneration, attenuating or preventing diseases associated with aging, and other conditions and illnesses, including the incidence or severity of neurodegenerative diseases such as Alzheimer's Disease and Parkinson's Disease, and anti-inflammatory activity.

Resveratrol, also known as 3,4′,5 trihydroxystilbene, naturally exists in cis- and trans-stereoisomeric forms. Studies have shown that resveratrol is biologically active, providing several health benefits including cancer prevention, anti-inflammatory properties, and cardiovascular effects. To maintain biological activity for an “extended period” of time, the small molecules of plant or synthetic source preferably remain biologically active for time periods after which the molecules would naturally become biologically inactive due to degradation or molecular isomerization as a result of exposure to light, heat or oxygen. These destructive processes would likely occur during extraction, encapsulation or storage. For example, resveratrol possesses a half-life of approximately one day; consequently, it typically loses significant biological activity within two days of exposure to ambient conditions and during processing of dietary supplements. Preferably, the resveratrol used in the present compositions is entirely or primarily (e.g., more than 75, 80, 85, 90, or 95%) in the trans stereoisomeric form, i.e., trans-resveratrol.

Resveratrol may be synthesized chemically, or, more preferably, may be extracted from plant sources. Resveratrol is found in at least 72 species of plants distributed among 31 genera and 12 families. All of the families found to contain resveratrol belong to the spermatophytes division: Vitaceae, Myrtaceae, Dipterocarpaceae, Cyperaceae, Gnetaceae, Leguminosae, Pinaceae, Moraceae, Fagaceae, Liliaceae. Resveratrol has most often been reported in non-edible plants: vine, eucalyptus, spruce, and the tropical deciduous tree Bauhinia racemosa, Pterolobium Hexapetallum. Resveratrol is particularly found in grape skins and Giant Knotweed, cocoa and chocolate. Peanut sprouts are also a rich source of resveratrol.

In a preferred embodiment, the resveratrol is naturally derived, i.e., derived from at least one natural source such as plants (or parts thereof, such as tubers or fruit (including pulp and skins) from the plant). One preferred source is the seeds and/or skins of grapes, such as Vitis vinifera, Vitis labrusca, and Vitis rotundifolia. Another preferred source is Polygonum (Giant Knotweed) and, in particular, Polygonum cuspidatum (a species of giant knotweed). The natural derivation process includes those processes generally known in the art, including an extraction process in which a solvent is used to extract the small molecules from a natural source. The solvent includes aqueous solvents, organic solvents, and mixtures thereof. The solvent may include, but is not limited to, alcohols such as ethanol. By way of specific examples, the extracted material may include aqueous or organic solvent extracts of plants (or parts thereof), fruit juices (e.g., grape juice), and fermented liquors (e.g. wine) produced from plants or fruit juice, or mixtures of any of the foregoing. The extracted material may further include inert plant material naturally removed during the extraction process. The extracted material may be processed (physically and/or chemically) to remove the solvent and increase the concentration of the small molecules. For example, the solvent may be removed from the extract (e.g., by drying), leaving a dried powder.

In a preferred embodiment, the compositions comprise or consist essentially of a plant extract comprising trans-resveratrol, for example, a plant (grape) extract from Vitis vinifera, Vitis labrusca, or Vitis rotundifolia, a plant extract from a Polygonum species, or a combination of grape and/or Polygonum extracts. In a preferred embodiment, the compositions comprise or consist essentially of a mixture of grape and Polygonum extracts, each comprising trans-resveratrol. As used herein, the term “extract” or “plant extract” has its ordinary meaning of a concentrated pharmaceutical preparation of a plant obtained by removing active constituents (such as trans-resveratrol) with a suitable solvent or menstruum, which is evaporated away or otherwise removed to yield a residual mass of plant extract. The extract may be adjusted to a prescribed standard. Thus, it is understood by those skilled in the art that an “extract” or “plant extract” is not simply a pure active ingredient or ingredients, but instead contains secondary material from the source plant, for example, depending on the source plant, organic and inorganic salts, organic bases and acids, saponins, polyphenols, tannins, sugars, polysaccharides, etc.

In a preferred embodiment, trans-resveratrol is present in the composition in an amount of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or 95 percent by weight, or is present in any range between any two of these amounts, e.g., between about 10 and 30%, in an amount lesser than or greater than any two of these amounts, e.g. lesser than 15% or greater than 75%, or in an amount lesser than or equal to, or greater than or equal to any two of these amounts, e.g., lesser than or equal to 15%. In a different preferred embodiment, the trans-resveratrol is present in the composition in an amount of about 5-50%, 7.5-45%, 10-40%, 12.5-35%, 15-30%, or 20-25% by weight. In another preferred embodiment, trans-resveratrol is present in the composition in an amount of about 5-30% or 10-20% by weight. In a different preferred embodiment, trans-resveratrol is present in the composition in an amount of about 10-35%, 12.5-30%, or 15-25%, or in an amount of about 15-35% or 20-30% by weight.

In a preferred embodiment, trans-resveratrol is present in the composition in an amount calculated to provide a dosage in milligrams trans-resveratrol per kilogram of the patient to whom the dosage will be administered, for example, in an amount of about 0.25, 0.5, 0.75, 1, 1.25, 1.5, 1.75, 2, 2.25, 2.5, 2.75, 3, 3.25, 3.5, 3.75, 4, 4.25, 4.5, 4.75 or 5 mg trans-resveratrol per kilogram of patient, which is equivalent to a dosage of about 17.5, 35, 52.5, 70, 87.5, 105, 122.5, 140, 157.5, 175, 192.5, 210, 227.5, 245, 262.5, 280, 297.5, 315, 332.5, or 350 mg trans-resveratrol for the typical 70 kg human patient. In another preferred embodiment, trans-resveratrol is present in the composition in an amount of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 mg trans-resveratrol per kilogram of patient, or about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200 mg trans-resveratrol per kilogram of patient. The trans-resveratrol may also be present in any range between any two of these amounts, e.g., between about 0.25 and 4 mg/kg or between about 26 and 33 mg/kg, in an amount lesser than any of these amounts, e.g., lesser than about 2.5 mg/kg or 50 mg/kg, in an amount lesser than or equal to any of these amounts, e.g., lesser than or equal to about 50 mg/kg, in an amount greater than any of these amounts, e.g., greater than about 1.25 mg/kg or 25 mg/kg, or in an amount greater than or equal to any of these amounts, e.g., greater than or equal to about 2.5 mg/kg or 100 mg/kg. In a preferred embodiment, trans-resveratrol is present in the composition in an amount of about 1.5 to about 2.5 mg/kg for a human patient, or about 3 to about 4.5 mg/kg for a human patient.

2. Chelators

As used herein the term “chelator” refers to an organic compound that bonds with and removes free metal ions from solution. Examples of suitable chelators include ethylenediaminetetraacetic acid (EDTA), histidine, antibiotic drugs of the tetracycline family, pyridoxal 2-chlorobenzoyl hydrazone, desferrioxamine, dexrazoxane, deferasirox, pyoverdine, pseudan, citrate, NDGA (nordihydroguaiaretic acid: 1,4-bis[3,4-dihydroxyphenyl]2,3-dimethylbutane), ferulic acid and phytic acid. Preferably, the compositions of the present embodiments will provide a composition dosage of chelator of from about 1 g to about 15 g, more preferably from about 2 g to about 12 g.

Phytic acid is a particularly preferred chelator for the purposes of the present embodiments. As used herein, the term “phytic acid” refers to inositol hexaphosphate ((2,3,4,5,6-pentaphosphonooxycyclohexyl) dihydrogen phosphate; also known as “IP6”). Phytic acid is found in substantial amounts in whole grains, cereals, legumes, nuts, and seeds, and is the primary energy source for the germinating plant. Phytic acid and its lower phosphorylated forms (such as IP3) are also found in most mammalian cells, where they assist in regulating a variety of important cellular functions. Phytic acid is preferably provided in the form of a rice bran extract comprising phytic acid. Phytic acid is reported to function as an antioxidant by chelating divalent cations such as copper and iron, thereby preventing the generation of reactive oxygen species responsible for cell injury and carcinogenesis. The preferred composition dosage of phytic acid (for example, as derived from rice bran as an extract) is in the range of 200-12,000 mg, more preferably about 250-2500 mg per day.

Phytic acid also is believed to reduce the availability of metallic minerals that serve as growth factors in tumor cells, and as an inhibitor of calcium cystallization. It is also believed to serve as a neutrophil priming and motility agent. Additionally, phytic acid has been found to be neuroprotective, and thus to attenuate the severity of conditions associated with neurodegenerative diseases (especially Parkinson's Disease, camptocormia, and Alzheimer's Disease). The components of the present compositions are believed to enhance such neuroprotection.

The chelator may be of natural or synthetic source and may include, but not be limited to synthetic chelators such as desferrioxamine, EDTA, and d-penicillamine, or natural chelators such as lactoferrin, inositol hexaphosphate (IP6), quercetin, catechin, ferulic acid, curcumin, ellagic acid, hydroxytyrosol, anthocyanidin, etc.

In a preferred embodiment, a chelator is present in the composition in an amount of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or 95 percent by weight, or in an amount of about 0.25, 0.5, 0.75, 1, 1.25, 1.5, 1.75, 2, 2.25, 2.5, 2.75, 3, 3.25, 3.5, 3.75, 4, 4.25, 4.5, 4.75 or 5 mg chelator per kilogram of patient, or is present in any range between any two of these amounts, in an amount lesser than or greater than any two of these amounts, or in an amount lesser than or equal to, or greater than or equal to any two of these amounts. In a preferred embodiment, the chelator is present in the composition in an amount of about 10 to 35%, 15 to 30%, 20 to 30%, or 17.5 to 27.5%, or in amount of about 0.5 to 1.5 mg/kg of patient, 0.75 to 1.25 mg/kg of patient, or about 1 mg/kg of patient.

3. Additional Antioxidants

Additional antioxidants, for example phenolic antioxidants or Vitamin D may be added to the compositions. The additional phenolic antioxidants may be, for example, quercetin, ferulic acid, butein, fisetin, myricetin, kaempferol, cis-resveratrol or piceatannol. The antioxidants are believed to provide improved bioavailability of resveratrol by inhibiting resveratrol glucuronidation, and also act synergistically with resveratrol or independently of resveratrol to provide beneficial function.

The additional phenolic antioxidants may belong to a number of chemical classes of phenolic antioxidant compounds, such as the chalcones (e.g., butein), the flavonoids, the hydroxycinnamic acids, and the stilbenoids (e.g., cis-resveratrol, piceatannol). The flavonoids are a large class of phenolic compounds including the flavanols (2-phenyl-3,4-dihydro-2H-chromen-3-ols such as the catechins and epicatechins), the flavones (2-phenylchromen-4-ones such as apigenin), and the flavonols (3-hydroxy-2-phenylchromen-4-ones such as quercetin).

In one embodiment, the additional phenolic antioxidant comprises or consists of an antioxidant chalcone such as butein. In another embodiment, the additional phenolic antioxidant comprises or consists of a hydroxycinnamic acid selected from the group consisting of caffeic acid, cichoric acid, chlorogenic acid, caftaric acid, coumaric acid, coutaric acid, diferulic acids, fertaric acid, and ferulic acid, or combinations thereof. In a preferred embodiment, the additional phenolic antioxidant comprises or consists of a combination of caffeic acid and ferulic acid. In yet another embodiment, the additional phenolic antioxidant comprises or consists of a stilbenoid selected from the group consisting of cis-resveratrol and piceatannol.

In a further embodiment, the additional phenolic antioxidant comprises or consists of a flavanol selected from the group consisting of catechin (C), catechin 3-gallate (CG), epicatechin (EC), epicatechin 3-gallate (ECG), epigallocatechin (EGC), epigallocatechin 3-gallate (EGCG), gallocatechin (GC), and gallocatechin 3-gallate (GCG), or combinations thereof. In a preferred embodiment, the additional phenolic antioxidant comprises or consists of epigallocatechin 3-gallate (EGCG). In another embodiment, the additional phenolic antioxidant comprises or consists of a flavone selected from the group consisting of apigenin, baicalein, chrysin, diosmin, luteolin, scutellarein, tangeritin, and wogonin, or combinations thereof. In a preferred embodiment, the additional phenolic antioxidant comprises or consists of apigenin. In yet another embodiment, the additional phenolic antioxidant comprises or consists of a flavonol selected from the group consisting of quercetin, kaempferol, myricetin, fisetin, isorhamnetin, pachypodol, and rhamnazin, or combinations thereof. In a preferred embodiment, the additional phenolic antioxidant comprises or consists of quercetin.

The additional phenolic antioxidant may also comprise or consist of a combination of phenolic antioxidants, for example one or more flavonoids combined with one or hydroxycinnamic acids, etc. In one embodiment, the additional phenolic antioxidant comprises or consists of a combination of apigenin, caffeic acid, EGCG, ferulic acid, and quercetin.

In a preferred embodiment, one or more additional phenolic antioxidants are present in the composition in an amount of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or 95 percent by weight, or in an amount of about 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75 or 5 mg additional phenolic antioxidant per kilogram of patient, or is present in any range between any two of these amounts, in an amount lesser than or greater than any two of these amounts, or in an amount lesser than or equal to, or greater than or equal to any two of these amounts. In a preferred embodiment, the one or more additional phenolic antioxidants are present in the composition in an amount of about 1 to 25%, 2.5 to 20%, 5 to 15%, or 7.5 to 12.5%, or in an amount of about 5-10%, or in an amount of about 0.05 to 2, about 0.1 to 1.5, or about 0.15 to 1 mg/kg of patient, or in an amount of about 0.15 to about 6, about 0.3 to 4.5, or about 0.45 to 3 mg/kg of patient.

A non-phenolic antioxidant such as vitamin D may also be present in the compositions. As used herein, the term “Vitamin D” refers to a fat-soluble prohormone. Two major forms of vitamin D are vitamin D₂ (ergocalciferol) and vitamin D₃ (cholecalciferol) (DeLuca, H. F. et al. (1998) Nutr. Rev. 56:S4-S10). Vitamin D exhibits many biological actions. While vitamin D is widely known for its ability to stave off bone disease (rickets in growing children, osteoporosis in senior adults), it is becoming a central player in the battle against cancer. Regarding the role of vitamin D in immunity and cancer, vitamin D improves the chemotactic (affinity for) neutrophils to mobilize and migrate. Patients with rickets due to vitamin D deficiency are observed to have sluggish neutrophils that cannot migrate properly. Vitamin D stimulates the maturation of monocytes to macrophages. This results in an enlarged army of immune fighting cells to mount against tumors. Vitamin D is widely available commercially, and such preparations are suitable for the purposes of the present embodiments.

Vitamin D is essential for optimal muscle, bone, brain, immune and cardiovascular health and is undergoing re-discovery by aging researchers worldwide. Vitamin D supplementation up to 2000 IU has been shown to significantly reduce mortality rates, thus adding vitamin D to the lineup of molecules now considered to be true longevity factors (Autier, P. et al. (2007) Arch Intern Med. 167 (16):1730-1737). Its anti-calcifying properties (Zittermann, A. et al. (2007) Curr. Opin. Lipidology 18 (1):41-46) qualify vitamin D as another powerful agent that inhibits progressive overmineralization in the human body with advancing age and parallels the action of other mineral chelators in the compositions of the present embodiments. While the 1200 IU dose is three times more than the Recommended Daily Allowance, it is well within the Safe Upper Limit established by the National Academy of Sciences (2000 IU) and corresponds with a supplemental dosage recently found to be beneficial in a human clinical trial (Lappe, J. M. et al. (2007) Amer. J. Clin. Nutr. 85 (6):1586-1591). A 2,000 IU dosage is roughly equivalent the natural vitamin D3 produced by 15-30 minutes of total-body summer sun exposure at noontime at a southern latitude, for which no side effects have been reported. Preferably, the compositions of the present embodiments will provide a composition dosage of vitamin D of from about 100 IU to about 100,000 IU, more preferably from about 1,000 IU to about 50,000 IU.

Vitamin D3 works as an agent that mimics the response to a biological stressor, solar radiation. In particular, vitamin D3 upregulates protective genes involved in activation of the immune system, particularly neutrophil count and motility, and aids in overcoming the decline in endogenous vitamin D3 production with advancing age due to thickening of the skin, which reduces sun/skin production of vitamin D. Furthermore, vitamin D3 works synergistically to breakdown IP6 to IP3, thought to be a major active molecule. Resveratrol also works synergistically to sensitize cells to vitamin D3 (sensitizes the vitamin D receptor on the cell surface). Vitamin D serves to break down IP6 to IP3, which is its primary active form. Vitamin D is also believed to act as an immune system enhancing agent, boosting innate immunity in humans. In this capacity, vitamin D has been shown experimentally to have important cancer-preventive and cancer-curing properties. Resveratrol increases the sensitivity of the vitamin D receptor on the surface of cells, and thus is believed to act as an enhancing agent for vitamin D and as an anti-cancer agent. Resveratrol up-regulates the vitamin D receptor on the surface of healthy and cancer cells, and sensitizes cancer cells to vitamin D. Resveratrol is also believed to be a monoamine oxidase inhibitor (MAO Inhibitor).

In a preferred embodiment, Vitamin D is present in the composition in an amount of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or 95 percent by weight, or in an amount of about 0.25, 0.5, 0.75, 1, 1.25, 1.5, 1.75, 2, 2.25, 2.5, 2.75, 3, 3.25, 3.5, 3.75, 4, 4.25, 4.5, 4.75 or 5 micrograms (μg) Vitamin D per kilogram of patient, or in an amount of about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1600, 1700, 1800, 1900, 2000, 2100, 2200, 2300, 2400, 2500, 2600, 2700, 2800, 2900 or 3000 micrograms (μg) Vitamin D per kilogram of patient. Vitamin D may also be present in an amount of about 50-150,000 IU, about 100-100,000 IU, or about 1000 to 50,000 IU, where 1 microgram (μg) Vitamin D is equivalent to 40 IU. Vitamin D may also be present in any range between any two of these amounts, in an amount lesser than or greater than any two of these amounts, or in an amount lesser than or equal to, or greater than or equal to any two of these amounts. In a preferred embodiment, Vitamin D is present in the composition in an amount of about 2.5 to 2500 micrograms/kg of patient, or about 25 to 1250 micrograms/kg of patient.

4. Glycosaminoglycans

The compositions may comprise collagen-building nutrients (such as vitamin C-ascorbate, lysine, proline, etc.), and/or a glycosaminoglycan such as a shortened (low molecular weight) chain of hyaluronic acid (HA) or its singular components (glucosamine, glucuronate) or chondroitin sulfate, which are linear disaccharides (sugar-like molecules) that serve as structural components of cartilage, but in this combination serve as synergistic co-healing agents in non-cellular (connective) tissue that surrounds living cells. The collagen-building nutrients encourage the generation of collagen and small molecules that operate on intra-cellular basis.

As used herein, the term “hyaluronic acid” (also known as hyaluronan) refers to linear polymer composed of repeating disaccharides of D-glucuronic acid and D-N-acetylglucosamine, linked together via alternating β-1,4 and β-1,3 glycosidic bonds ([-β(1,4)-GlcUA-β (1,3)-GlcNAc-]ₙ). Hyaluronic acid can be 25,000 disaccharide repeats (n) in length. Hyaluronic acid is a water-retaining molecule that is generated naturally in the human body but in decreasing amounts as the body ages. Hyaluronic acid is a multifunctional glycosaminoglycan that forms the basis of the pericellular matrix of cells. Hyaluronic acid is synthesized by 3 different but related enzymes. U.S. Patent Application Publication 2004/0234497 discloses the use of hyaluronic acid for cancer drug delivery. The entire disclosure of that publication is incorporated herein by reference. Hyaluronic acid has been traditionally extracted from rooster combs, from bovine or fish vitreous humor, from microbial production or from other sources. Most preferably, the hyaluronic acid of the present embodiments is obtained from rooster combs. Hyaluronic acid is widely available commercially, and such preparations are suitable for the purposes of the present embodiments. Preferably, the compositions of the present embodiments will provide a composition dosage of hyaluronic acid of from about 1 mg to about 400 mg, more preferably from about 50 mg to about 200 mg.

Hyaluronic acid is the water gelling molecule of the human body which serves as its scaffolding and hydrating agent. As aging progresses, less hyaluronic acid is produced, resulting in wrinkled skin, thinning hair, unlubricated joints. The chelators of the present composition also help to preserve hyaluronic acid in the body. The hyaluronic acid component and the mineral chelating components (e.g., resveratrol, quercetin, phytic acid IP6, ferulate) work as a total anti-aging strategy to maintain youthful function within cells and connective tissues. Hyaluronic acid is believed to have an affinity to cancer cells. It is believed to serve as a delivery and targeting (drug delivery agent) molecule in blood circulation and to address aging of the connective tissue. The collapse and loss of integrity of connective tissue between cells provides the signs of aging (e.g., skin wrinkling, hair thinning, joint stiffness, loss of stature, etc.). The addition of hyaluronic acid to the present compositions is believed to activate fibroblast cells in the human body to produce additional hyaluronic acid, thus serving to preserve connective tissue (collagen) in a youthful state.

In a preferred embodiment, one or more glycosaminoglycans are present in the composition in an amount of about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or 95 percent by weight, or in an amount of about 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75 or 10 mg glycosaminoglycan per kilogram of patient, or is present in any range between any two of these amounts, in an amount lesser than or greater than any two of these amounts, or in an amount lesser than or equal to, or greater than or equal to any two of these amounts. In a preferred embodiment, the one or more glycosaminoglycans are present in the composition in an amount of about 0.25 to 4, 0.5 to 3.75, or 0.75 to 3.5 mg/kg of patient.

5. Other Components

The compositions of the present embodiments may contain additional components, including additional active components that act to enhance resveratrol biological activity and inactive compounds (e.g., flavorants, sweeteners, dyes, vitamins, amino acids (e.g., lysine, proline, etc.), minerals, nutrients, etc.). For example, tocopherols such as Vitamin E, sunflower lecithin, grape seed extract, cocoa extract, lutein, and green tea extract are preferred additional components in certain embodiments. Emulsifiers, fillers, binding agents, and the like may also be included in the compositions of the present embodiments.

The combination of the present embodiments is intended for human or animal oral intake as a dietary supplement. For example, such compositions may comprise a combination of resveratrol and hyaluronan in a dietary supplement that serves to heal a variety of illnesses including some cancers. Resveratrol is known to be an anti-cancer molecule and to have other healing and longevity enhancing properties. Hyaluronan (hyaluronic acid, HA) is taken as an oral supplement or can be given intravenously to target cancer cells. When combined with or attached to other molecules, hyaluronan will deliver other anti-cancer and healing agents such as resveratrol to tumor sites. The combination may or may not include a chelating agent, an antioxidant and/or an emulsifier. When encapsulated or otherwise applied together, with or without those additives, resveratrol and HA have powerful healing properties for animals and humans.

Most preferably, the compositions of the present embodiments stabilize resveratrol specific activity such that the resveratrol of the compositions has a specific activity that is greater than that of resveratrol maintained in the presence of oxygen gas, or maintained in the absence of a chelator, hyaluronic acid, or vitamin D. Preferably, the amounts of the non-resveratrol constituents of the compositions will stabilize the composition's resveratrol so that it exhibits at least 10% more activity, at least 20% more activity, at least 50% more activity, at least 2-times the activity, at least 5-times the activity, or at least 10-times the activity of resveratrol maintained in the presence of oxygen gas, or maintained in the absence of a chelator, hyaluronic acid, or vitamin D and so that it remains capable of exhibiting such specific activity over extended periods (for example, 1, 2, 4, 6, 10, 12, 18, 24, or 36 months or longer) at ambient conditions of temperature and humidity (i.e., without need for special precautions as to temperature or humidity).

In a preferred embodiment, the composition comprises or consists essentially of one or more plant extracts comprising trans-resveratrol and one or more of the following: a chelator such as phytic acid; one or more additional phenolic antioxidants such as quercetin or ferulic acid (ferulate); and Vitamin D. These compositions exhibit numerous benefits as compared to pure resveratrol alone. A particular benefit, explained in detail in Example 6 below, is that the present compositions do not exhibit the hormetic action characteristic of resveratrol (a dose-response relationship that is stimulatory at low doses, but detrimental at higher doses resulting in a J-shaped or an inverted U-shaped dose response curve). Instead, the present compositions have an L-shaped dose response curve, meaning that they are safe (non-toxic) even at high doses.

Preferred compositions comprise resveratrol (preferably, a composition dosage of from about 10 mg to about 2 g, more preferably from about 100 mg to about 500 mg), and at least one compound selected from the group consisting of an chelator, a glycosaminoglycan (e.g., hyaluronic acid), and vitamin D, and may also comprise other compounds such as antioxidants, emulsifiers, etc.

### B. Methods of Treatment

The administration of the compositions of the present invention may be for a “prophylactic” or “therapeutic” purpose. The compositions of the present invention are said to be administered for a “therapeutic” purpose if the amount administered is physiologically significant to provide a therapy for an actual manifestation of the disease. When provided therapeutically, the composition is preferably provided at (or shortly after) the identification of a symptom of actual disease. The therapeutic administration of the compound serves to attenuate the severity of such disease or to reverse its progress. The compositions of the present invention are said to be administered for a “prophylactic” purpose if the amount administered is physiologically significant to provide a therapy for a potential disease or condition, e.g., to reduce the risk of heart attacks, to maintain health, to sustain a youthful appearance, to sustain function (e.g., to sustain a certain level of visual acuity, etc. When provided prophylactically, the composition is preferably provided in advance of any symptom thereof. The prophylactic administration of the composition serves to prevent or attenuate any subsequent advance of the disease.

Providing a therapy or “treating” refers to any indicia of success in the treatment or amelioration of an injury, pathology or condition, including any objective or subjective parameter such as abatement, remission, diminishing of symptoms or making the injury, pathology or condition more tolerable to the patient, slowing in the rate of degeneration or decline, making the final point of degeneration less debilitating, or improving a patient's physical or mental well-being. The treatment or amelioration of symptoms can be based on objective or subjective parameters, including the results of a physical examination, neuropsychiatric examination, and/or laboratory methods.

Preferred subjects for treatment include animals, most preferably mammalian species such as humans, and domestic animals such as dogs, cats and the like, subject to disease and other pathological conditions. A “patient” refers to a subject, preferably mammalian (including human). In a preferred embodiment, the subject or patient is a human, and in a more preferred embodiment, the subject or patient is a human having or at risk of developing one or more of cardiovascular disease, cancer, macular degeneration, aging, neurodegenerative diseases (e.g., Alzheimer's Disease, Parkinson's Disease, etc.) and inflammation.

A variety of administration routes for the compositions of the present invention are available. The particular mode selected will depend, of course, upon the particular therapeutic agent selected, whether the administration is for prevention, diagnosis, or treatment of disease, the severity of the medical disorder being treated and dosage required for therapeutic efficacy. The methods of the present embodiments may be practiced using any mode of administration that is medically acceptable, and produces effective levels of the active compounds without causing clinically unacceptable adverse effects. Such modes of administration include, but are not limited to, oral, buccal, sublingual, inhalation, mucosal, rectal, intranasal, topical, ocular, periocular, intraocular, transdermal, subcutaneous, intra-arterial, intravenous, intramuscular, parenteral, or infusion methodologies. In a preferred embodiment, administration is oral.

The dosage schedule and amounts effective for therapeutic and prophylactic uses, i.e., the “dosing regimen”, will depend upon a variety of factors, including the stage of the disease or condition, the severity of the disease or condition, the general state of the patient's health, the patient's physical status, age and the like. In calculating the dosage regimen for a patient, the mode of administration also is taken into consideration. The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108). The state of the art allows the clinician to determine the dosage regimen for each individual patient, therapeutic agent and disease or condition treated. Single or multiple administrations of the compositions of the present invention can be administered depending on the dosage and frequency as required and tolerated by the patient. The duration of prophylactic and therapeutic treatment will vary depending on the particular disease or condition being treated. Some diseases lend themselves to acute treatment whereas others require long-term therapy.

The compositions of the present embodiments may be administered to a subject alone, or to a subject who is or will receive another medicament or medical therapy. For example, in a preferred embodiment, the compositions of the present embodiments are co-administered to a subject with stem cell therapy or a treatment for macular degeneration or macular dystrophy. Co-administration may be simultaneous, serially, contemporaneously, or in any other suitable fashion.

In a preferred embodiment, said administration or co-administration provides a therapeutic or prophylactic benefit to the subject that is at least 1.5 fold, 2 fold, 2.5 fold, 3 fold, 3.5 fold, 4 fold, 4.5 fold, 5 fold, 5.5 fold, 6 fold, 6.5 fold, 7 fold, or more than 7 fold greater than the therapeutic or prophylactic benefit achieved by resveratrol alone, calorie restriction alone, or the other medicament or medical therapy (e.g., stem cell therapy or treatment of macular degeneration or macular dystrophy) alone. In another preferred embodiment, said co-administration provides a therapeutic or prophylactic benefit to the subject that is at least 125 percent, 150 percent, 175 percent, 200 percent, 250 percent, 300 percent, 350 percent, 400 percent, 450 percent, 500 percent, or more than 500 percent greater than the therapeutic or prophylactic benefit achieved by resveratrol alone, calorie restriction alone, or the stem cell therapy or treatment of macular degeneration or macular dystrophy alone.

The compositions of these embodiments enhance resveratrol's specific activity. The compositions of the present embodiments therefore find utility in the treatment or prophylaxis of diseases (or in the amelioration of the symptoms of diseases) such as cardiovascular disease, cancer, macular degeneration, aging, neurodegenerative diseases (e.g., Alzheimer's Disease, Parkinson's Disease, etc.) and inflammation in which the modulation of expression of “survival/longevity” genes and/or “damage inducing” genes is desired. Over time, as minerals such as calcium and iron accumulate in the human body, genes respond in deleterious ways. Liu, Y. et al. (2005) Ann. Clin. Lab. Sci. 35 (3):230-239; Templeton, D. M. et al. (2003) Biochim. Biophys. Acta. 1619 (2):113-124; Ikeda, H. et al. (1992) Hepatology 15(2):282-287. The present embodiments have particular utility in the treatment of macular degeneration, cancer and the conditions of aging.

Additional embodiments provide a method of ameliorating a symptom associated with an existing disease of an individual or for preventing the onset of the symptom in an individual prior to the occurrence of the disease in the individual, which comprises administering to the individual, a resveratrol-containing composition that modulates the concentration or activity, relative to resveratrol alone or calorie restriction, of the product of a survival/longevity gene or the product of a gene whose expression enhances cellular damage, wherein the resveratrol is provided in an amount effective to cause a modulation of the concentration or activity of the gene that ameliorates the symptom of the disease, and wherein the disease is selected from the group consisting of: cardiovascular disease, cancer, macular degeneration, a disease associated with aging, and inflammation. The embodiments further provide such methods wherein the disease is cancer, or a disease associated with aging (especially a neurodegenerative disease).

1. Stem-Cell-Related Methods

In one preferred embodiment, the compositions of the present embodiments are co-administered to a subject with cell implantation or transplant therapy such as stem cell implantation or injection. The cells may be stem cells or cells derived from stem cells, such as human embryonic stem cells, or adult stem cells such as bone marrow stem cells, cardiac stem cells, endothelial stem cells, hematopoietic stem cells, mammary stem cells, mesenchymal stem cells, neural crest stem cells, neural stem cells, olfactory adult stem cells, testicular stem cells, and very small embryonic-like “VSEL” stem cells, or combinations thereof, or cells derived from any of the foregoing. In a preferred embodiment, the transplanted cells are selected from the group consisting of cardiac stem cells, neural stem cells, and retinal pigment epithelial (RPE) cells.

The therapeutic benefits that may be shown in such cell transplant-related embodiments include one or more benefits selected from the group consisting of improved stem cell differentiation, improved cell adhesion, improved cell survival, improved cell proliferation, and combinations thereof.

Stem cells are recognized as the origin of all renewed cells in the human body. Stem cell implantation is believed to be of benefit in regeneration of damaged tissues, particularly for brain or heart tissue damaged by infarction or trauma, or tissue that does not normally exhibit rapid cell renewal and turnover. Chacko et al., Am. J. Physiol. Heart Circ. Physiol. 2009 396 (5):H1263-73; Wakabayashi et al., J. Neurosci. Res. 2010 88 (5):1017-25.

It is known that stem cell implantation has exhibited only limited or modest benefit in regeneration of damaged tissues, such as following a heart attack, and that animals treated with injected stem cells often progress to heart failure within weeks of stem cell implantation. Assmus et al., New England J. Med. 2006 355 (12):1222-32; Shake et al., Ann. Thorac. Surg. 2002 73 (6):1919-25. However, there does appear to be a reduction in short-term mortality exhibited by injection of stem cells in the oxygen-deprived (ischemic) cardiac tissue. Assmus et al., Circ. Res. 2007 100 (8):1234-41.

It is also known that implanted stem cells must adhere to existing cell matrixes to facilitate tissue regeneration, and that free radicals impair stem cell tissue adherence. Song et al., Stem Cells 2010 28 (3):555-63. Further, free radicals inhibit stem cell differentiation into desired cells (e.g., heart muscle, brain neuron, etc), and antioxidants have been demonstrated to enhance stem cell differentiation. Id.

Antioxidants have been demonstrated to reduce free radicals and improve stem cell adhesion and stem cell survival during and following implantation. Song et al., Stem Cells 2010 28 (3):555-63; Rodriguez-Porcel et al., Mol. Imaging. Biol. 2010 12 (3):325-34; Kashiwa et al., Tissue Eng. Part A. 2010 16 (1):91-100. It is also known that resveratrol, a small molecule, enhances activity of endogenous antioxidants such as glutathione. superoxide dismutase (particularly manganese SOD), and catalase, and up-regulates the synthesis of stem cells themselves. Kao et al. Stem Cells Dev. 2010 19 (2):247-58.

It has been demonstrated in animals that orally administered resveratrol helps to maintain a reduced cellular environment (less free radical activity) at a relatively low dose concentration (2.5 mg per kilogram of body weight, 175 mg per 160-lb human) which results in improved stem cell survival and enhanced cardiac function (ejection fraction, etc.). Gurusamy et al., J. Cell. and Mol. Medicine. 14 (9):2235-39 (2010). In particular, Gurusamy et al. reported that pre-treatment of rats with low dose resveratrol for two weeks prior to injection of cardiac stem cells into the myocardium significantly improved cardiac functional parameters such as left ventricular ejection fraction and fractional shortening. Pre-treatment also enhanced stem cell survival and proliferation as demonstrated by differentiation of stem cells towards the regeneration of the myocardium.

In accordance with a preferred embodiment of the present invention, a matrix of small molecule antioxidants is combined with other small molecules and vitamin D3, and administered orally to preserve stem cells following implantation. Specifically, the matrix of small-molecule oral antioxidants includes, but is not limited to, resveratrol. This matrix is combined with other small molecules such as quercetin, IP6 phytate (inositol hexaphosphate), ferulic acid, EGCG (green tea), caffeic acid, apigenin, in combination with the vitamin/hormone vitamin D3. This combination exerts unexpected synergistic ability, over and above the expected additive properties of the individual constituents, to preserve stem cells following their implantation.

The dosage concentrations are lower than would be thought to be necessary from prior art experiments, thereby attesting to the synergism resulting from the combined constituents. For example, the dosage range of resveratrol in the combination is approximately 1.0 mg to approximately 5.0 mg/kilogram of body weight, and the total dosage concentration of all molecules is approximately 1.0 mg to approximately 5.0 mg/kilogram of body weight. The results of administering this mixture include greater genomic response, and improved tissue function (i.e., heart muscle activity—ejection fraction) equal to or greater than what has been exhibited in prior experiments. The mixture of constituents is preferably provided in a capsule but may be in pill, tablet, or liquid form.

2. Macular Degeneration

The prolongation of the human lifespan over the past few decades in the US has spawned the proliferation of macular degeneration, an age-related eye disease. While not resulting in total vision loss, the disease robs older adults of their central vision used for reading as well as color vision. Macular degeneration affects the visual center of the eye, called the macula. The macula is part of the retina where color-vision cells (cones) are located.

In a preferred embodiment, the compositions of the present embodiments are co-administered to a subject with one or more macular degeneration or macular dystrophy treatments selected from the group consisting of an anti-angiogenic medicament (e.g., anecortave acetate, bevacizumab, bevasiranib, pegaptanib sodium, ranibizumab, etc.), an anti-drusen medicament (e.g., ARC1905, copaxone, eculizumab, fenretinide, RN6G, etc.) implantation of a miniature telescope into the eye, laser photocoagulation, photodynamic therapy, or administration of another therapy such as alprostadil, AREDS2, cortical implants, macular translocation, micro-electrical stimulation, NT-501, photobiomodulation, radiation therapy, retinal implants or transplants, rheopheresis, cell transplantation (e.g., RPE cell transplantation, stem cell transplantation, etc.), submacular surgery, or a combination thereof.

The therapeutic benefits that may be shown in such macular-related embodiments include one or more benefits selected from the group consisting of preserved or improved eyesight (e.g., visual acuity), shrinkage or halting enlargement of visual defects, sparing cells in the central macula, permitting normal functioning of tissues surrounding or adjacent to the macula, decreases or prevention of increases in the amount of drusen or amyloid beta in the eyes, improving or increasing blood flow to the eye (and particularly the macula and retina), inhibition of blood vessel growth and leakage (e.g., angiogenesis), inhibition of scarring, improved retinal function, prevention or slowing of macular degeneration, prevention or slowing of cell death particularly retinal cells, reduction or elimination of eye lesions (e.g., geographic atrophy lesions), and combinations thereof.

Macular degeneration is a progressive, age-related disease that can be broken down into four stages. In the first stage, beginning in about the third decade of life, the inability of the “garbage cleaning” cells, called the retinal pigment epithelia (RPE), to engulf and remove cellular debris from the back of the eyes, results in the formation of small microscopic deposits called lipofuscin. Lipofuscin is formed by iron and copper-induced oxidation of cellular debris and its accumulation correlates with premature aging and shortened lifespan of organisms. The prevalence of macular degeneration is greater in Caucasians than persons with darkly-pigmented skin and Caucasians have more lipofuscin deposits in their retinas. Some of this cellular debris in the retina is comprised of used-up vitamin A that is shed from night-vision (rod) cells each morning in the human eye. The failure of the RPE cells to function results from accumulation of iron and calcium within the RPE. In the second stage, in about the fifth decade of life, there is progressive calcification of an underlying cellophane-thin retinal layer called Bruch's membrane, which resides between the RPE and the blood supply layer (choroid). While drusen that forms within the retina is partially composed of cholesterol, this lipid does not originate from the blood circulation or the liver where most cholesterol is produced. Calcifications within Bruch's membrane further impairs the exit of lipids (fats), protein, and cellular debris, from the photoreceptor layer, which results in the formation of yellow spots called drusen on the retina. Drusen can be observed during an eye examination using an ophthalmoscope. There is currently no method of removing drusen.

The death of the RPE cells is the third stage of this progressive disease. This is sometimes called RPE dropout. As the RPE cells are either impaired or have died, and Bruch's membrane is clogged with calcium, the photoreceptors then cannot be nourished and also begin to die off. There is currently no treatment for stages 1-3 of macular degeneration. Stage 1-3 is called the “dry” form of macular degeneration because it has not resulted in hemorrhage or edema or new blood vessel formation. About 85% of macular degeneration patients have the “dry” form of this disease. In the fourth stage, as breaks in Bruch's membrane occur, or Bruch's membrane becomes totally calcified, the photoreceptor layer is deprived of oxygen and new blood vessels form (called neovascularization) which can invade the photoreceptor layer in the macula and impair vision; or there may be leakage of blood serum or frank release of red blood cells, which results in edema or hemorrhage. This is the more advanced and sight-threatening form of macular degeneration, often called “wet” macular degeneration because of the presence of the leakage of blood serum or red blood cells into the photoreceptor layer. This stage of the disease, if caught early, can be treated with laser beams, which can seal up leaky blood vessels. However, this treatment is only effective in delaying the progression of the disease, not curing it.

The cell cleansing process facilitated by the lysosomes cannot keep up with the accumulation of metabolic waste over a lifetime. The parafoveal ring, where rod cell density is highest, and therefore more discs of used-up vitamin A are shed, is where macular degeneration begins, and where the highest concentration of lipofuscin is observed in the retina. Eventually, the RPE cells die off with advancing age, which increases the burden on the remaining RPE cells to maintain a healthy retina.

In the past, lipofuscin has been considered a harmless wear-and-tear byproduct of cellular metabolism. One aspect of the present embodiments relates to the recognition that lipofuscin, which forms from iron and copper-induced oxidation, and hardens within lysosomal bodies within retinal pigment epithelial cells, sensitizes the retina to damage by mild amounts of radiation and oxidation. The retina becomes increasingly sensitive to blue-light damage with advancing age. Drusen formation within the retina is associated with RPE cell inability to produce superoxide dismutase, an endogenous antioxidant enzyme. Mice deficient in superoxide dismutase develop features that are typical of age-related macular degeneration in humans. Superoxide dismutase protects retinal cells against unbound (free) iron. High iron diets and cellular environments have been shown to reduce superoxide dismutase activity.

Retinal photoreceptors and retinal pigment epithelial cells are believed to be especially vulnerable to damage by low-molecular weight complexes of iron. Since antioxidants in the blood circulation may not always be able to cross the blood-retinal barrier, the retina produces its own protective antioxidants that bind iron. Iron chelators inhibit the adverse effects of unbound (free) iron (not bound to proteins). Heme oxygenase also serves in a similar manner to iron chelators to prevent retinal damage induced by loose iron.

Numerous agents have been used experimentally to clear up lipofuscin and drusen. Statin drugs, commonly used to reduce blood serum levels of cholesterol, have also been tested to prevent lipofuscin deposits in animals. Statin drugs reduced lipofuscin formation but were toxic to the liver and brought about the early death of these animals. Piracetam, a derivative of the neurotransmitter GABA, now available as a dietary supplement, has been used successfully to reduce lipofuscin formation in brain tissues. Sorbinil is an enzyme inhibiting drug (aklose reductase inhibitor) that underwent unsuccessful human trials in the 1990s to prevent retinal problems associated with diabetes. Sorbinil has been shown to partially reduce lipofuscin deposits in the retinal pigment epithelium cells of rodents. Hydergine is a drug used to treat senile dementia. In a rodent study, hydergine was reported to have reduced brain lipofuscin levels, but also led to the early demise of the animals. The East Indian spice turmeric contains an antioxidant molecule called curcumin. Curcumin has been used in an experimental mouse study to reduce lipofuscin in the brain. Purslane is a flowering plant rich in magnesium, beta carotene and omega-3 oil. The provision of purslane to mice has been shown to reduce lipofuscin deposition in the brain of mice. In a lab dish study, sulforaphane, an antioxidant molecule found in Brussels sprouts and broccoli in 1992, has been used successfully to reduce lipofuscin deposits in RPE cells exposed to blue light.

Intraperitoneal administration of lipoic acid to aged rats leads to a reduction and elevation in lipofuscin and enzyme activity, respectively, in the cortex, cerebellum, striatum, hippocampus, and hypothalamus of the brain. These results suggest that lipoic acid, a natural metabolic antioxidant, should be useful as a therapeutic tool in preventing neuronal dysfunction in aged individuals. Lipoic acid, a natural antioxidant produced within living tissues, and also available as a dietary supplement, has been shown to protect RPE cells from oxidative damage in lab dish studies.

Lipofuscin formation dramatically increases in brain tissues following alcohol consumption. Supplementation with high-dose grape seed flavonols prevents increase lipofuscin formation. Lipofuscin is an end-product of lipid peroxidation which dramatically increases following ethanol consumption. Oolong and green tea drinks reverse the cognitive impairment and lipofuscin formation in mice. Epigallocatechin-3-gallate (EGCG), the major constituent of green tea, upregulates the activity of heme oxygenase in lab dish studies. Heme oxygenase is a protective enzyme against iron-induced oxidation, which occurs in the retina. It has been shown that the provision of supplemental estrogen decreases lipofuscin deposition in brain tissues. In a lab dish study, the provision of lutein and zeaxanthin to RPE cells reduced lipofuscin formation. In rodents given supplemental acetyl-L-carnitine, a decline in lipofuscin deposits has been measured in brain cells.

U.S. Pat. No. 5,747,536 describes the combined therapeutic use of L-carnitine, lower alkanoyl L-carnitines or the pharmacologically acceptable salts thereof, with resveratrol, resveratrol derivatives or resveratrol-containing natural products, for producing a medicament for the prophylaxis and treatment of cardiovascular disorders, peripheral vascular diseases and peripheral diabetic neuropathy. Melanin is an iron-binding antioxidant in the retina. As melanin levels decline in the retina with advancing age, there is a greater accumulation of lipofuscin.

In one embodiment, the present embodiments relates to a composition comprising a combination of: (a) a chelator such as inositol hexaphosphate (IP6), trans resveratrol, quercetin, or any polyphenol or bioflavonoid for metal(s) such as iron, copper, heavy metals; (b) a calcium chelator, such as inositol hexaphosphate (IP6); (c) a heme oxygenase activator, such as trans resveratrol, piceatannol, or any of resveratrol's natural analogs, or similar small molecules such as fisetin, myricetin, quercetin or other bioflavonoids; (d) an agent that lowers the affinity of oxygen for red blood cells, such as inositol hexaphosphate (IP6); and, optionally (e) other antioxidants such as vitamin E, lutein/zeaxanthin, alpha lipoic acid. The formulation functions to: (1) limit oxidation in retinal tissues (photoreceptors, retinal pigment epithelial cells (RPE), choroid, specifically mitochondria and lysosomes in RPE cells); (2) inhibit accumulation of lipofuscin deposits; (3) inhibit formation of drusen; and (4) limit calcifications to retinal tissues, especially Bruch's membrane.

3. Cancer

A major challenge in cancer therapy is to selectively target cytotoxic agents to tumor cells (Luo, Y. et al. (2000) Biomacromolecules 1 (2):208-218). To decrease undesirable side effects of small molecule anticancer agents, many targeting approaches have been examined. One of the most promising methods involves the combination or covalent attachment of the cytotoxin with a macromolecular carrier, and in particular with hyaluronic acid (Luo, Y. et al. (1999) Bioconjug. Chem. 10 (5):755-763; Luo, Y. et al. (1999) Bioconjug. Chem. 12 (6):1085-1088; Luo, Y. et al. (2002) Pharm. Res. 19 (4):396-402).

In one embodiment, the present embodiments relates to a resveratrol- and hyaluronic acid-containing composition for the treatment of cancer comprising: resveratrol, hyaluronan, and optionally vitamin D and/or IP6. It is believed that these components act synergistically with one another to mediate an effect in curing and/or in preventing cancer in humans and/or in improving immunity (e.g., immune system response) in patients threatened by tumors. This aspect of the present embodiments is based in part upon the recognition that natural molecules can boost cancer immunity, possibly in a manner similar to that observed in cancer-proof mice.

Upon provision with such composition, the sentinels of the innate immune system, dendritic cells, can be alerted and neutrophils, macrophages and natural killer cell activity can be significantly enhanced. The enhancement of vitamin D receptors via resveratrol is yet another major advantage of a combination approach to treat or prevent cancer. This approach appears to be more appropriate for senior adults, the highest risk group for cancer, who are often immune-compromised due to poor nutrition or lack of nutrient absorption. The fact that this therapy can now be immediately measured for effectiveness by non-invasive cancer cell counting technology means that expensive and equivocal tests on animals may not be required to prove efficacy.

Vitamin D exhibits many biological actions. While vitamin D is widely known for its ability to stave off bone disease (rickets in growing children, osteoporosis in senior adults), it is becoming a central player in the battle against cancer. Only recently is it also gaining attention as an antibiotic. Vitamin D-deficient mice exhibit a defective response from phagocyte cells in the face of infection or inflammation. Vitamin D deficiency is frequently associated with recurrent infections. Only about half of the macrophage cells accumulate at the site of inflammation in vitamin D-deficient animals compared to animals whose vitamin D levels are adequate.

To delve deeper into the role of vitamin D in immunity and cancer, vitamin D improves the chemotactic (affinity for) neutrophils to mobilize and migrate. Patients with rickets due to vitamin D deficiency are observed to have sluggish neutrophils that cannot migrate properly. Vitamin D stimulates the maturation of monocytes to macrophages. This results in an enlarged army of immune fighting cells to mount against tumors. Greater attention is now being given to vitamin D as an anti-cancer weapon because of studies which show supplemental vitamin D drastically reduces the risk for all types of cancer. A study that employed 1100 IU of vitamin D3 produced a 60-77% reduction in cancer risk among women in Nebraska in just a 4-year period.

Even though cancer risk is lowest in sunnier and Equatorial areas geographically, where vitamin D levels are higher in sun-exposed populations, the protective effect of vitamin D against cancer has been repeatedly dismissed or discounted. The consumption of vitamin D orally eliminates the concern of skin cancer emanating from overexposure to unfiltered sun rays. One of the latest analyses shows that the risk of colon cancer can be halved by taking 2000 IU of vitamin D per day and that the risk for breast cancer can be halved by taking 3500 IU of vitamin D per day. The median dietary intake of vitamin D is only about 230 IU per day, so the prospect of food fortification or supplementation to prevent or treat cancer now becomes real.

In order for tissues to utilize and benefit from vitamin D they must have proteins in their outer coat (cell membrane) that are designed to receive and bind to vitamin D. For example, about 80% of human breast tumors produce vitamin D cell receptors, though gene expression (production) of vitamin D receptor is at low levels. Vitamin D's ability to inhibit cancer may be heightened when it is aided by weak estrogen-like molecules in the diet. Resveratrol, an estrogen-like molecule commonly found in red wine, upregulates the vitamin D receptor in breast cancer cells without increasing cancer growth. Resveratrol, in effect, can sensitize breast cancer cells to the anti-cancer properties of vitamin D.

Laboratory experiments show that low-dose vitamin D3 does not reduce breast tumor cell growth but when combined with resveratrol, tumor cell numbers declines by 40%. At higher concentrations vitamin D3 reduces the number of breast cancer cells in a lab dish by about 25%, and this decline improves to 50% when combined with resveratrol. Whereas estrogen increases vitamin D receptor gene expression, it also stimulates breast tumor growth. Resveratrol does not have this drawback. Resveratrol potentiates or “weaponizes” the cancer-inhibiting effect of vitamin D. Furthermore, resveratrol by itself has been shown to calm the response of phagocytes to foreign invaders like germs and tumor cells. Resveratrol dampens production of reactive oxygen species (free radicals) and normalizes particle ingestion in macrophage cells. Therefore, resveratrol prevents the over-response of immune cells that can produce autoimmunity.

Resveratrol blocks cancer in so many ways that it is difficult to find a pathway for cancer that is not obstructed by resveratrol. Resveratrol induces the cell energy compartments in tumor cells, called mitochondria, to release an enzyme called cytochrome C oxidase that usually leads to a cascade of other enzymes that induce programmed cell death, called apoptosis. But a recent experiment also shows that resveratrol releases cytochrome C from ovarian tumor cells that leads to rapid cell death via a process called autophagy, a process where enzymes produced inside the tumor cell actually digest its innards (kind of a form of intracellular cannibalism). This is a form of cell suicide that resveratrol activates in tumor cells, but not healthy cells.

The contribution of innate immunity in surveillance of tumors is comparatively neglected in cancer biology. Phagocytosis, or “cell eating” is the cornerstone of the innate immune response. Focus has been directed to dendritic cells which are believed to be sentinels of the innate immune response. A limited number of immune-boosting agents have been investigated.

Skepticism surrounds interest in innate immune approaches to cancer treatment. For example, patients taking immune-suppressing don't necessarily develop cancer with more frequency. However, this may be misunderstood. An over-responsive immune system may lead to more tissue and organ damage that can be mortal to cancer patients. Most of the drugs used for breast cancer therapy induce immune suppression.

Nature's most potent iron chelator is inositol hexaphosphate (IP6), which is found in seeds and the bran fraction of whole grains. A low dosage of IP6 has been found to suppress the growth of rhabdomyosarcoma cells by 50%. Removal of IP6 allows these tumor cells to recover and grow once again. IP6-treated mice with injected tumors exhibit tumors that are 50 times smaller than non-treated mice. IP6 has also been shown to reduce the growth of injected fibrosarcoma cells in mice and prolong their survival. In examining the immune enhancing properties of IP6 it has been shown that it boosts production of free radicals (superoxide) and the cell digesting action of neutrophils in the presence of bacteria. IP6 increases the release of interleukin-8. The action of natural killer cells, which are involved in tumor cell destruction, is enhanced by IP6.

In one embodiment, the hyaluronic acid of such composition is conjugated to a chemotherapeutic agent. The embodiments particularly pertain to such compositions in which the chemotherapeutic agent is taxol. The embodiments particularly pertain to such compositions that additionally and preferably comprise a chelator, and/or vitamin D. Most malignant solid tumors contain elevated levels of Hyaluronic Acid (Rooney, P. et al. (1995) Int. J. Cancer 60 (5):632-636) and these high levels of HA production provide a matrix that facilitates invasion (Hua, Q. et al. (1993) J. Cell. Sci. 106 (Pt 1):365-375; Luo, Y. et al. (2000) Biomacromolecules 1 (2):208-218). Thus chemotherapeutic agents that are conjugated to Hyaluronic Acid target tumor cells, and can provide an effective anti-tumor dosage at lower overall concentration.

In brief, a preferred method of conjugation entails forming an NHS (N-hydroxy-succimimide derivative of the chemotherapeutic agent. Such a derivative can be made by adding a molar excess of dry pyridine to a stirred solution of Taxol and succinic anhydride in CH₂Cl₂ at room temperature. The reaction mixture is then stirred for several days at room temperature and then concentrated in vacuo. The residue is dissolved in 5 ml of CH₂Cl₂ and the produced Taxol-2′-hemisuccinate can be purified on silica gel (washed with hexane; eluted with ethyl acetate) to give the desired product (Luo, Y. et al. (1999) Bioconjug. Chem. 10 (5):755-763).

The N-hydroxy-succimimide derivative of the chemotherapeutic agent is then conjugated to adipic dihydrazido-functionalized hyaluronic acid. Adipic dihydrazido-functionalized hyaluronic acid is preferably prepared as described by Pouyani, T. et al. (1994) (Bioconjugate Chem. 5:339-347); Pouyani, T. et al. (1994) (J. Am. Chem. Soc. 116:7515-7522); Vercruysse, K. P. et al. (1997) (Bioconjugate Chem. 8:686-694). Thus, hyaluronic acid is preferably dissolved in water and an excess of adipic dihydrazide (ADH). The pH of the reaction mixture is adjusted to 4.75 by addition acid. Next, 1 equivalent of 1-Ethyl-3-[3-(dimethylamino)-propyl]carbodiimide (EDCI) is added in solid form. The pH of the reaction mixture is maintained at 4.75 by addition of acid. The reaction is quenched by addition of 0.1 N NaOH to adjust the pH of reaction mixture to 7.0. The reaction mixture is then transferred to pretreated dialysis tubing (Mw cutoff 3,500) and dialyzed exhaustively against 100 mM NaCl, then 25% EtOH/H2O and finally water. The solution is then filtered through 0.2 m cellulose acetate membrane, flash frozen, and lyophilized (Luo, Y. et al. (1999) Bioconjug. Chem. 10 (5):755-763).

4. Aging

Calcification and rusting of cells impairs the cleansing of cellular debris (lipofuscin) from cells by enzymes produced by lysosomes, and results in impairment of cellular energy (ATP) produced by the mitochondria within cells. The compositions of the present embodiments inhibit and/or reverse cellular aging and/or connective tissue aging, and in particular, inhibit and/or reverse cellular aging and/or connective tissue aging caused by an accumulation of major minerals (e.g., iron, calcium, etc.). As a consequence, recipients of the compositions of the present embodiments exhibit enhanced longevity and enhanced cellular and connective tissue health and structure.

The human body ages at the cellular level by the slow accumulation of cellular debris called lipofuscin, which is facilitated by the progressive accumulation of iron and calcium within lysosomes and mitochondria. A cell cleansing and renewal process called autophagy prevents the accumulation of lipofuscin during the years of youthful growth, but this lysosomal mechanism declines once full growth is achieved due to accumulation of intracellular iron and calcium. Progressive inability to remove cellular debris results declining cell function and then premature death of the cell. A young cell efficiently removes debris from within. An old cell cannot efficiently remove debris and accumulates lipofuscin. The mitochondria, which provides cellular energy for lysosomal bodies to perform their cell cleansing activity, also becomes progressively calcified and ironized once childhood growth ceases. Only about 5% of mitochondria are functioning by age 80. Iron and calcium chelators are proposed to remedy mitochondrial aging which impacts cellular functions such as lysosomal enzymatic activity

The human body ages within connective tissue by failure of cells called fibroblasts to regenerate collagen and hyaluronic acid, the latter being a space-filling, water-holding molecule. Collagen formation is facilitated by vitamins and amino acids in the diet (vitamin C, lysine, proline). Fibroblasts can be stimulated to produce hyaluronic acid by estrogen, made naturally in the body, and by estrogen-like molecules found in plants, called phytoestrogens, provided in the diet of by hyaluronic acid itself. Young females, by virtue of the ability to produce estrogen, exhibit thicker hair, smoother skin and more flexible joints, due to the abundance of hyaluronic acid. All of these being attributes of youthfulness.

The inability to regenerate hyaluronic acid results in tissues losing their physical integrity by virtue of loss of the space-filling properties of hyaluronic acid. Without adequate hyaluronic acid, a dehydrated state results and tissues shrink and shrivel up. For example, skin that is lacking hyaluronic acid will appear wrinkled and dry. Joint spaces will lack the cushioning and space-filling needed to prevent bone from rubbing on bone. The eyes will begin to shrink in size. Hair will thin due to the lack of hydration. These are the most prominent visible or cosmetic signs of aging.

In one embodiment, the present embodiments address both cellular and extracellular (connective tissue) aging, thus (a) preserving youthful function of living cells by removal of excess minerals, largely calcium and iron, from cells, this facilitating autophagy (cleanup of cellular debris, such as lipofuscin, via lysosomal enzymes) and (b) invigorating and preserving production of hyaluronan by stimulation of fibroblasts by HA, phytoestrogens (resveratrol, quercetin, genistein, are a few), to inhibition of degradation of HA by provision of metal chelators, such as phytic acid, ferulate, quercetin, resveratrol, etc.

In one embodiment, the dietary supplement addresses both cellular and extra-cellular aging by its ability to stimulate renewal of living cells from within via enzymatic degradation of cellular debris by intracellular lysosomal bodies. This is facilitated by the inclusion of metal (iron, copper, heavy metal) and calcium chelating molecules within the formula. Lysosomes lose their ability to enzymatically digest cellular debris with the progressive accumulation of iron, copper and other metals, and the crystallization of calcium. In another embodiment, the dietary supplement stimulates fibroblasts to produce hyaluronic acid at youthful levels again. This is accomplished by provision of orally-consumed molecules that stimulate fibroblasts to produce hyaluronic acid. In another embodiment, the dietary supplement includes metal chelating molecules that help maintain youthful lysosomal function are identified as antioxidants, like vitamin E or vitamin C, lipoic acid, metal chelators like IP6 phytate, quercetin, bioflavonoids or polyphenols, resveratrol. Resveratrol works by its ability to stimulate production of heme oxygenase, an enzyme that helps to control iron. The dietary supplement may also include molecules that inhibit crystallization of calcium are magnesium and IP6 phytate, and orally consumed molecules that stimulate fibroblasts to produce hyaluronic acid are hyaluronic acid, glucosamine, chondroitin, or estrogen-like molecules such as genistein, lignans, hydroxytyrosol, or other molecules configured like estrogen. Orally consumed HA stimulates greater HA and chondroitin synthesis. Similarly, glucosamine stimulate fibroblasts to produce HA. Alternatively, or additionally, glucosamine stimulates synovial production of hyaluronic acid, which is primarily responsible for the lubricating and shock-absorbing properties of synovial fluid” (McCarty, M. F. (1998) Medical Hypotheses 50:507-510, 1998). In yet another embodiment, the dietary supplement may include orally consumed molecules that stimulate production of collagen are vitamin C, proline and lysine.

In such embodiment, the present embodiments relate to a resveratrol and hyaluronic acid-containing dietary supplement that restores youthful function and appearance to human cells and tissue. The embodiments particularly pertain to such compositions that additionally comprise a chelator, and/or vitamin D. Most preferably, the composition will comprise the chelator phytic acid (inositol hexaphosphate; IP6). The compositions of the present embodiments synergistically enhance the specific activity of the resveratrol and/or hyaluronic acid, and thus the compositions of the present embodiments provide an enhancement of activity above and beyond that obtained with the components administered individually. In such embodiment, the embodiments relates to a method for restoring youthful function and appearance to human cells and tissues comprising the following steps: (a) stimulating renewal of living cells from within via enzymatic degradation of cellular debris by intracellular lysosomal bodies (preferably by providing a metal chelating molecule that helps maintain youthful lysosomal function, such molecules comprising antioxidants, such as vitamin E or vitamin C, lipoic acid, metal chelators like IP6 phytate, quercetin, bioflavonoids or polyphenols, and/or resveratrol); and (b) stimulating fibroblasts to produce hyaluronic acid (comprises providing orally consumed molecules that stimulate fibroblasts to produce hyaluronic acid, such orally consumed molecules comprising, for example, hyaluronic acid, glucosamine, chondroitin, and/or estrogen-like molecules such as genistein, lignans, hydroxytyrosol, or other molecules configured like estrogen). Preferably, such stimulation is achieved by the dietary administration of a composition comprising the stated compounds, more preferably in combination with an orally consumable molecule that stimulates production of collagen, such molecules comprising, for example, vitamin C, proline and/or lysine.

The individual components of the composition are believed to act synergistically to enhance the effect of, for example, resveratrol. Without intending to be limited thereby, it is proposed that the body's control or chelation of iron and calcium regulates the rate of aging after full growth has been achieved. During childhood growth all the iron and calcium are directed towards production of new bone and new red blood cells (hemoglobin). The cessation of childhood growth results in excess iron, copper and calcium, which then progressively (a) calcifies and (b) rusts tissues. The lysosomes begin to accumulate iron and calcium, which results in their dysfunction. The mitochondria begin to malfunction as they also progressively rust and calcify. The compositions of the present embodiments are believed to be capable of limiting or slowing the progressive rusting and calcification of cells and cellular organelles to thereby facilitate a slowing or reversal of the aging process. The chelation is what controls the genes. Genes are then favorably upregulated or downregulated. Resveratrol and a copper chelator are believed to act: (1) as controllers of calcium concentration via upregulation of osteocalcin, the hormone that helps retain calcium in bones and (2) as controllers of iron concentration via heme oxygenase, an antioxidant enzyme.

MAO inhibitors and iron chelators have been proposed as treatments for Parkinson's disease (Youdim, M. B. et al. (2004) J. Neural. Transm. 111 (10-11):1455-1471; Yáñez, M. et al. (2006) Eur. J. Pharmacol. 542 (1-3):54-60; Bureau, G. et al. (2008) J. Neurosci. Res. 86 (2):403-410; Singh, A. et al. (2003) Pharmacol. 68 (2):81-88; Gao, X. et al. (2007) Am. J. Clin. Nutr. 86 (5):1486-1494; Johnson, S. (2001) Med. Hypotheses 56 (2):171-173). The compositions of the present embodiments which contain the MAO inhibitor and copper chelator, resveratrol, the iron chelator and MAO inhibitor, quercetin, and the broad metal chelator, phytic acid are particularly preferred for the treatment of neurodegenerative diseases (especially Parkinson's Disease, camptocormia, and Alzheimer's Disease) or in the amelioration of the symptoms of such diseases.

### C. Modulation of Gene Product Concentration or Activity

In an example embodiment, the compositions are capable of modulating gene expression to an extent greater than that observed with resveratrol alone or with calorie restriction. In a preferred embodiment, the specific activity of the resveratrol in a resveratrol-containing composition has been stabilized or enhanced. As used herein, the term “specific activity” refers to the ratio of the extent of gene modulation (relative to control) per amount (mass) of administered resveratrol. In another preferred embodiment, the compositions up-regulate a survival/longevity gene or down-regulate a gene whose expression enhances cellular damage upon administration to a recipient.

The embodiments pertains to compositions that, upon administration to a recipient, increase the concentration or activity of a survival/longevity gene product and/or decrease the concentration or activity of a gene product that induces or causes cellular damage. As used herein, such increase (or decrease) in concentration or activity may be accomplished by any mechanism. For example, such increase (or decrease) may reflect a modulation of gene expression resulting in either increased (or decreased) expression of the gene encoding the survival/longevity gene product, or a gene that regulates (e.g., induces or represses) or whose product regulates such expression or activity. Alternatively, or conjunctively, such increase (or decrease) in concentration or activity may reflect a modulation of the recipient's ability to degrade or stabilize any such gene products. Alternatively, or conjunctively, such increase (or decrease) in concentration or activity may reflect a modulation of the recipient's ability to enhance, accelerate, repress or decelerate the activity of any such gene products.

The modulation of concentration or activity discussed above may be a modulation of intracellular, intercellular and/or tissue concentration or activity of such survival/longevity gene products or such gene products that induce or cause cellular damage. Such modulation may be identified by assays of DNA expression, assays of gene product activity, assays of the level of gene product, assays of the rate of gene product turnover, etc. conducted in one or more types of cells, tissues, etc.

An increase in the concentration of a survival/longevity gene product may result from, for example, increased transcription of the gene that encodes the survival/longevity gene product, increased transcription of a gene that induces the expression of the gene that encodes the survival/longevity gene product, decreased transcription of a gene that represses the expression of the gene that encodes the survival/longevity gene product, decreased degradation or enhanced stabilization of expressed molecules of the survival/longevity gene product (leading to the enhanced accumulation of the survival/longevity gene product). Similarly, a decrease in the concentration of a survival/longevity gene product may result from, for example, decreased transcription of the gene that encodes the survival/longevity gene product, decreased transcription of a gene that induces the expression of the gene that encodes the survival/longevity gene product, increased transcription of a gene that represses the expression of the gene that encodes the survival/longevity gene product, increased degradation or decreased stabilization of expressed molecules of the survival/longevity gene product (leading to the enhanced dissipation of the survival/longevity gene product).

One aspect of the present embodiments thus relates to the use of resveratrol and resveratrol-containing compositions to modulate gene expression, and in particular, to modulate the expression of “survival/longevity” genes and/or “damage inducing” genes. As used herein, a compound is said to “modulate” gene expression if its administration results in a change in expression (relative to a control) of such genes of at least 10%. Modulation may involve an increase in expression (“up-regulation”) or it may involve a decrease in expression (“down-regulation”). The term up-regulate thus denotes an increase of expression of at least 10%, at least 20%, at least 50%, at least 2-fold, at least 5-fold, or most preferably at least 10-fold (relative to a control). The term down-regulate conversely denotes a decrease of expression of at least 10%, at least 20%, at least 50%, at least 2-fold, at least 5-fold, or most preferably at least 10-fold (relative to a control).

A second aspect of the present embodiments relates to the use of resveratrol and resveratrol-containing compositions to modulate the concentration or activity of expressed products of “survival/longevity” genes and/or “damage inducing” genes. As used herein, a compound is said to “modulate” the concentration or activity of such expressed products if its administration results in a change in an intracellular, intercellular or tissue concentration or activity (relative to a control) of such gene products of at least 10%. Modulation may, for example, involve an “enhanced accumulation” or an “enhanced activity” or, for example, it may involve a “diminished accumulation” or a “diminished activity.” The term “enhanced accumulation” (or “enhanced activity”) denotes an increase in concentration (or activity) of at least 10%, at least 20%, at least 50%, at least 2-fold, at least 5-fold, or most preferably at least 10-fold (relative to a control). The term “diminished accumulation” or “diminished activity.” conversely denotes a decrease in concentration (or activity) of at least 10%, at least 20%, at least 50%, at least 2-fold, at least 5-fold, or most preferably at least 10-fold (relative to a control).

As used herein, a “survival/longevity” gene is a gene whose expression contributes to an increase in the survival or longevity of a subject (e.g., a mammal, and particularly a human) expressing such gene. Conversely, a “damage inducing” gene is a gene whose expression contributes to DNA, cellular, or tissue damage in such subject. Such genes are responders to biological stressors, they initiate action in response to stressors such as radiation (e.g., sunlight, gamma rays, UV light, etc.), radiomimetic agents (e.g., vitamin D), heat, near starvation (calorie restriction, or its mimetic, resveratrol) by modulating their expression.

In a preferred embodiment, the survival/longevity gene is a sirtuin gene. The sirtuins are a conserved family of deacetylases and mono-ADP-ribosyltransferases, which have emerged as key regulators of cell survival and organismal longevity. Mammals have at least seven sirtuins, including Sirtuins 1 through 7. Sirtuin 1 is a nuclear deacetylase that regulates functions including glucose homeostasis, fat metabolism and cell survival. The Sirtuin 1 gene is known to control the rate of aging of living organisms by virtue of its ability to produce DNA repair enzymes and mimics the beneficial effects of calorie restriction. The trans form of resveratrol (but not cis-resveratrol) activates the Sirtuin 1 gene. The Sirtuin 3 gene is a mitochondrial sirtuin that regulates acetyl-CoA synthetase 2, and thus its modulation has physiological applications including increasing mitochondrial biogenesis or metabolism, increasing fatty acid oxidation, and decreasing reactive oxygen species. The role of Sirtuin 3 in promoting cell survival during genotoxic stress was demonstrated in U.S. Patent Application Publication No. 2011/0082189. Preferred embodiments particularly pertain to compositions that modulate (increase or decrease) the concentration of the Sirtuin 1 or Sirtuin 3 survival/longevity gene products, particularly as compared to the ability of resveratrol alone to modulate the gene products.

In particular, commercial formulations (sold as Longevinex®) of the present embodiments have been shown to upregulate Sirtuin 3 at rates up to 2.95 times greater than resveratrol alone. Mukherjee et al., Can. J. Pharmol. Physiol. 2010 November; 88 (11):1017-25. Sirtuin3 protein regulates manganese superoxide dismutase (Mn SOD) within the mitochondria, which may have direct affect upon aging, function and survival of the mitochondria with advancing age and in states of disease. Data also suggests that the commercial Longevinex® formulations lowered C-reactive protein (marker of inflammation), reduced insulin, raised HDL cholesterol and abolished impairment of flow-mediated arterial dilatation, the first sign of atherosclerotic disease.

Examples of survival/longevity genes and genes whose expression enhances cellular damage include, e.g., the genes disclosed in Tables 1 and 2, respectively. Most preferably, such genes are human genes.

TABLE 1
Exemplary Survival/Longevity Genes

39329, 39340, 0610007C21Rik, 0610007L01Rik, 0610010F05Rik, 0610037L13Rik, 0610037P05Rik, 0610040B10Rik,
0610042E11Rik, 1110001A07Rik, 1110002B05Rik, 1110003O08Rik, 1110005A03Rik, 1110007L15Rik, 1110007M04Rik,
1110008F13Rik, 1110008J03Rik, 1110008P14Rik, 1110014K08Rik, 1110018J18Rik, 1110019J04Rik, 1110020G09Rik,
1110028A07Rik, 1110028C15Rik, 1110032E23Rik, 1110033M05Rik, 1110036003Rik, 1110038B12Rik, 1110038D17Rik,
1110054005Rik, 1110058L19Rik, 1110059E24Rik, 1110059G10Rik, 1110067D22Rik, 1190017O12Rik, 1300010M03Rik,
1300012G16Rik, 1500002101Rik, 1500002O20Rik, 1500005K14Rik, 1500011B03Rik, 1500011K16Rik, 1500031L02Rik,
1500034J01 Rik, 1600012F09Rik, 1600015H20Rik, 1600027N09Rik, 1700001O22Rik, 1700011B04Rik, 1700017H01Rik,
1700020C11 Rik, 1700021C14Rik, 1700021F05Rik, 1700023D09Rik, 1700029F09Rik, 1700029M20Rik, 1700030K09Rik,
1700040L02Rik, 1700051A21Rik, 1700113I22Rik, 1700127D06Rik, 1810007M14Rik, 1810011O10Rik, 1810012P15Rik,
1810013L24Rik, 1810015C04Rik, 1810020D17Rik, 1810021J13Rik, 1810022K09Rik, 1810026B05Rik, 1810029B16Rik,
1810030N24Rik, 1810034K20Rik, 1810035L17Rik, 1810044A24Rik, 1810049H13Rik, 1810058I24Rik, 1810059G22Rik,
1810063B05Rik, 1810073N04Rik, 2010106G01 Rik, 2010109N14Rik, 2010111I01Rik, 2010200O16Rik, 2010305A19Rik,
2010309E21Rik, 2010315B03Rik, 2010320M18Rik, 2010321M09Rik, 2210010L05Rik, 2210020M01Rik, 2210408I21Rik,
2310001A20Rik, 2310002L09Rik, 2310007O11Rik, 2310011J03Rik, 2310014D11Rik, 2310014F07Rik, 2310016C16Rik,
2310026E23Rik, 2310030G06Rik, 2310033F14Rik, 2310036O22Rik, 2310038H17Rik, 2310042E22Rik, 2310043N10Rik,
2310044H10Rik, 2310046A06Rik, 2310047A01Rik, 2310047H23Rik, 2310047M10Rik, 2310061J03Rik, 2310067B10Rik,
2310076G13Rik, 2410001C21Rik, 2410002O22Rik, 2410003K15Rik, 2410004B18Rik, 2410005O16Rik, 2410012H22Rik,
2410017P07Rik, 2410017P09Rik, 2410018C17Rik, 2410018C20Rik, 2410019A14Rik, 2410022L05Rik, 2410042D21Rik,
2510003E04Rik, 2510042H12Rik, 2610001J05Rik, 2610008E11Rik, 2610019F03Rik, 2610024B07Rik, 2610028D06Rik,
2610029101Rik, 2610030H06Rik, 2610101N10Rik, 2610200G18Rik, 2610209M04Rik, 2610301F02Rik, 2610507B11Rik,
2610528E23Rik, 2700029M09Rik, 2700038NO3Rik, 2700097O09Rik, 2810004N23Rik, 2810008M24Rik, 2810410M20Rik,
2810422O20Rik, 2810423A18Rik, 2810430I11Rik, 2810455D13Rik, 2900002H16Rik, 2900006B11Rik, 2900008C10Rik,
2900011GO8Rik, 2900024O10Rik, 3010003L21Rik, 3010027C24Rik, 3110003A17Rik, 3110031B13Rik, 3110043O21Rik,
3110073H01Rik, 3110080E11Rik, 3110082I17Rik, 3222402P14Rik, 3321401G04Rik, 4432414F05Rik, 4631424J17Rik,
4632404M16Rik, 4632411B12Rik, 4732416N19Rik, 4732418C07Rik, 4832420A03Rik, 4833408C14Rik, 4833439L19Rik,
4921506J03Rik, 4921509O07Rik, 4921513H07Rik, 4921517N04Rik, 4930402E16Rik, 4930426L09Rik, 4930429B21Rik,
4930432L08Rik, 4930432O21Rik, 4930448F12Rik, 4930453O09Rik, 4930455C21Rik, 4930466F19Rik, 4930486A15Rik,
4930505O20Rik, 4930513N20Rik, 4930523C07Rik, 4930524O07Rik, 4930544L04Rik, 4930551A22Rik, 4930554H23Rik,
4930557J02Rik, 4930570C03Rik, 4930570E01Rik, 4930573O21Rik, 4930579G24Rik, 4932442K08Rik, 4933402C05Rik,
4933403F05Rik, 4933404K13Rik, 4933407I18Rik, 4933411K2ORik, 4933413C19Rik, 4933421A08Rik, 4933426M11Rik,
4933428L01Rik, 4933429D07Rik, 4933433P14Rik, 4933434E20Rik, 4933440H19Rik, 5033414K04Rik, 5033421C21Rik,
5033423K11Rik, 5033430J17Rik, 5330423I11Rik, 5330439A09Rik, 5430402E10Rik, 5430402P08Rik, 5430407P10Rik,
5730470L24Rik, 5730507A11Rik, 5730536A07Rik, 5730601F06Rik, 5830404H04Rik, 5830415L20Rik, 5830428H23Rik,
5830432E09Rik, 5830436I19Rik, 5830457O10Rik, 5830469G19Rik, 5830487K18Rik, 5930434B04Rik, 6230429P13Rik,
6330403M23Rik, 6330407G11Rik, 6330409N04Rik, 6330415G19Rik, 6330417G04Rik, 6330503CO3Rik, 6330564D18Rik,
6330569M22Rik, 6430548M08Rik, 6530404N21Rik, 6530413G14Rik, 6620401M08Rik, 6720462K09Rik, 6720475J19Rik,
6820401H01Rik, 7030402D04Rik, 7030407E18Rik, 7420416P09Rik, 8030463A06Rik, 8030475D13Rik, 8430436O14Rik,
9030411M15Rik, 9030418K01Rik, 9030425P06Rik, 9130011J15Rik, 9230110F11Rik, 9230114K14Rik, 9330109K16Rik,
9330120H11Rik, 9430010O03Rik, 9430013L17Rik, 9530018H14Rik, 9530018I07Rik, 9530097N15Rik, 9930024M15Rik,
A030007L17Rik, A230046K03Rik, A230051G13Rik, A230062G08Rik, A230067G21Rik, A230091C14Rik, A330043J11Rik,
A330076H08Rik, A430005L14Rik, A430102J17Rik, A430110N23Rik, A530082C11Rik, A730008L03Rik, A830018L16Rik,
A930001N09Rik, A930006D11Rik, A930018M24Rik, A930026I22Rik, Ahcyl1, Amd1, Ank, Arhgap18, Arhgap20,
Arhgap24, Arhgap29, Arhgap4, Arhgap5, Arhgap9, Arhgdia, Arhgef1, Arhgef12, Arhgef17, Arhgef2,
B230117O15Rik, B230118H07Rik, B230219D22Rik, B230312A22Rik, B230337E12Rik, B230380D07Rik, B3galnt2,
B630005N14Rik, B830007D08Rik, B830028B13Rik, B930093H17Rik, C030002C11Rik, C030007101Rik, C030044B11Rik,
C030046101Rik, C130057M05Rik, C130065N10Rik, C230091D08Rik, C430003N24Rik, C730025P13Rik, Cdc73, Col10al,
Col19a1, Col1a1, Col1a2, Co123a1 , Col27a1 , Col4a3bp, Col5a1 , Col6a2, D030011O10Rik, D030051N19Rik,
D230019N24Rik, D330001F17Rik, D330017J20Rik, D430015B01Rik, D430018E03Rik, D530037H12Rik, D630023B12Rik,
D830046C22Rik, D930017J03Rik, D930020B18Rik, E030018N11Rik, E130014J05Rik, E130303B06Rik, E330021D16Rik,
E430010N07Rik, E430018J23Rik, EG226654, EG622645, EG633640, ENSMUSG00000050599, ENSMUSG00000071543,
ENSMUSG00000074466, ENSMUSG00000074670, ENSMUSG00000075401, Exdl1, G3bp1, Galnt3, Galnt4, Galntl4, Gart,
Kcna5, Kcna7, Kcng2, Kcnj3, Kcnj5, Kcnk3, Kcnv2, Kctd10, Kctd2, Kctd7, LOC100044376, LOC100044968,
LOC100045002, LOC100045020, LOC100045522, LOC100045629, LOC100046086, LOC100046343, LOC100046855,
LOC100047028, LOC100047385, LOC100047539, LOC100047601, LOC100047794, LOC100047915, LOC100048376,
LOC100048397, LOC100048439, LOC100048863, LOC640441, LOC668206, LOC675709, LOC677447, Mtm1,
OTTMUSG00000001305, OTTMUSG00000016644, P2ry5, P2ry6, Pabpc3, Pah, Paics, Paip1, Paip2, Palm, Papd1, Papd5,
Papola, Papolg, Paqr7, Paqr9, Pard3, Pard6g, Parp12, Pbef1, Pbld, Pbrm1, Pcbp2, Pcca, Pcdh7, Pcdh9, Pcgf3,
Pcgf6, Pcm1, Pcmt1, Pcnt, Pcnx, Pcp4, Pcp4I1, Pcsk7, Pctk1, Pctk2, Pctk3, Pdcd10, Pdcd4, Pdcd6, Pdcl, Pdcl3,
Pde1a, Pde2a, Pde4dip, Pde6a, Pde7a, Pdgfa, Pdha1, Pdia3, Pdia6, Pdk4, Pdlim4, Pdlim5, Pds5b, Pdss1, Pdxk,
Pdzd11, Pecam1, Pef1, Per1, Per3, Perp, Pex11c, Pex12, Pex19, Pex5, Pex6, Pex7, Pfdn1, Pfdn4, Pfdn5, Pfkp, Pfn2,
Pgam2, Pgbd5, Pggt1b, Pgr, Phc2, Phc3, Phf14, Phf17, Phf20I1, Phf3, Phf6, Phka2, Phkb, Phkg1, Phlda1 , Phldb1,
Phpt1, Phyh, Pias4, Picalm, Pigz, Pik3ca, Pik3ip1, Pip5k1c, Pir, Pitpna, Pitpnb, Pitpnc1, Pitpnm1, Pkd1, Pkia, Pkm2,
Pkp2, Pla2g10, Pla2g2d, Pla2g5, Plcd1, Plce1, Pld3, Pldn, Plec1, Plekhh3, Plekhj1, Plekhm2, Plekhn1, Plod3, Plp2, Pls3,
Pltp, Plvap, Plxnb2, Pmm1, Pno1, Pnpla1, Pnpla2, Pnpla6, Pnrc2, Podn, Poldip3, Polg2, Polr2d, Polr2f, Polr2h, Polr2i,
Polr2k, Polr3gl, Polr3k, Pot1a, Pou6f1, Ppap2b, Ppard, Pparg, Ppargc1a, Pphln1 , Ppic, Ppif, Ppig, Ppil2, Ppl, Ppm1f,
Ppme1 , Ppp1ca, Ppp1r11, Ppp1r12a, Ppp1r12c, Ppp1r13l, Ppp1r2, Ppp1r3c, Ppp2ca, Ppp2r2d, Ppp2r3c, Ppp2r5c,
Ppp4r1l, Ppp5c, Ppp6c, Ppt2, Pqlc1, Prdm4, Prdm5, Prdx2, Prdx3, Preb, Prei4, Prkab2, Prkaca, Prkcbp1, Prkcdbp,
Prkch, Prkcn, Prkcsh, Prkcz, Prkrir, Prlr, Prmt7, Prodh, Prosc, Prpf6, Prpsap1, Prr12, Prrc1, Prss12, Prune, Psap, Pscd1,
Psd3, Psenen, Pskh1, Psma2, Psma5, Psma6, Psma8, Psmb7, Psmd11, Psmd12, Psmd4, Psmd6, Psmd8, Pstk, Ptdss2,
Ptgfrn, Ptms, Ptp4a3, Ptpla, Ptpn1, Ptpn11, Ptpn12, Ptpn20, Ptpn3, Ptpra, Ptprg, Ptprs, Pttg1, Puf60, Pum1, Pus1,
Pxmp3, Pxn, Qk, Rab1, Rab11a, Rab11b, Rab2, Rab20, Rab21, Rab24, Rab30, Rab33b, Rab35, Rab3a, Rab3gap1,
Rab3gap2, Rab3il1, Rab43, Rab6, Rab8b, Rabep1, Rabgap1l, Rac1, Rad17, Rad23b, Rad54l2, Rag1ap1, Ralgps2,
Ramp2, Ranbp10, Ranbp2, Rap1a, Rap1gap, Rap2a, Rap2b, Raph1, Rara, Rarb, Rarg, Rasa1, Rasa3, Rasl2-9,
Rassf7, Rb1cc1, Rbbp6, Rbj, Rbm12, Rbm20, Rbm24, Rbm27, Rbm28, Rbm38, Rbm39, Rbms1, Rbms2, Rbmxrt,
Rbpms, Rcan2, Rcl1, Rdh13, Reep5, Rem2, Retsat, Rev1, Rfc2, Rfc4, Rfesd, Rfng, Rfwd3, Rfx1, Rgl1, Rgma, Rgs12,
Rgs5, Rhbdd2, Rhbdd3, Rhbdf1, Rhd, Rhobtb1, Rhobtb2, Rhoq, Ric8, Ring1, Rlbp1, Rmnd1, Rmnd5b, Rnaset2a,
Rnd1, Rnf11, Rnf13, Rnf139, Rnf14, Rnf149, Rnf167, Rnf168, Rnf187, Rnf2, Rnf31, Rnf34, Rnf5, Rnf6, Rock1, Rorc, RP23-
136K12.4, rp9, Rpap2, Rpe, Rpl15, Rpl27a, Rpl37, Rpl39, Rpl31, Rpl711, Rpl8, Rpip2, Rpol-3, Rpol-4, Rpp30, Rprml,
Rps11, Rps26, Rps6, Rps6ka1, Rps6ka4, Rrad, Rragc, Rragd, Rras2, Rrbp1, Rrp1, Rrp9, Rsad1, Rspry1, Rtp3, Rufy1,
Rufy3, Rusc1, Rwdd1, Rxrb, Rxrg, Ryk, Ryr2, S3-12, Sae2, Safb, Samd5, Samd8, Samd9l, Saps3, Sar1a, Sars, Sat1,
Satb2, Sbds, Sbf2, Sbk1, Sc4mol, Scamp3, Scap, Scara5, Scarb1, Scarb2, Sccpdh, Scfd1, Schip1, Scmh1, Scn4b,
Scoc, Scube2, Scyl1, Scyl3, Sdcbp, Sdccag10, Sdha, Sdhd, Sds, Sec11a, Sec14l1, Sec22b, Sec23a, Sec31a,
Sec61a1, Sec61a2, Sele, Sema3b, Sephs2, Sepp1, Serbp1, Serinc1, Serpinb6a, Serpinb9, Sertad2, Set, Setd7, Setd8,
Setx, Sf3a1, Sf3b1, Sf3b2, Sfrp5, Sfrs1, Sfrs10, Sfrs2ip, Sfrs7, Sfrs9, Sfxn3, Sgca, Sgcg, Sgk2, Sgta, Sh2d3c, Sh2d4a,
Sh3bgrl, Sh3bp5l, Sh3d19, Sh3kbp1, Shb, Shmt2, Shroom3, Sirt1, Skit, Skiv2l2, Slain2, Slc10a1, Slc12a4, Slc12a5,
Slc16a1, Slc16a4, Slc1a5, Slc1a6, Slc20a1, Slc22a17, Slc22a5, Slc25a11, Slc25a12, Slc25a17, Slc25a22, Slc25a28,
Slc25a3, Slc25a32, Slc25a33, Slc25a34, Slc25a36, Slc25a4, Slc25a42, Slc25a46, Slc26a11, Slc27a1, Slc29a1, Slc31a1,
Slc35a2, Slc35a3, Slc35b1, Slc35b2, Slc36a2, Slc39a1, Slc39a10, Slc39a8, Slc40a1, Slc44a1, Slc47a1, Slc4a2, Slc4a4,
Slc4a7, Slc6a19, Slc6a6, Slc6a9, Slc7a1, Slc7a4, Slc7a7, Slc9a1, Slco1a4, Slco3a1, Slco5a1, Slit3, Slmap, Slmo2,
Smarca2, Smarcc2, Smarcd3, Smchd1, Smcr7, Smn1, Smndc1, Smoc2, Smpd1, Smpdl3a, Smtn, Smtnl2, Smu1,
Smurf1, Smyd1, Smyd4, Snapap, Snapc1 , Snf1lk2, Snora65, Snrk, Snrp70, Snrpb2, Snrpd3, Snx12, Snx13, Snx16, Socs3,
Socs4, Sorbs1, Sorcs2, Sort1, Sost, Sox17, Sox4, Sox9, Sp3, Spag9, Spcs1, Speer7-ps1, Spg3a, Spg7, Spin1, Spink10,
Spna2, Spnb1, Spnb2, Spop, Spry2, Sqstm1, Srd5a2l2, Srebf1, Srebf2, Sri, Sri, Srp19, Srpr, Ssbp3, Ssbp4, Ssh1, Ssr3, Sstr5,
Ssu72, St13, St3gal6, St6galnac6, St7, St8sia2, St8sia4, Stab1, Stard10, Stat6, Stbd1, Stch, Stip1 , Stk11, Stk19, Stk38,
Stk39, Stom, Strn3, Stx6, Stxbp2, Styx, Suhw3, Sulf2, Supt5h, Supv3l1, Surf4, Svep1, Sybl1, Syk, Syn2, Syngr2, Synj2bp,
Synpo, Sypl, Taf2, Tato, Tanc1, Taok2, Taok3, Tap1, Tap2, Tapt1, Tardbp, Tatdn3, Tbc1d10b, Tbc1d15, Tbc1d19,
Tbc1d20, Tbc1d2b, Tbc1d5, Tbc1d7, Tbcb, Tbcc, Tbce, Tbcel, Tbkbp1, Tbpl1, Tbx19, Tbx20, Tcap, Tcea1, Tcea3,
Tcf15, Tcf20, Tcf25, Tcfe2a, Tcof1, Tcp1, Tcp11l2, Tcta, Tead4, Tef, Tesk1, Tex2, Tex261, Tfam, Tfb2m, Tfpi, Tfrc, Tgds,
Tgfbr1, Thbs4, Thnsl2, Thocl, Thoc4, Thrb, Tie1, Tigd2, Timm22, Timm50, Timp3, Timp4, Tinagl, Tjap1, Tk2, Tle6, Tlk2, Tln1,
Tloc1, Tm2d1, Tm2d2, Tm2d3, Tm4sf1, Tm6sf2, Tm9sf2, Tm9sf3, Tmc1, Tmcc1, Tmcc3, Tmco1, Tmed7, Tmem103,
Tmem109, Tmem110, Tmem112b, Tmem115, Tmem119, Tmeml23, Tmem126b, Tmem132a, Tmem142a, Tmem142c,
Tmem147, Tmem14c, Tmem157, Tmem159, Tmem167, Tmem168, Tmem16f, Tmem176a, Tmem176b, Tmem182,
Tmem188, Tmem19, Tmem30a, Tmem37, Tmem38a, Tmem38b, Tmem41a, Tmem41b, Tmem46, Tmem50a,
Tmem55b, Tmem57, Tmem64, Tmem69, Tmem70, Tmem77, Tmem85, Tmem86a, Tmem93, Tmem9b, Tmlhe, Tmod1,
Tmod4, Tmub1, Tmub2, Tnfaip1, Tnfaip8l1, Tnfrsf11a, Tnfrsf18, Tnfrsf1a, Tnfsf5ip1, Tnip1, Tnks1bp1, Tnni3, Tnni3k, Tnnt2,
Tnpo1, Tnpo2, Tnrc6a, Tns4, Tnxb, Toe1, Tollip, Tomm22, Tomm34, Tomm40, Tomm70a, Top1, Top2b, Topors, Tor1aip2,
Tpcn1, Tpm1, Tpm3, Tpm4, Tpp1, Tpp2, Tppp3, Tpr, Tprkb, Tpst1, Traf3ip2, Traip, Trak2, Tra ppc2, Trappc2l, Trem3,
Trex1, Trim11, Trim12, Trim23, Trim26, Trim29, Trim3, Triobp, Trip4, Trmt11, Tro, Troap, Trpc1, Trpc4ap, Trpm4, Tsc22d1,
Tsc22d4, Tsfm, Tsga10, Tsnax, Tspan13, Tspan18, Tspan4, Tspan7, Tssc4, Tsta3, Ttc1, Ttc28, Ttc32, Ttc33, Ttc35, Ttc9c,
Tub, Tuba4a, Tuba8, Tubb2c, Tubb5, Tufm, Tug1, Tulp4, Twsg1, Txlna, Txlnb, Txndc1, Txndc10, Txndc12, Txndc14,
Txndc4, Txnip, Txnl1, Txnl4, Txnl4b, Tyk2, Uaca, Ubac1, Ubap1, Ubash3a, Ubd, Ube1c, Ube1l2, Ube1x, Ube2b,
Ube2d2, Ube2d3, Ube2e1, Ube2f, Ube2h, Ube2n, Ube2o, Ube2q2, Ube2v1, Ube2v2, Ube2w, Ube3a, Ubl4, Ubl7,
Ublcp1, Ubtf, Ubxd7, Uchl5, Ucp2, Ucp3, Ufm1, Ugcgl2, Ugp2, Umps, Ung, Unk, Uqcc, Uqcrc1, Uqcrfs1, Usp11, Usp19,
Usp2, Usp21, Usp22, Usp34, Usp36, Usp45, Usp47, Usp52, Usp54, Usp9y, Utp6, Utrn, Uvrag, Uxt, V1ra5, Vamp4, Vasn,
Vbp1, Vdac1, Vdac2, Vdac3, Vdp, Vegfa, Vegfb, Vegfc, Vezf1, Vkorc1l1, Vldlr, Vps16, Vps18, Vps29, Vps35, Vps36,
Vps37b, Vps4b, Vps54, Vwf, Wac, Wa pal, Was, Wbp4, Wdfy1, Wdr13, Wdr21, Wdr22, Wdr23, Wdr3, Wdr47, Wdr5b,
Wdr92, Wdsof1, Wfdc3, Wipi2, Wnk1, Wtap, Wwp2, Xbp1, Xdh, Xlr5a, Xpnpep1, Xpr1, Xrcc1, Xrcc6, Yap1, Yeats2,
Yif1a, Yipf3, Yipf4, Yipf7, Ypel2, Ypel3, Ywhaq, Zadh1, Zbed3, Zbtb43, Zbtb5, Zc3h11a, Zc3h12c, Zc3h15, Zc3h6,
Zc3h8, Zcchc6, Zdhhc13, Zdhhc3, Zeb1, Zfand5, Zfml, Zfp106, Zfp110, Zfp187, Zfp191, Zfp213, Zfp236, Zfp238, Zfp26,
Zfp260, Zfp277, Zfp289, Zfp30, Zfp313, Zfp319, Zfp322a, Zfp335, Zfp341, Zfp35, Zfp383, Zfp384, Zfp414, Zfp422, Zfp422-
rs1, Zfp512, Zfp516, Zfp560, Zfp568, Zfp579, Zfp597, Zfp608, Zfp628, Zfp629, Zfp639, Zfp644, Zfp650, Zfp651, Zfp667,
Zfp672, Zfp68, Zfp703, Zfp715, Zfp719, Zfp740, Zfp758, Zfp817, Zfp82, Zfyve21, Zhx2, Zhx3, Zic2, Zkscan17, Zmat2,
Zmat5, Zmym4, Zmynd10, Znrf1, Zrsr1, Zscan12, Zswim6, Zyg11b, Zyx, Zzef1

TABLE 2
Exemplary Genes Whose Expression Enhances Cellular Damage

AA407175, AA415038, AA987161, Aadacl1, Aars, Aasdhppt, AB182283, Abca4, Abca7, Abcb4, Abcb7, Abcd1,
Abce1, Abhd1, Abhd12, Abhd4, Abi1, Abi2, Ablim2, Abra, Abtb1, Acaa2, Acad11, Acad9, Acadl, Acads, Acadvl,
Acbd3, Acbd5, Acbd6, Ace, Aco2, Acot5, Acox3, Acsl1, Acss2, Acta1, Actb, Actn1, Actn2, Actn4, Actr1b, Actr2,
Acvr1b, Acvr2a, Acvrl1, Acyp1, Acyp2, Adal, Adam10, Adam15, Adam21, Adamts10, Adamts2, Adamts7,
Adamts9, Adar, Adcy1, Adcy2, Adcy3, Adcy6, Add1, Adh5, Adra1b, Adrbk1, Aebp1, Aes, Afap1l1, Aff4, Afg3l1,
Aga, Agbl5, Agpat1, Agpat5, Agrn, Agtr1a, Agxt2l2, Ahdc1, Ahr, Ahsa1, Al118078, Al225934, Al413194, Al428479,
Al429363, Al462493, Al480535, Al506816, Al597468, Al662270, Al662476, Al747699, Al790298, Al837181, Al848100,
Al852064, Al987944, Ak7, Akap13, Akap2, Akp2, Akr1a4, Akr1b8, Akr7a5, Akt1s1, Alas1, Aldh1a3, AIdh2, Aldh4a1,
Aldh7a1, Aldh9a1, Alg12, Alg13, Alg5, Alkbh6, Alkbh8, Als2cr2, Anapc10, Anapc2, Angptl2, Ank1, Ankhd1, Ankrd1,
Ankrd10, Ankrd13a, Ankrd13c, Ankrd13d, Ankrd25, Ankrd28, Ankrd32, Ankrd37, Ankrd38, Ankrd9, Anp32b, Anxa3,
Anxa6, Aoc3, Ap1s2, Ap2a2, Ap2b1, Ap2m1, Ap4m1, Ap4s1, Apbb1, Aplp2, Apobec2, Apod, Apoe, Apool, Appl1,
Appl2, Arf1, Arf3, Arg2, Arid4b, Arl1, Arl2bp, Arl3, Arl4a, Arl5b, Arpc1a, Arpc1b, Arpc2, Arpc4, Arrb1, Art5, Asb1,
Asb14, Asb5, Ascc3l1, Asnsd1, Asph, Atad2b, Atf3, Atg10, Atg3, Atg4d, Atg5, Atp11b, Atp13a1, Atp1a2, Atp5h,
Atp5s, Atp6ap2, Atp6v0a2, Atp6v0d1, Atp6v1b2, Atp6v1f, Atp9a, Atp9b, Atpaf1, Atpbd1c, Atpif1, Atr, Atxn2,
Atxn7l1, AU020772, AU041133, Aup1, AV009015, AV024533, AV025504, Avpr1a, AW046287, AW112010, AW209491,
AW555464, AW556556, AW742931, Azi2, Azin1, Bach1, Bag4, Bambi, Banp, Bat1a, Bat2, Baz1a, Baz1b, BB217526,
Bbc3, Bbs10, BC003331, BC003885, BC003965, BC010304, BC010981, BC011248, BC013529, BC016495, BC019943,
BC020077, BC021395, BC023882, BC024659, BC024814, BC025076, BC028440, BC028528, BC030183, BC030308,
BC030336, BC031353, BC031781, BC032203, BC034069, BC037034, BC037112, BC038479, BC039210, BC043098,
BC043476, BC048679, BC049349, BC057893, Bcam, Bcat2, Bckdha, Bckdk, Bcl2l13, Bcl6b, Bclaf1, Bdp1, Bet1, Bgn,
Bhlhb2, Bhlhb3, Bicd2, Birc4, Blvra, Bmi1, Bmp6, Bmpr1a, Bnip3, Brd3, Btaf1, Btbd14b, Btbd2, Btbd3, Btbd6, Btf3l4,
Btnl9, Bxdc2, C130094E24, C2, C77058, C78441, C78651, C79741, C86942, C87259, Cab39, Cabin1, Cacna1g,
Cacna1h, Cacybp, Cadm4, Calm1, Calr, Calr3, Caml, Camsap1, Cand2, Canx, Capg, Capn1, Capns1, Caprin1,
Card10, Caskin2, Casp8ap2, Casp9, Casq1, Cav1, Cav2, Cbfb, Cblb, Cbr1, Cbx3, Ccar1, Ccdc12, Ccdc122,
Ccdc125, Ccdc127, Ccdc3, Ccdc34, Ccdc47, Ccdc58, Ccdc69, Ccdc7, Ccdc72, Ccdc85b, Ccdc88a,
Ccdc90a, Ccdc90b, Ccl9, Ccm2, Ccnd3, Ccng1, Ccnh, Ccni, Ccnl2, Ccnt2, Ccr1, Ccr1l1, Ccr5, Ccs, Cct5, Cct7,
Cd151, Cd163, Cd200, Cd207, Cd36, Cd38, Cd74, Cd83, Cd93, Cd97, Cdadc1, Cdc27, Cdc2l5, Cdc2l6, Cdc37,
Cdc42ep3, Cdgap, Cdh13, Cdipt, Cdk5rap3, Cdk7, Cdv3, Cebpz, Cenpa, Cenpq, Cental, Centa2, Centb2,
Centd1, Centd2, Centg2, Cetn3, Cfl1, Cflar, Cgnl1, Cgrrf1, Chac1, Chac2, Chchd4, Chd1, Chd2, Chd4, Chmp1b,
Chmp2b, Chordc1, Chrac1, Chrd, Chrng, Chst14, Chuk, Churc1, Ciao1, Cib1, Cic, Cilp2, Cisd2, Cish, Ckm, Ckmt2,
CIcn5, Cldnd1, Clec2d, Clic1, Clic4, Clint1, Clk3, Cln5, Clock, Clptm1, Clstn1, Cltc, Cmpk, Cmya5, Cndp2, Cnot6l,
Cnot7, Cntfr, Cntn4, Commd1, Commd3, Commd4, Commd5, Comp, Cope, Copg, Cops2, Cops7a, Coq10b,
Coq9, Coro1b, Cox11, Cox4i2, Cox5a, Cox8a, Cp, Cpeb4, Cpm, Cpsf1, Cpsf3, Cpt1a, Cpt1b, Cramp1l, Crat,
Crbn, Creb1, Creb3l1, Crebbp, Crebzf, Creg1, Crip1, Crip2, Cript, Crnkl1, Crot, Cry1, Cryab, Crybb1, Cryz, Csdc2,
Cse1l, Csf2ra, Csl, Csnk1a1, Csnk1d, Csnk2a1, Cst3, Cst8, Cstf2, Ctage5, Ctcf, Ctgf, Ctps, Ctsb, Ctsf, Ctss, Ctsz,
Cttnbp2nl, Cul1, Cul3, Cxcl12, Cxcl14, Cxxc1, Cxxc5, Cyb561, Cyb5b, Cyb5r3, Cyb5r4, Cybasc3, Cyc1, Cyfip1,
Cyp1b1, Cyp27a1, Cyp2f2, Cys1, D030063E12, D0H4S114, D10Ertd641e, D13Ertd787e, D14Ertd16e, D14Ertd581e,
D15Ertd50e, D16H22S680E, D19Ertd721e, D19Ertd737e, D19Wsu162e, D1Bwg1363e, D2Ertd391e, D3Ertd254e,
D3Wsu106e, D4Ertd429e, D4Ertd571e, D6Wsu176e, D8Ertd457e, D8Ertd54e, D8Ertd620e, D8Ertd82e, D9Ertd402e,
Daam1, Dad1, Dap, Dapk2, Daxx, Dbh, Dcn, Dctn1, Dctn2, Dcun1d1, Dcun1d2, Dcun1d5, Ddah2, Ddb1, Ddb2,
Ddit3, Ddr1, Ddr2, Ddx1, Ddx17, Ddx39, Ddx51, Ddx54, Ddx58, Ddx6, Deb1, Dedd, Defb1, Defb5, Defcr15, Depdc7,
Derl2, Des, Dfna5h, Dgat2, Dgcr2, Dgka, Dgke, Dguok, Dhodh, Dhrs1, Dhrs7, Dhx30, Dhx32, Dhx34, Dhx8, Dhx9,
Diablo, Diap1, Diras1, Dirc2, Dkk3, Dld, Dll4, Dist, Dmd, Dmpk, Dmtf1, Dmwd, Dmxl2, Dnahc9, Dnaja3, Dnajb1,
Dnajb4, Dnajb9, Dnajc12, Dnajc3a, Dnajc7, Dnm2, Dock11, Dock6, Dom3z, Dopey1, Dot1l, Dpagt1, Dph3, Dpp8,
Dpp9, Dpysl2, Dpysl3, Dr1, Drg1, Dstn, Dtnbp1, Dus31, Dusp1, Dusp6, Dusp8, Dvl2, Dync1h1, Dync1li2, Dyrk1a, E2f6,
Eaf1, Eapp, Ears2, Ebag9, Ece1, Ecm1, Ecm2, Edaradd, Edg3, Eea1, Eef1a1, Eef1b2, Eef1e1, Eef2, Efcab2, Efemp2,
Efnb3, Egf, Egfl7, Egflam, Egfr, Egln1, Egln3, Egr1, Ehbp1l1, Ehd4, Ei24, Eif1ay, Eif2ak1, Eif2s2, Eif3e, Eif4a1, Eif4a2,
Eif4b, Eif4e2, Eif4ebp1, Eif4g3, Eif5, Eif5b, Elac2, Elf2, Elk3, Ell, Ell2, Elovl5, Elp3, Elp4, Eltd1, Emb, Emd, Eme2, Emg1,
Emilin1, Eml2, Enc1, Eng, Eno3, Enpep, Enpp5, Entpd5, Entpd6, Ep300, Epb4.1l3, Epha4, Ephb1, Ephb4, Epm2aip1,
Epn1, Eps15l1, Erc1, Ergic3, Erlin1, Ero1lb, Errfi1, Esco1, Esd, Esf1, Esrrg, Etfa, Etnk1, Ets2, Ewsr1, Exoc5, Exosc1, Exosc10,
Exosc7, Exosc9, Ext1, Eya3, F11r, F13b, F5, Fads3, Fand2a, Fam18b, Fancg, Fap, Fas, Fastkd1, Fastkd2, Fbln1, Fbln2,
Fbp2, Fbxl2, Fbxl6, Fbxo3, Fbxo30, Fbxw4, Fbxw5, Fcer2a, Fcgr4, Fdx1, Fem1c, Fert2, Fgfr1op, Filip1, Fkbp10, Fkbp5,
Fkbp8, Flcn, Flii, Flot1, Flot2, Flywch1, Fmn1, Fmo2, Fmr1, Fnbp1l, Fnip1, Foxa3, Foxj2, Foxk1, Foxk2, Foxo1, Foxp1,
Frag1, Frap1, Frmd4b, Frmd5, Fscn1, Fth1, Ftl1, Fuca2, Fundc2, Furin, Fus, Fxc1, Fxyd1, Fxyd5, Fzd10, Fzd2, Fzd9,
G0s2, G6pc2, Gaa, Gab1, Gabpa, Gadd45b, Gadd45g, Gale, Galk1, Gapdh, Gapvd1, Garnl1, Gas6, Gata4,
Gba2, Gbas, Gbe1, Gbf1, Gcdh, Gdi1, Gdi2, Gdpd1, Gdpd5, Gemin5, Ggta1, Ghitm, Gimap4, Gimap8, Git1,
Gja3, Gle1l, Glg1, Gli1, Glo1, Glod4, Gls, Gltscr2, Glud1, Glul, Gm104, Gm561, Gmeb1, Gmfb, Gmppa, Gna-rs1,
Gnb2, Gnb4, Gne, Gng10, Gn13, Gnpda1, Golga2, Golga7, Golgb1, Got1, Got2, Gpaa1, Gpam, Gpatch1,
Gpbp1, Gpbp1l1, Gpc1, Gpc6, Gpd1l, Gper, Gpkow, Gpr115, Gpr137, Gpr175, Gpr22, Gpr4, Gpr98, Gpsn2, Gpt2,
Gpx3, Gramd1a, Grb14, Grina, Grk1, Grk5, Grilf`, Grm8, Grn, Grpel2, Gsdmdc1, Gsn, Gsta4, Gstcd, Gstm1, Gstm2,
Gstm5, Gstm7, Gstp1, Gstt1, Gtf2a1, Gtf2a2, Gtf2e1, Gtf2e2, Gtf2h3, Gtf2h4, Gtf3c1, Gtf3c4, Gtpbp1, Gtpbp2,
Gulo, Gyg, Gyk, Gys1, H2afv, H2afy, H2-Bl, H2-Oa, H2-T24, H6pd, Hadh, Hadha, Hadhb, Hand2, Hars, Hars2, Hat1,
Hax1, Hccs, Hcfc1r1, Hcfc2, Hdac2, Hdac4, Hdac7a, Hdhd2, Hdlbp, Heatr5b, Heatr6, Hectd1, Heph, Herpud1,
Heyl, Hfe2, Hgs, Hhatl, Hiat1, Hiatl1, Hibadh, Hif1a, Higd1b, Hint3, Hirip3, Hivep2, Hk3, Hlf, Hmcn1, Hmg20b, Hmgb1,
Hmgb3, Hmgcl, Hmgcs1, Hnrpab, Hnrph3, Hnrpk, Hnrpl, Hnrpll, Hnrpr, Hnrpul1, Hoxd11, Hrasls, Hrc, Hs2st1,
Hsd17b11, Hsd17b13, Hsd17b4, Hsdl2, Hsp110, Hspa1b, Hspa5, Hspb2, Hspb3, Hspb6, Hspb7, Hspe1, Htra1, Htra3,
Hus1, Hyal4, Iah1, Ibrdc2, Id1, Ide, Idh3a, Idh3b, Ifi30, Ifit3, Ifnar1, Ifnar2, Ifngr1, Ifngr2, Ift122, Ift57, Igfbp4, Igfbp6,
Igsf11, Igsf3, Igsf8, Ihpk1, Ikbkap, Il10rb,Il13ra1, Il18bp, Il6st, Ilk, Ilvbl, Immp1l, Immp2l, Immt, Imp3, Impa2, Impad1,
Ints8, Ipmk, Ipo13, Ipo7, Ipo8, Iqsec1, Iqwd1, Irf4, Irs1, Isca2, Isg20, Isyna1, Itfg3, Itgb1bp1, Itgb1bp2, Itgb1bp3, Itgb2,
Itgb5, Itih3, Itk, Itm2b, Itm2c, Itpr3, Ivns1abp, Jam2, Jmjd1c, Jmjd2a, Jmjd6, Josd2, Jtv1, Jun, Kbtbd10, Kbtbd5,
Kcnip2, Khk, Kif1b, Kif1c, Kif21a, Kif2a, Kif3a, Kif5b, Klc3, Klf11, Klf13, Klf15, Klf16, Klf4, Klf7, Klhdc1, Klhdc3, K1hl13,
Klhl22, Klhl23, Klhl24, Klhl4, Klhl9, Klk1b24, Kpna1, Kpna4, Krr1, Kti12, Ktn1, L1cam, I7Rn6, Lace1, Lactb, Lama2,
Lamb2, Laptm4a, Larp1, Larp2, Larp4, Larp5, Lcmt1, Lcmt2, Ldb1, Ldb3, Ldhb, Ldhd, Leo1, Lgals3bp, Lgals7, Lgmn,
Lgr4, Lgr6, Lias, Limd1, Lims2, Lipe, Lix1l, Llgl1, Lmbrd1, Lmln, Lmna, Lmo4, Lmtk2, LOC100040515, LOC100043489,
LOC100046468, LOC100046982, LOC552902, Lonp1, Lor, Lpgat1, Lphn1, Lrch1, Lrch4, Lrp10, Lrp2bp, Lrp6, Lrpap1,
Lrrc1, Lrrc20, Lrrc39, Lrrc3b, Lrrc40, Lrrc44, Lsm14a, Lsm14b, Lsm3, Ltb4dh, Ltbp3, Ltbp4, Ly6a, Lypla1, Lypla2, Lyrm4,
Lyrm5, Lysmd2, Lysmd3, Lztfl1, Lztr1, M6prbp1, Macf1, Macrod1, Maf1, Magea5, Magee1, Magi3, Mall, Man2b1,
Maob, Map1lc3a, Map1lc3b, Map2k1ip1, Map2k2, Map3k1, Map3k12, Map3k2, Map3k7, Map3k7ip1, Map4k5,
Mapbpip, Mapk14, Mapk6, Mapkapk2, Mapre1, Marcks, Mat2a, Mat2b, Matn4, Maz, Mbc2, Mbd2, Mbd3, Mbd5,
Mboat5, Mbtps1, Mbtps2, Mcf2l, Mctp2, Mdfic, Mdh2, Med13, Med16, Medl9, Med25, Med30, Med7, Mef2b,
Mef2c, Mef2d, Megf11, Megf8, Mel13, Mertk, Mesdc2, Metrnl, Mett10d, Mex3c, Mfap4, Mfge8, Mfn1, Mfsd8,
Mgam, Mgat1, Mgat4b, Mgp, Mgrn1, Mif4gd, Mkl1, Mknk1, Mlf1, Mlkl, Mll2, Mllt1, Mllt6, Mlx, Mlxip, Mlycd, Mme,
Mmp15, Mmp1b, Mmp2, Mmrn2, Mobkl3, Mocos, Mocs2, Morc2a, Mosc2, Mospd1, Mpa2l, Mpp6, Mpv17, Mrfap1,
Mrgprf, Mrpl1, Mrpl15, Mrpl17, Mrpl19, Mrpl28, Mrpl30, Mrpl32, Mrpl36, Mrpl38, Mrpl4, Mrpl41, Mrps17, Mrps18c,
Mrps22, Mrps5, Mrs2l, Msl31, Msra, Msrb2, Msrb3, Msx1, Mtap4, Mtap7d1, Mtbp, Mtch2, Mterf, Mterfd1, Mterfd2,
Mterfd3, Mtif3, Mtmr1, Mtmr3, Mtrr, Mustn1, Mxd4, Mxi1, Mxra8, Mybbp1a, Mybpc3, Myc, Mycbp, Myct1, Myd116,
Myd88, Myef2, Myh14, Myh6, Myl4, Myl7, Mylip, Myo10, Myo1c, Myo9b, Myocd, Myom1, Mypn, N6amt1, N6amt2,
Naca, Nagpa, Nars, Nat5, Nbeal1, Nckap1l, Ndst4, Ndufa5, Ndufab1, Ndufaf1, Ndufb3, Ndufb7, Ndufb8, Ndufc1,
Ndufc2, Ndufs1, Ndufs2, Ndufs3, Ndufs5, Ndufs7, Ndufs8, Ndufv1, Ndufv2, Nedd4, Neil1, Nek3, Nf2, Nfu1, Ngly1,
Ngrn, Nid1, Nif3l1, Ninj1, Nipbl, Nkiras1, Nkiras2, Nlgn2, Nmnat1, Npc1, Nppb, Nras, Nrd1, Nrp1, Nrp2, Nrtn, Nsmaf,
Nt5c2, Nt5c3, Nub1, Nwd1, Oasl2, Ogg1, Oplah, Pfkfb2, Pfkfb4, Pgc1, Pgls, Pygb, Rars, Rars2, Rere, Rnpepl1, RP23-
233B9.8, Rsrc2, Smad1, Smad3, Smad6, Ucp3, X83328, Xk, Xpo4, Xpo6, Xpot, Xrn1

Some embodiments provide a composition that comprises trans-resveratrol and a metal chelating agent, and may additionally comprise quercetin, one or more glycosaminoglycans, and/or vitamin D. The trans-resveratrol may be encapsulated to substantially preserve the biological activity of the composition from loss due to exposure of the trans-resveratrol to light or oxygen. Particularly provided are compositions that comprise resveratrol, a chelator, hyaluronic acid, and/or vitamin D, and compositions which comprise the chelator phytic acid (inositol hexaphosphate; IP6), the glycosaminoglycan hyaluronic acid, and vitamin D.

Other embodiments provide resveratrol-containing compositions capable of modulating gene expression to an extent greater than that observed with resveratrol alone or with calorie restriction. The compositions may be used to up-regulate a survival/longevity gene or down-regulate a gene whose expression enhances cellular damage upon administration to a recipient, and may also be used in the treatment or prevention of cancer, cardiovascular disease, diseases associated with aging, and other conditions and illnesses. Particular embodiments provide a resveratrol-containing composition that, upon administration to a recipient, modulates the concentration or activity, relative to resveratrol alone or calorie restriction, of the product of a survival/longevity gene or the product of a gene whose expression enhances cellular damage. Administration is preferably by oral ingestion.

The embodiments further particularly pertains to compositions that increase the concentration of the forkhead Foxo1 (daf-16, dFoxO) transcription factor survival/longevity gene product.

Particular embodiments provide compositions and methods where the modulation alters: (A) oxidative phosphorylation; (B) actin filament length or polymerization; (C) intracellular transport; (D) organelle biogenesis; (E) insulin signaling; (F) glycolysis; (G) gluconeogenesis; or (H) fatty acid metabolism. The gene product may be a survival/longevity gene product, and particularly Sirtuin 1, Sirtuin 3, or the forkhead Foxo1 transcription factor. The gene product may enhance cellular damage, and particularly may be encoded by the uncoupling protein 3, Pgc-1, or pyruvate dehydrogenase kinase 4 genes.

### D. Packaging of the Compositions

Resveratrol is typically unstable to light and oxidation (Shaanxi University of Science & Technology, Xianyang China (2007) Zhong Yao Cai. 30 (7):805-80). The resveratrol of the present embodiments is preferably prepared, packaged and/or stored in a manner that maximizes its specific activity. It is preferred to prepare, package and/or store resveratrol in low light (or in the dark) and/or in low oxygen, so as to minimize light-induced degradation (e.g., photo-isomerization) or oxygen-induced degradation. The preferred compositions of the present embodiments are formulated as dietary supplements for oral ingestion in the form of a pill, lozenge, capsule, elixir, syrup, etc. Other modalities of administration may alternatively be employed (e.g., intranasal, parenteral, intravenous, intraarterial, topical, etc.).

The resveratrol or plant extract comprising resveratrol is preferably encapsulated in a substantially oxygen-free environment. As used herein, the phrase “substantially oxygen-free” is intended to include environments having less than less than about 100 parts per million oxygen. Ideally, the encapsulation process would take place immediately after the extraction or formation of the small molecules and be shielded from exposure to light, heat, and oxygen. Alternatively, the material including small molecules may be stored in a substantially oxygen-free environment until encapsulated. The encapsulation process includes the steps of (1) providing a capsule including a head portion and a body portion; (2) at least partially filling the body portion with the material including biologically active small molecules; (3) axially positioning the head portion over the body portion such that the portions at least partially overlap; and (4) forming a fluid tight (air and liquid impermeable) seal along the overlapping portions.

The material comprising the capsule portions is not particularly limited. Preferably, the capsule portions comprise material possessing a low oxygen transmission rate. For example, it is preferred the capsule portions comprise a material having an oxygen transmission rate (as measured by ASTM D3985) of less than about 165 cm³/m²/day for 100 μm, more preferably less than about 4 cm³/m²/day for 100 μm, and most preferably less than about 1 cm³/m²/day for 100 μm. Exemplary materials comprising the capsule portions include, but are not limited to, an ingestible material such as gelatin, hydroxypropyl methylcellulose, or starch. By way of specific example, the material may include gelatin having an oxygen transmission rate of about 3.5 cm³/m²/day for 100 μm. The resulting capsules may include hard gelatin capsules or soft gelatin capsules having an oxygen transmission rate of up to about 0.04 cm³/capsule/day (ASTM D3985 at 27° C. and rel. humidity of 50%). In addition, opaque capsules are highly preferred. This can be achieved by adding pigment such as titanium dioxide to the capsule material formulation. Titanium dioxide is inert and possesses a high molecular weight, which prevents it from being absorbed into blood circulation when ingested. Opaque capsules function to prevent the degradation of the resveratrol-containing composition by light degenerative processes such as photooxidation. A commercially available, opaque capsule having low oxygen permeability is available from Capsugel (Greenwood, S.C.—www.capsugel.com), sold under the trade name Licaps®.

The system used to encapsulate the composition including biologically active small molecules material must create a fluid-tight (air and liquid impermeable) seal around capsule portions. A particularly preferred encapsulation system and process is disclosed in WO 01/08631A1, incorporated herein by reference in its entirety. In this system and associated process, a capsule head portion and a capsule body portion are placed in a filling chamber. The capsule body portion is filled with the desired dosage material, and the capsule portions are then telescopically joined such that the head portion partially overlaps the body portion. A sealing liquid including a solvent is applied in the gap formed between the overlapping sections, and the capsule is dried to remove the solvent and form a fluid-tight seal.

It is important that the encapsulation process occurs in a substantially oxygen-free environment. In addition, it is preferred the encapsulation process take place in a darkened (substantially light free) environment. As explained above, small molecules such as resveratrol lose their biological activity upon exposure to light and/or oxygen (due, e.g., to oxidation processes). Consequently, the composition containing small molecules should be mixed and/or encapsulated in a system including airtight and darkened mixing and filling chambers having a substantially oxygen-free environment. This can be achieved by using an enclosed system from which oxygen is removed. Oxygen may be removed using a vacuum, replacing the oxygen within the system with an inert gas flush, or a combination thereof. For example, the system can be purged of oxygen using a controlled nitrogen blanket. In addition, the system is kept substantially oxygen free through the use of a nitrogen flush during the encapsulation process. A nitrogen purge may also be used to remove oxygen from each individual capsule. Specifically, prior to sealing, a positive pressure can be applied to each capsule to replace any oxygen present within the capsule with nitrogen. Upon sealing, a nitrogen bubble remains within the capsule. A commercially available encapsulation system capable of filling capsules in a substantially oxygen-free and light-free environment is available from Capsugel (Greenwood, S.C.—www.capsugel.com), sold under the trade name CPS 1000 Capsule Filling Machine.

In a preferred embodiment, the compositions of the present embodiments are formulated as air-tight capsules in which encapsulation is conducted so as to prevent or minimize exposure to oxygen. In one embodiment, such encapsulation is conducted in an oxygen-free environment. For example, the components of the compositions of the present embodiments may be inserted into a capsule in an inert gas (e.g., nitrogen, argon, etc.) environment. Preferably, a nitrogen bubble (e.g., 5-20% of the capsule volume) may be introduced into the capsule to further stabilize and protect the components against oxidation (see, PCT Publication No. WO 01/08631, herein incorporated by reference). That international application has a corresponding U.S. patent application. Suitable capsules useful in the encapsulation of resveratrol and other oxidation prone ingredients of dietary supplements include Licaps® (Capsugel), an air-tight gelatin capsule. The presence of phytic acid, which has the ability to protect the components from metal-induced oxidation, augments such anti-oxidation precautions. A particularly preferred example of such a resveratrol-containing composition is Longevinex® (Resveratrol Partners, LLC, San Dimas, Calif.), which comprises resveratrol and phytic acid. Longevinex® contains as active ingredients (per capsule): 5 mg Vitamin E (as mixed tocopherols), 215 mg total resveratrol (obtained from French red wine and giant knotwood (Polygonum cuspidatum), and providing 100 mg of trans-resveratrol), 25 mg quercetin dihydrate, 75 mg phytic acid (rice bran extract), 380 mg rice bran oil, 55 mg sunflower lecithin.

Once a composition has been sealed into an air-tight capsule, it is important to maintain a low or no-oxygen environment in the packaging surrounding the capsules in order to protect the composition from oxidation should a break or leak occur in the sealed capsule. Therefore, an oxygen absorbing packette is preferably employed to reduce the presence of free oxygen. Vacuum or nitrogen-flushed packaging (bottles, pill cases, etc.) in air-tight materials is desirable.

In an alternative embodiment, the components and compositions of the present embodiments may be prepared as a microencapsulated process (see, generally, Rubiana, M. et al. (2004) Current Drug Targets, 5 (5):449-455). Micro-encapsulation is a process by which tiny particles or droplets (ranging in size from a few nanometers to one micron) are coated with a protective layer to create small capsules with controlled properties. Suitable micron-sized, encapsulated, preparations can be obtained using the microencapsulation processes of Maxx Performance Inc. (Chester, N.Y.), Blue California (Rancho Santa Margarita, Calif.), Southwest Research Institute (San Antonio, Tex.), Coating Place, Inc. (Verona, Wis.), Microtek Laboratories (Dayton, Ohio), Particle Sciences, Inc. (Bethlehem, Pa.), etc. 3^{rd}-generation Longevinex® (“Longevinex-3®”) (Resveratrol Partners, LLC), which contains Vitamin D3, Vitamin E, Resveratrol, Quercetin, and Phytic Acid is a particularly preferred microencapsulated form of the compositions of the present embodiments. The present embodiments further comprises a practical method of stabilizing quercetin and other easily oxidized dietary supplement ingredients which may come in contact with oxidizing metals.

Having now generally described the embodiments, the same will be more readily understood through reference to the following examples, which are provided by way of illustration and are not intended to be limiting of the present embodiments unless specified.

### Example 1

### Comparative Effects of Resveratrol and Compositions of the Present Embodiments

In order to determine if the compositions of the present embodiments were more effective than resveratrol alone in mediating a resveratrol biological activity, an analysis of gene expression was conducted, comparing the modulation of gene expression achieved by calorie restriction to the modulation of gene expression achieved by the compositions of the present embodiments.

Accordingly, the ability of resveratrol alone and the resveratrol-containing compositions of the present embodiments to up-regulate survival/longevity genes or down-regulate genes whose expression enhances cellular damage was compared using the expression profile of a calorie restricted (“CR”) animal as a positive control and the expression profile of a normally fed animal as a negative control. Male B6CHF1 mice (2 months of age) were thus either placed on a 40% calorie restricted diet, provided commercially obtained trans-resveratrol (Sigma Chemical; 1.25 mg/kg per day), provided a resveratrol-containing composition of the present embodiments (Longevinex®; Resveratrol Partners LLC; 100 mg trans-resveratrol containing capsule per 80 kg human per day (i.e., 2.5 mg/kg per day of resveratrol (1.25 mg/kg per day trans-resveratrol) 0.31 mg/kg per day quercetin dihydrate, 0.94 mg/kg per day rice bran extract, 4.75 mg/kg per day rice bran oil and 0.70 mg/kg per day sunflower lecithin)). The mice were monitored until they had reached five months of age.

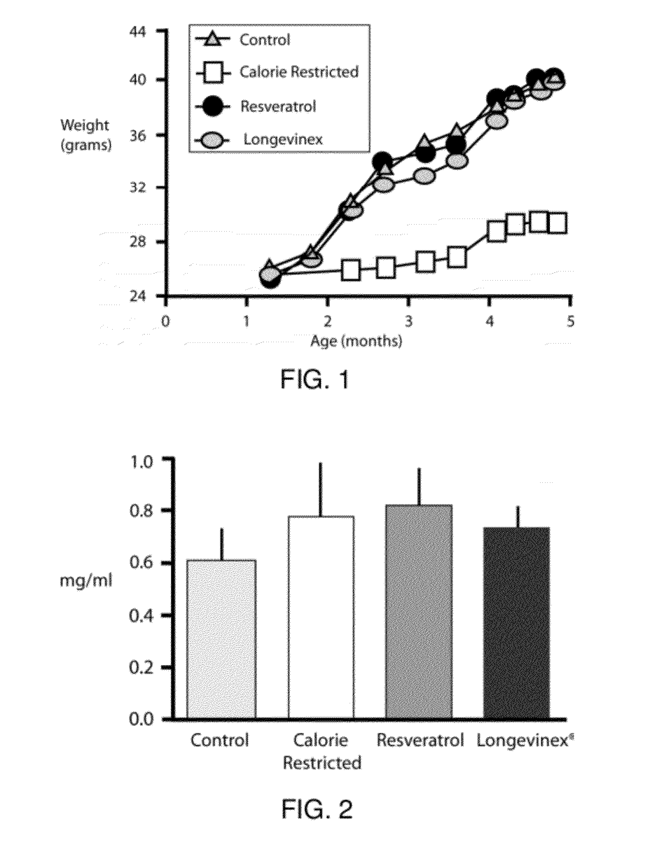

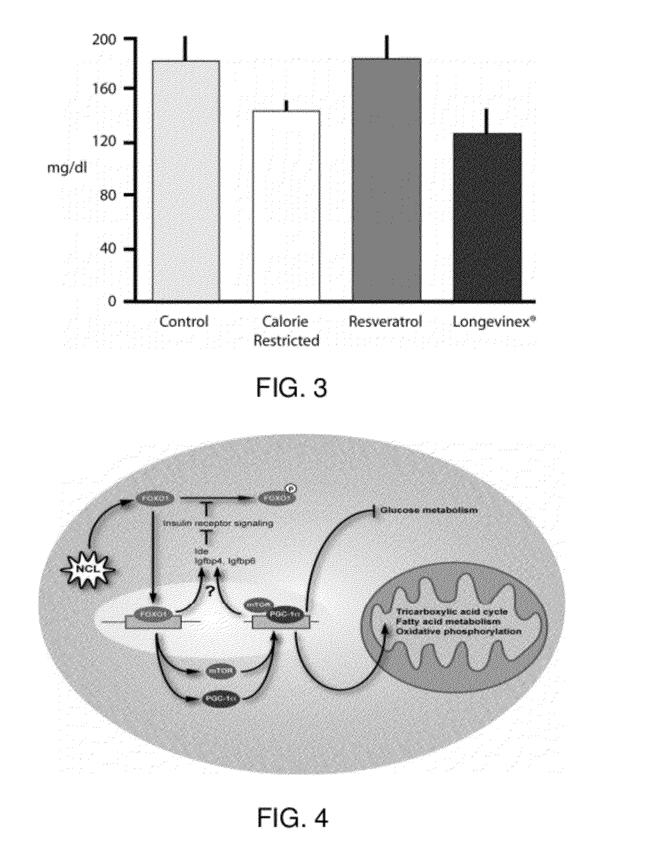

Body weight, serum glucose levels, serum insulin levels and lipid peroxidation in brain and muscle tissue were measured. The results showed that Longevinex® did not result in a weight increase distinguishable from control animals (FIG. 1). Serum insulin levels were found to be approximately the same as that observed in the calorie restricted animals (FIG. 2). Serum glucose levels were found to be lower than that observed in the calorie restricted animals (FIG. 3).

### Example 2

### Comparative Effects of Resveratrol and the Present Compositions on Gene Expression in Cardiac Tissue

The profile of expressed genes in the cardiac tissue of mice receiving resveratrol or a composition of the present embodiments (Longevinex®) was compared to that of mice placed on a calorie restricted diet and control mice. Gene expression was monitored using an Affymetrix MG430 2.0 Array, containing 45,101 probe sets per array. In cases in which the array represented the same gene with multiple probes, the probe set with the highest signal intensity was employed. Unknown genes (including uncharacterized ESTs and cDNA sequences were not analyzed. Thus, the array provided a means for analyzing 20,341 genes having a single Entrez Gene ID. Analysis was conducted substantially as described by Lee, C.-K. et al. (2002) Proc. Natl. Acad. Sci. (U.S.A.) 99:14988-14993, herein incorporated by reference. The mean of all arrays in a group were calculated. The means of treated groups were compared to the mean of the control group, and the statistical significance of any differences were determined using two-tailed t-tests (P<0.01). The results of the analysis are presented in Table 3 (submitted as a separate electronic file). In Table 3 the following abbreviations are used: CO, control; CR, calorie restricted; RES, resveratrol; LGX, Longevinex®; FC, fold change. FC is calculated as the mean of the treated group divided by the mean of the control group, and this value is then log-transformed (base 2) for statistical purposes. As an example, a gene that is expressed at 100 in the control and 200 in a treated group would be have an Fc of 2 (i.e., a twofold increase in expression); a gene that is expressed at 100 in the control and 50 in the treated group, would have an Fc of −2 (i.e., a twofold decrease in expression).

Treatment of human umbilical vein epithelial cells with ferulic acid, quercetin or resveratrol has been reported to result in changes to gene expression of greater than 2-fold down-regulation of 363 genes, and greater than 2-fold up-regulation of 233 genes of 10,000 genes probed (Nicholson, S. K. et al. (2008) Proc. Nutr. Soc. 67 (1):42-47). In contrast, Table 3 shows that 2,829 genes were found to exhibit a statistically significant change in expression in treated vs. control mice. Of these genes, 7% were found to exhibit altered expression in mice that had been subjected to only calorie restriction; 8% were found to exhibit altered expression in mice subjected only to resveratrol. Combining calorie restriction with resveratrol administration failed to alter the expression of any additional genes. In contrast, administration of Longevinex® was found to alter the expression of 61% of the 2,829 genes. Administration of Longevinex® to calorie restricted mice was found to alter the expression of an additional 2% of the genes. Administration of Longevinex® to mice receiving resveratrol was found to alter the expression of an additional 21% of the genes. Thus, Longevinex® alone or in combination with other regimens was found to affect 85% (2,406) of the total genes showing altered expression.

Several genes of particular interest showed expression patterns indicating that compositions of the present embodiments (Longevinex®) up-regulated survival/longevity genes or down-regulate genes whose expression enhances cellular damage to a greater extent than resveratrol, including the sirtuin family of genes, Pgc-1α, Uncoupling protein-3, and pyruvate dehydrogenase kinase 4.

The sirtuin family of genes, and in particular Sirtuin 1, are thought to be critical mediators of extended lifespans (Boily, G. et al. (2008) PLoS ONE 3 (3):e1759; Huang, J. et al. (2008) PLoS ONE 3 (3):e1710). Whereas mice receiving resveratrol showed only a 1.22 fold decrease in expression and mice subjected to a calorie restricted diet showed only a 1.12 fold reduction in Sirtuin 1 expression, expression of Sirtuin 1 was found to be decreased 1.71 fold in mice receiving Longevinex®. Pgc-1α (peroxisome proliferative activated receptor, gamma, coactivator 1 alpha; ppargc1a) is a transcriptional co-factor that controls energy metabolism and mitochondrial biogenesis; its expression is increased in skeletal muscle tissue upon long-term calorie restriction (Conley, K. E. et al. (2007) Curr. Opin. Clin. Nutr. Metab. Care. 10 (6):688-692; Wu, Z. et al. (2007) Expert Opin. Ther. Targets 11 (10):1329-1338). Whereas mice receiving resveratrol showed only a 1.6 fold increase in expression and mice subjected to a calorie restricted diet showed no increase in Pgc-1α expression, mice receiving Longevinex® showed a 1.94 fold increase in Pgc-1α expression.

Uncoupling protein-3 is believed to be a target of Pgc-1α and to play a role in fatty acid metabolism; its expression is increased in cardiac tissue upon long-term calorie restriction (Bézaire, V. et al. (Epub 2007 Jan. 3) FASEB J. 21 (2):312-324; Chan, C. B. et al. (2006) Curr. Diabetes Rev. 2 (3):271-283). Whereas mice receiving resveratrol showed only a 2.02 fold increase in expression and mice subjected to a calorie restricted diet showed only a 1.8 fold increase in uncoupling protein-3 expression, mice receiving Longevinex® showed a 2.79 fold increase in uncoupling protein-3 expression. Pyruvate dehydrogenase kinase 4 coordinates fuel selection during fasting to promote fatty acid metabolism (Sugden, M. C. et al. (2006) Arch. Physiol. Biochem. 112 (3):139-149; Pilegaard, H. et al. (2004) Proc. Nutr. Soc. 63 (2):221-226; Sugden, M. C. (2003) Obes. Res. 11 (2):167-169). It is a target of Pgc-1α and is induced in multiple tissues by long-term calorie restriction. Whereas mice receiving resveratrol showed only a 2.78 fold increase in expression and mice subjected to a calorie restricted diet showed only a 1.48 fold increase in pyruvate dehydrogenase kinase 4 expression, mice receiving Longevinex® showed a 3.25 fold increase in pyruvate dehydrogenase kinase 4 expression.

Analysis of the genes up-regulated or down-regulated by a compound of the present embodiments (Longevinex®) revealed that oxidative phosphorylation genes, which are involved in mitochondrial ATP production, were markedly up-regulated, as noted in Table 4.

TABLE 4

| FC CR | FC RES | FCLGX | Gene |
|---|---|---|---|
| 1.11 | 1.14 | 1.32 | Ndufa5 |
| −1.00 | −1.20 | −1.42 | Ndufaf1 |
| −1.04 | −1.13 | −1.22 | Ndufb3 |
| 1.13 | 1.06 | 1.27 | Ndufb8 |
| 1.12 | 1.18 | 1.28 | Ndufb7 |
| −1.34 | −1.55 | −2.65 | Ndufab1 |
| 1.07 | 1.20 | 1.51 | Ndufc1 |
| −1.07 | −1.30 | −1.39 | Ndufc2 |
| 1.08 | 1.11 | 1.37 | Ndufs1 |
| 1.13 | 1.10 | 1.26 | Ndufs2 |
| 1.09 | 1.12 | 1.23 | Ndufs3 |
| 1.04 | 1.19 | 1.33 | Ndufs5 |
| 1.13 | 1.18 | 1.44 | Ndufs7 |
| −1.02 | 1.03 | −1.23 | Ndufs8 |
| 1.14 | 1.18 | 1.21 | Ndufv1 |
| 1.11 | 1.13 | 1.34 | Ndufv2 |
| 1.17 | 1.13 | 1.43 | Sdha |
| 1.16 | 1.02 | 1.23 | Sdhd |
| 1.46 | 1.29 | 1.49 | Sulf2 |
| 1.01 | −1.25 | −1.33 | Uqcc |
| 1.10 | 1.19 | 1.34 | Uqcrc1 |
| 1.05 | 1.07 | 1.38 | Uqcrfs1 |
| 1.20 | 1.50 | 1.94 | Cox4i2 |
| 1.13 | 1.05 | 1.39 | Cox5a |
| 1.23 | 1.13 | 1.61 | Cox8a |

### Example 3

### Biochemical Pathways Affected by the Compositions of the Present Embodiments

Recent research has suggested that complex traits are emergent properties of molecular networks that are modulated by complex genetic loci and environmental factors. Chen, Y. et al. (Epub 2008 Mar. 16) Nature 452 (7186):429-435). Indeed, research within the last decade has revealed that most chronic illnesses such as cancer, cardiovascular and pulmonary diseases, neurological diseases, diabetes, and autoimmune diseases exhibit dysregulation of multiple cell signaling pathways (Harikumar, K. B. et al. (Epub Feb. 15, 2008) Cell Cycle. 2008:7 (8)). The compounds of the present embodiments were therefore evaluated for their effect on the expression of biochemical pathways and were found to affect the expression of genes involved in 220 biological processes (P<0.05), as shown in Table 5.

TABLE 5

| | | | Changed | Genes | | | |
|---|---|---|---|---|---|---|---|
| GO ID | Biological Processes | Treatment | by LT-CR | in Series | CR | RES | LGX |
| GO: 0051128 | Regulation Of Cellular Component | CR only | 0.0277 | 51 | 5 | | |
| | Organization And Biogenesis | | | | | | |
| GO: 0001558 | Regulation Of Cell Growth | CR only | | 74 | 5 | | |
| GO: 0006820 | Anion Transport | CR only | | 155 | 6 | | |
| GO: 0008361 | Regulation Of Cell Size | CR only | | 102 | 6 | | |
| GO: 0016049 | Cell Growth | CR only | | 90 | 5 | | |
| GO: 0030217 | T Cell Differentiation | CR only | | 55 | 3 | | |
| GO: 0030595 | Leukocyte Chemotaxis | CR only | | 18 | 2 | | |
| GO: 0045580 | Regulation Of T Cell Differentiation | CR only | | 15 | 2 | | |
| GO: 0045792 | Negative Regulation Of Cell Size | CR only | | 16 | 2 | | |
| GO: 0048705 | Skeletal Morphogenesis | CR only | | 20 | 2 | | |
| GO: 0051246 | Regulation Of Protein Metabolic Process | CR only | | 204 | 8 | | |
| GO: 0033554 | Cellular Response To Stress | RES only | 0.0074 | 14 | | 3 | |
| GO: 0006888 | ER To Golgi Vesicle-Mediated Transport | RES only | 0.0284 | 16 | | 3 | |
| GO: 0000723 | Telomere Maintenance | RES only | | 17 | | 3 | |
| GO: 0001958 | Endochondral Ossification | RES only | | 8 | | 2 | |
| GO: 0006281 | DNA Repair | RES only | | 178 | | 13 | |
| GO: 0006353 | Transcription Termination | RES only | | 6 | | 2 | |
| GO: 0006446 | Regulation Of Translational Initiation | RES only | | 20 | | 4 | |
| GO: 0006596 | Polyamine Biosynthetic Process | RES only | | 5 | | 2 | |
| GO: 0006625 | Protein Targeting To Peroxisome | RES only | | 5 | | 2 | |
| GO: 0006825 | Copper Ion Transport | RES only | | 9 | | 3 | |
| GO: 0006919 | Caspase Activation | RES only | | 16 | | 3 | |
| GO: 0006974 | Response To DNA Damage Stimulus | RES only | | 217 | | 15 | |
| GO: 0006983 | ER Overload Response | RES only | | 5 | | 2 | |
| GO: 0007017 | Microtubule-Based Process | RES only | | 155 | | 12 | |
| GO: 0007091 | Mitotic Metaphase/Anaphase Transition | RES only | | 8 | | 2 | |
| GO: 0007143 | Female Meiosis | RES only | | 8 | | 2 | |
| GO: 0008299 | Isoprenoid Biosynthetic Process | RES only | | 19 | | 3 | |
| GO: 0045351 | Interferon Type I Biosynthetic Process | RES only | | 6 | | 2 | |
| GO: 0045577 | Regulation Of B Cell Differentiation | RES only | | 8 | | 2 | |
| GO: 0046330 | Positive Regulation Of JNK Cascade | RES only | | 8 | | 2 | |
| GO: 0048193 | Golgi Vesicle Transport | RES only | | 37 | | 6 | |
| GO: 0050673 | Epithelial Cell Proliferation | RES only | | 30 | | 4 | |
| GO: 0006119 | Oxidative Phosphorylation | LGX only | 0.0001 | 39 | | | 10 |
| GO: 0042773 | ATP Synthesis Coupled Electron | LGX only | 0.0019 | 11 | | | 5 |
| | Transport | | | | | | |
| GO: 0030036 | Actin Cytoskeleton Organization And | LGX only | 0.0024 | 146 | | | 34 |
| | Biogenesis | | | | | | |
| GO: 0006629 | Lipid Metabolic Process | LGX only | 0.0146 | 535 | | | 89 |
| GO: 0044255 | Cellular Lipid Metabolic Process | LGX only | 0.0147 | 459 | | | 80 |
| GO: 0001701 | In Utero Embryonic Development | LGX only | 0.0195 | 101 | | | 19 |
| GO: 0040008 | Regulation Of Growth | LGX only | 0.0242 | 135 | | | 23 |
| GO: 0000375 | RNA Splicing, Via Transesterification | LGX only | 0.0251 | 39 | | | 10 |
| | Reactions | | | | | | |
| GO: 0000398 | Nuclear Mrna Splicing, Via Spliceosome | LGX only | 0.0251 | 39 | | | 10 |
| GO: 0006366 | Transcription From RNA Polymerase II | LGX only | 0.0264 | 392 | | | 72 |
| | Promoter | | | | | | |
| GO: 0006357 | Regulation Of Transcription From RNA | LGX only | 0.0276 | 351 | | | 59 |
| | Polymerase II Promoter | | | | | | |
| GO: 0016044 | Membrane Organization And Biogenesis | LGX only | 0.0292 | 209 | | | 34 |
| GO: 0006066 | Alcohol Metabolic Process | LGX only | 0.0299 | 222 | | | 41 |
| GO: 0065002 | Intracellular Protein Transport Across A | LGX only | 0.0372 | 59 | | | 16 |
| | Membrane | | | | | | |
| GO: 0006099 | Tricarboxylic Acid Cycle | LGX only | 0.0396 | 23 | | | 7 |
| GO: 0009060 | Aerobic Respiration | LGX only | 0.0396 | 24 | | | 7 |
| GO: 0044265 | Cellular Macromolecule Catabolic | LGX only | 0.0417 | 201 | | | 41 |
| | Process | | | | | | |
| GO: 0006006 | Glucose Metabolic Process | LGX only | 0.0441 | 83 | | | 16 |
| GO: 0045333 | Cellular Respiration | LGX only | 0.0484 | 28 | | | 8 |
| GO: 0000038 | Very-Long-Chain Fatty Acid Metabolic | LGX only | | 5 | | | 3 |
| | Process | | | | | | |
| GO: 0000059 | Protein Import Into Nucleus, Docking | LGX only | | 15 | | | 7 |
| GO: 0000186 | Activation Of MAPKK Activity | LGX only | | 10 | | | 5 |
| GO: 0001525 | Angiogenesis | LGX only | | 124 | | | 26 |
| GO: 0001568 | Blood Vessel Development | LGX only | | 188 | | | 42 |
| GO: 0001570 | Vasculogenesis | LGX only | | 27 | | | 8 |
| GO: 0001839 | Neural Plate Morphogenesis | LGX only | | 40 | | | 9 |
| GO: 0001841 | Neural Tube Formation | LGX only | | 39 | | | 9 |
| GO: 0001843 | Neural Tube Closure | LGX only | | 29 | | | 7 |
| GO: 0001935 | Endothelial Cell Proliferation | LGX only | | 8 | | | 4 |
| GO: 0002026 | Cardiac Inotropy | LGX only | | 10 | | | 4 |
| GO: 0003007 | Heart Morphogenesis | LGX only | | 32 | | | 8 |
| GO: 0005978 | Glycogen Biosynthetic Process | LGX only | | 11 | | | 5 |
| GO: 0006007 | Glucose Catabolic Process | LGX only | | 44 | | | 11 |
| GO: 0006098 | Pentose-Phosphate Shunt | LGX only | | 7 | | | 3 |
| GO: 0006118 | Electron Transport | LGX only | | 303 | | | 65 |
| GO: 0006120 | Mitochondrial Electron Transport, | LGX only | | 6 | | | 5 |
| | NADH To Ubiquinone | | | | | | |
| GO: 0006171 | Camp Biosynthetic Process | LGX only | | 14 | | | 5 |
| GO: 0006259 | DNA Metabolic Process | LGX only | | 524 | | | 77 |
| GO: 0006323 | DNA Packaging | LGX only | | 209 | | | 37 |
| GO: 0006325 | Establishment And/Or Maintenance Of | LGX only | | 203 | | | 34 |
| | Chromatin Architecture | | | | | | |
| GO: 0006333 | Chromatin Assembly Or Disassembly | LGX only | | 80 | | | 15 |
| GO: 0006352 | Transcription Initiation | LGX only | | 27 | | | 7 |
| GO: 0006354 | RNA Elongation | LGX only | | 5 | | | 3 |
| GO: 0006367 | Transcription Initiation From RNA | LGX only | | 11 | | | 5 |
| | Polymerase II Promoter | | | | | | |
| GO: 0006396 | RNA Processing | LGX only | | 319 | | | 63 |
| GO: 0006397 | Mrna Processing | LGX only | | 213 | | | 43 |
| GO: 0006414 | Translational Elongation | LGX only | | 19 | | | 6 |
| GO: 0006461 | Protein Complex Assembly | LGX only | | 122 | | | 28 |
| GO: 0006468 | Protein Amino Acid Phosphorylation | LGX only | | 545 | | | 83 |
| GO: 0006470 | Protein Amino Acid Dephosphorylation | LGX only | | 99 | | | 18 |
| GO: 0006473 | Protein Amino Acid Acetylation | LGX only | | 12 | | | 4 |
| GO: 0006508 | Proteolysis | LGX only | | 545 | | | 85 |
| GO: 0006520 | Amino Acid Metabolic Process | LGX only | | 198 | | | 34 |
| GO: 0006606 | Protein Import Into Nucleus | LGX only | | 53 | | | 13 |
| GO: 0006612 | Protein Targeting To Membrane | LGX only | | 15 | | | 5 |
| GO: 0006631 | Fatty Acid Metabolic Process | LGX only | | 142 | | | 35 |
| GO: 0006635 | Fatty Acid Beta-Oxidation | LGX only | | 13 | | | 6 |
| GO: 0006638 | Neutral Lipid Metabolic Process | LGX only | | 21 | | | 6 |
| GO: 0006641 | Triacylglycerol Metabolic Process | LGX only | | 17 | | | 6 |
| GO: 0006662 | Glycerol Ether Metabolic Process | LGX only | | 23 | | | 6 |
| GO: 0006766 | Vitamin Metabolic Process | LGX only | | 57 | | | 14 |
| GO: 0006807 | Nitrogen Compound Metabolic Process | LGX only | | 315 | | | 49 |
| GO: 0006869 | Lipid Transport | LGX only | | 67 | | | 16 |
| GO: 0006913 | Nucleocytoplasmic Transport | LGX only | | 89 | | | 23 |
| GO: 0006914 | Autophagy | LGX only | | 21 | | | 8 |
| GO: 0007031 | Peroxisome Organization And | LGX only | | 22 | | | 7 |
| | Biogenesis | | | | | | |
| GO: 0007182 | Common-Partner SMAD Protein | LGX only | | 8 | | | 4 |
| | Phosphorylation | | | | | | |
| GO: 0007190 | Adenylate Cyclase Activation | LGX only | | 12 | | | 4 |
| GO: 0007242 | Intracellular Signaling Cascade | LGX only | | 915 | | | 133 |
| GO: 0007369 | Gastrulation | LGX only | | 55 | | | 13 |
| GO: 0007498 | Mesoderm Development | LGX only | | 44 | | | 13 |
| GO: 0007507 | Heart Development | LGX only | | 158 | | | 39 |
| GO: 0007512 | Adult Heart Development | LGX only | | 9 | | | 4 |
| GO: 0007517 | Muscle Development | LGX only | | 105 | | | 19 |
| GO: 0008016 | Regulation Of Heart Contraction | LGX only | | 27 | | | 9 |
| GO: 0008286 | Insulin Receptor Signaling Pathway | LGX only | | 24 | | | 7 |
| GO: 0009308 | Amine Metabolic Process | LGX only | | 294 | | | 46 |
| GO: 0009653 | Anatomical Structure Morphogenesis | LGX only | | 993 | | | 143 |
| GO: 0009790 | Embryonic Development | LGX only | | 387 | | | 61 |
| GO: 0009792 | Embryonic Development Ending In Birth | LGX only | | 190 | | | 32 |
| | Or Egg Hatching | | | | | | |
| GO: 0010003 | Gastrulation (Sensu Mammalia) | LGX only | | 17 | | | 6 |
| GO: 0015804 | Neutral Amino Acid Transport | LGX only | | 6 | | | 3 |
| GO: 0015908 | Fatty Acid Transport | LGX only | | 6 | | | 3 |
| GO: 0016071 | Mrna Metabolic Process | LGX only | | 240 | | | 45 |
| GO: 0016192 | Vesicle-Mediated Transport | LGX only | | 365 | | | 61 |
| GO: 0016310 | Phosphorylation | LGX only | | 601 | | | 95 |
| GO: 0016311 | Dephosphorylation | LGX only | | 111 | | | 20 |
| GO: 0016481 | Negative Regulation Of Transcription | LGX only | | 223 | | | 40 |
| GO: 0016485 | Protein Processing | LGX only | | 58 | | | 14 |
| GO: 0016540 | Protein Autoprocessing | LGX only | | 30 | | | 9 |
| GO: 0016567 | Protein Ubiquitination | LGX only | | 36 | | | 9 |
| GO: 0016568 | Chromatin Modification | LGX only | | 152 | | | 27 |
| GO: 0016574 | Histone Ubiquitination | LGX only | | 5 | | | 3 |
| GO: 0019395 | Fatty Acid Oxidation | LGX only | | 20 | | | 8 |
| GO: 0019752 | Carboxylic Acid Metabolic Process | LGX only | | 403 | | | 78 |
| GO: 0030163 | Protein Catabolic Process | LGX only | | 162 | | | 31 |
| GO: 0030239 | Myofibril Assembly | LGX only | | 12 | | | 5 |
| GO: 0030323 | Respiratory Tube Development | LGX only | | 58 | | | 12 |
| GO: 0030324 | Lung Development | LGX only | | 57 | | | 12 |
| GO: 0030855 | Epithelial Cell Differentiation | LGX only | | 34 | | | 8 |
| GO: 0030856 | Regulation Of Epithelial Cell | LGX only | | 7 | | | 3 |
| | Differentiation | | | | | | |
| GO: 0030865 | Cortical Cytoskeleton Organization And | LGX only | | 10 | | | 5 |
| | Biogenesis | | | | | | |
| GO: 0031032 | Actomyosin Structure Organization And | LGX only | | 16 | | | 5 |
| | Biogenesis | | | | | | |
| GO: 0032147 | Activation Of Protein Kinase Activity | LGX only | | 28 | | | 8 |
| GO: 0035051 | Cardiac Cell Differentiation | LGX only | | 13 | | | 6 |
| GO: 0035239 | Tube Morphogenesis | LGX only | | 128 | | | 25 |
| GO: 0035295 | Tube Development | LGX only | | 174 | | | 36 |
| GO: 0042254 | Ribosome Biogenesis And Assembly | LGX only | | 97 | | | 18 |
| GO: 0042692 | Muscle Cell Differentiation | LGX only | | 58 | | | 14 |
| GO: 0043009 | Chordate Embryonic Development | LGX only | | 187 | | | 32 |
| GO: 0043087 | Regulation Of Gtpase Activity | LGX only | | 59 | | | 12 |
| GO: 0043623 | Cellular Protein Complex Assembly | LGX only | | 39 | | | 11 |
| GO: 0043631 | RNA Polyadenylation | LGX only | | 11 | | | 4 |
| GO: 0044257 | Cellular Protein Catabolic Process | LGX only | | 116 | | | 27 |
| GO: 0045214 | Sarcomere Organization | LGX only | | 9 | | | 4 |
| GO: 0045761 | Regulation Of Adenylate Cyclase | LGX only | | 16 | | | 5 |
| | Activity | | | | | | |
| GO: 0045893 | Positive Regulation Of Transcription, | LGX only | | 225 | | | 44 |
| | DNA-Dependent | | | | | | |
| GO: 0045944 | Positive Regulation Of Transcription | LGX only | | 186 | | | 34 |
| | From RNA Polymerase II Promoter | | | | | | |
| GO: 0046058 | Camp Metabolic Process | LGX only | | 17 | | | 5 |
| GO: 0046777 | Protein Amino Acid | LGX only | | 29 | | | 9 |
| | Autophosphorylation | | | | | | |
| GO: 0048276 | Gastrulation (Sensu Vertebrata) | LGX only | | 24 | | | 7 |
| GO: 0048514 | Blood Vessel Morphogenesis | LGX only | | 160 | | | 36 |
| GO: 0048646 | Anatomical Structure Formation | LGX only | | 171 | | | 34 |
| GO: 0050658 | RNA Transport | LGX only | | 48 | | | 11 |
| GO: 0051028 | Mrna Transport | LGX only | | 45 | | | 11 |
| GO: 0051146 | Striated Muscle Cell Differentiation | LGX only | | 26 | | | 11 |
| GO: 0051170 | Nuclear Import | LGX only | | 54 | | | 13 |
| GO: 0055001 | Muscle Cell Development | LGX only | | 13 | | | 5 |
| GO: 0055002 | Striated Muscle Cell Development | LGX only | | 12 | | | 5 |
| GO: 0055007 | Cardiac Muscle Cell Differentiation | LGX only | | 9 | | | 6 |
| GO: 0055012 | Ventricular Cardiac Muscle Cell | LGX only | | 6 | | | 4 |
| | Differentiation | | | | | | |
| GO: 0051016 | Barbed-End Actin Filament Capping | CR & RES | 0.0487 | 17 | 2 | 3 | |
| GO: 0030029 | Actin Filament-Based Process | CR & LGX | 0.0049 | 157 | 6 | | 37 |
| GO: 0006084 | Acetyl-Coa Metabolic Process | CR & LGX | 0.0068 | 33 | 3 | | 11 |
| GO: 0045941 | Positive Regulation Of Transcription | CR & LGX | 0.0119 | 267 | 8 | | 48 |
| GO: 0006915 | Apoptosis | CR & LGX | 0.0172 | 537 | 14 | | 84 |
| GO: 0012501 | Programmed Cell Death | CR & LGX | 0.0198 | 544 | 14 | | 84 |
| GO: 0046356 | Acetyl-Coa Catabolic Process | CR & LGX | 0.0396 | 24 | 2 | | 8 |
| GO: 0008219 | Cell Death | CR & LGX | 0.0410 | 564 | 14 | | 84 |
| GO: 0006364 | Rrna Processing | CR & LGX | | 50 | 3 | | 11 |
| GO: 0006519 | Amino Acid And Derivative Metabolic | CR & LGX | | 253 | 8 | | 41 |
| | Process | | | | | | |
| GO: 0006796 | Phosphate Metabolic Process | CR & LGX | | 714 | 17 | | 114 |
| GO: 0006839 | Mitochondrial Transport | CR & LGX | | 20 | 2 | | 9 |
| GO: 0007005 | Mitochondrion Organization And | CR & LGX | | 58 | 4 | | 19 |
| | Biogenesis | | | | | | |
| GO: 0007167 | Enzyme Linked Receptor Protein | CR & LGX | | 252 | 9 | | 46 |
| | Signaling Pathway | | | | | | |
| GO: 0007179 | Transforming Growth Factor Beta | CR & LGX | | 42 | 3 | | 11 |
| | Receptor Signaling Pathway | | | | | | |
| GO: 0009056 | Catabolic Process | CR & LGX | | 474 | 13 | | 79 |
| GO: 0016265 | Death | CR & LGX | | 564 | 14 | | 84 |
| GO: 0030833 | Regulation Of Actin Filament | CR & LGX | | 10 | 2 | | 4 |
| | Polymerization | | | | | | |
| GO: 0008104 | Protein Localization | RES & LGX | 0.0007 | 663 | | 38 | 127 |
| GO: 0015031 | Protein Transport | RES & LGX | 0.0011 | 581 | | 38 | 121 |
| GO: 0045184 | Establishment Of Protein Localization | RES & LGX | 0.0028 | 610 | | 38 | 123 |
| GO: 0006886 | Intracellular Protein Transport | RES & LGX | 0.0098 | 357 | | 26 | 75 |
| GO: 0006605 | Protein Targeting | RES & LGX | 0.0099 | 161 | | 12 | 34 |
| GO: 0044249 | Cellular Biosynthetic Process | RES & LGX | 0.0107 | 710 | | 45 | 117 |
| GO: 0009058 | Biosynthetic Process | RES & LGX | 0.0120 | 979 | | 60 | 155 |
| GO: 0006412 | Translation | RES & LGX | 0.0364 | 338 | | 25 | 62 |
| GO: 0044262 | Cellular Carbohydrate Metabolic Process | RES & LGX | 0.0471 | 221 | | 18 | 49 |
| GO: 0005975 | Carbohydrate Metabolic Process | RES & LGX | | 316 | | 21 | 60 |
| GO: 0005976 | Polysaccharide Metabolic Process | RES & LGX | | 42 | | 9 | 15 |
| GO: 0005977 | Glycogen Metabolic Process | RES & LGX | | 31 | | 7 | 13 |
| GO: 0006091 | Generation Of Precursor Metabolites | RES & LGX | | 390 | | 24 | 88 |
| | And Energy | | | | | | |
| GO: 0006112 | Energy Reserve Metabolic Process | RES & LGX | | 35 | | 7 | 13 |
| GO: 0006413 | Translational Initiation | RES & LGX | | 40 | | 6 | 9 |
| GO: 0006511 | Ubiquitin-Dependent Protein Catabolic | RES & LGX | | 109 | | 9 | 27 |
| | Process | | | | | | |
| GO: 0006512 | Ubiquitin Cycle | RES & LGX | | 356 | | 27 | 79 |
| GO: 0007178 | Transmembrane Receptor Protein | RES & LGX | | 75 | | 7 | 19 |
| | Serine/Threonine Kinase Signaling | | | | | | |
| | Pathway | | | | | | |
| GO: 0007264 | Small Gtpase Mediated Signal | RES & LGX | | 320 | | 22 | 62 |
| | Transduction | | | | | | |
| GO: 0008380 | RNA Splicing | RES & LGX | | 162 | | 12 | 30 |
| GO: 0009059 | Macromolecule Biosynthetic Process | RES & LGX | | 525 | | 35 | 90 |
| GO: 0019941 | Modification-Dependent Protein | RES & LGX | | 111 | | 9 | 27 |
| | Catabolic Process | | | | | | |
| GO: 0043085 | Positive Regulation Of Enzyme Activity | RES & LGX | | 40 | | 5 | 11 |
| GO: 0043280 | Positive Regulation Of Caspase Activity | RES & LGX | | 17 | | 3 | 5 |
| GO: 0043281 | Regulation Of Caspase Activity | RES & LGX | | 27 | | 5 | 8 |
| GO: 0045454 | Cell Redox Homeostasis | RES & LGX | | 40 | | 6 | 9 |
| GO: 0050790 | Regulation Of Catalytic Activity | RES & LGX | | 241 | | 21 | 53 |
| GO: 0008064 | Regulation Of Actin Polymerization | All | 0.0139 | 30 | 5 | 4 | 9 |
| | And/Or Depolymerization | | | | | | |
| GO: 0030832 | Regulation Of Actin Filament Length | All | 0.0139 | 31 | 5 | 4 | 9 |
| GO: 0046907 | Intracellular Transport | All | 0.0287 | 523 | 16 | 43 | 113 |
| GO: 0008154 | Actin Polymerization And/Or | All | 0.0414 | 39 | 5 | 6 | 12 |
| | Depolymerization | | | | | | |
| GO: 0051649 | Establishment Of Cellular Localization | All | 0.0467 | 653 | 17 | 45 | 125 |
| GO: 0006457 | Protein Folding | All | | 124 | 6 | 13 | 34 |
| GO: 0006996 | Organelle Organization And Biogenesis | All | | 897 | 27 | 53 | 158 |
| GO: 0007010 | Cytoskeleton Organization And | All | | 403 | 12 | 26 | 70 |
| | Biogenesis | | | | | | |
| GO: 0007018 | Microtubule-Based Movement | All | | 72 | 4 | 9 | 14 |
| GO: 0030041 | Actin Filament Polymerization | All | | 17 | 2 | 3 | 7 |
| GO: 0051258 | Protein Polymerization | All | | 32 | 6 | 6 | 8 |

Calorie restriction affected genes associated with 5% of these processes, administration of resveratrol affected genes associated with 10% of these processes. Compounds of the present embodiments (e.g., Longevinex®) were found to affect 85% of these processes. Administration of resveratrol to calorie restricted mice failed to affect any genes in any of these processes. Administration of Longevinex® to calorie restricted mice was found to affect genes associated with 8% of these processes. Administration of both resveratrol and Longevinex® was found to affect genes associated with 12% of these processes. Table 6 shows the modulation of the genes of the oxidative phosphorylation pathway (GO:0006119) caused by calorie restriction (CR), resveratrol alone (Res), or Longevinex® (LGX).

TABLE 6
Modulation Of The Genes Of The Oxidative
Phosphorylation Pathway (GO: 0006119)

| | Fold Change | | |
|---|---|---|---|
| Gene | CR | Res | LGX |
| 1110020P15Rik | 1.06 | 1.09 | 1.26 |
| Atp5a1 | 1.09 | 1.02 | 1.29 |
| Atp5b | 1.10 | 1.01 | 1.27 |
| Atp5c1 | −1.16 | −1.28 | −1.16 |
| Atp5f1 | 1.09 | 1.08 | 1.19 |
| Atp5g1 | 1.26 | −1.58 | −1.17 |
| Atp5g3 | 1.06 | −1.11 | −1.01 |
| Atp5h | 1.07 | 1.04 | 1.37 |
| Atp5j | 1.07 | −1.00 | 1.24 |
| Atp5k | 1.05 | 1.02 | 1.24 |
| Atp6v0d1 | 1.03 | −1.21 | −1.19 |
| Atp6v0d2 | 1.04 | 1.65 | 1.21 |
| Atp6v1a | −1.04 | −1.18 | −1.35 |
| Atp6v1b2 | 1.27 | −1.16 | −1.23 |
| Atp6v1c1 | −1.10 | −1.07 | −1.06 |
| Atp6v1c2 | 2.17 | 1.42 | −1.00 |
| Atp6v1d | 1.13 | −1.09 | −1.05 |
| Atp6v1e1 | 1.05 | −1.42 | −1.42 |
| Atp6v1e2 | 1.11 | 1.28 | 1.30 |
| Atp6v1f | −1.14 | −1.21 | −1.44 |
| Atp6v1h | −1.17 | −1.19 | −1.11 |
| Atp7a | −1.13 | −1.18 | −1.29 |
| Cyc1 | 1.14 | 1.08 | 1.29 |
| Msh2 | 1.05 | −1.03 | −1.15 |
| Ndufa7 | −1.07 | −1.14 | −1.07 |
| Ndufb9 | 1.07 | 1.07 | 1.25 |
| Ndufc2 | −1.07 | −1.30 | −1.39 |
| Ndufs1 | 1.08 | 1.11 | 1.37 |
| Ndufs3 | 1.09 | 1.12 | 1.23 |
| Ndufs7 | 1.13 | 1.18 | 1.44 |
| Ndufv1 | 1.14 | 1.18 | 1.21 |
| Uqcr | 1.03 | 1.10 | 1.30 |
| Uqcrb | −1.09 | −1.00 | −1.22 |
| Uqcrh | 1.05 | −1.40 | −1.34 |

Table 7 shows the modulation of the genes of the glucose metabolism pathway (GO:0006006) caused by calorie restriction (CR), resveratrol alone (Res), or the compositions of the present embodiments (LGX).

TABLE 7
Modulation Of The Genes Of The Glucose
Metabolism Pathway (GO: 0006006)

| | Fold Change | | |
|---|---|---|---|
| Gene | CR | Res | LGX |
| 6430537H07Rik | −1.26 | −1.73 | −1.04 |
| Acn9 | −1.04 | −1.08 | −1.22 |
| Adipoq | 1.74 | 1.24 | 2.14 |
| Adpgk | −1.02 | 1.24 | 1.37 |
| Akt1 | 1.11 | 1.66 | 1.73 |
| Aldoart1 | 2.17 | 2.37 | 3.08 |
| Aldoart2 | −1.05 | 1.16 | 1.05 |
| Aldob | −1.06 | 1.24 | 1.21 |
| Aldoc | −1.32 | 1.14 | 1.32 |
| Atf3 | −1.86 | −1.47 | −1.68 |
| Atf4 | −1.06 | 1.07 | 1.10 |
| Bad | 1.05 | 1.10 | −1.07 |
| Bpgm | −1.28 | 1.35 | 1.57 |
| Cacna1a | −1.31 | 1.03 | −1.21 |
| Car5a | −2.59 | 1.19 | −1.14 |
| Dcxr | 1.05 | 1.08 | 1.14 |
| Dhtkd1 | −1.45 | −1.32 | −1.08 |
| Dlat | 1.05 | −1.09 | −1.07 |
| Eno2 | −1.19 | 1.32 | 1.86 |
| Eno3 | 1.17 | 1.26 | 1.37 |
| Fabp5 | 1.14 | −1.13 | −1.26 |
| Fbp1 | −1.50 | −1.25 | 1.20 |
| Fbp2 | −1.16 | 1.42 | 1.47 |
| G6pc | 1.42 | −1.62 | −1.71 |
| G6pd2 | 1.93 | 1.37 | 2.90 |
| G6pdx | 1.30 | 1.00 | 1.38 |
| Ganc | −1.26 | 1.08 | −1.30 |
| Gapdh | 1.15 | 1.09 | 1.35 |
| Gapdhs | 1.35 | 1.62 | 1.50 |
| Gck | 1.16 | 1.26 | 1.16 |
| Gpd1 | 1.83 | 1.51 | 2.41 |
| Gpd2 | 1.48 | −1.41 | 1.17 |
| H6pd | 1.33 | 1.43 | 2.06 |
| Hibadh | 1.09 | 1.17 | 1.15 |
| Hk1 | 1.12 | −1.24 | −1.34 |
| Hk3 | 2.23 | 1.60 | 1.77 |
| Hkdc1 | 2.64 | 2.10 | 1.68 |
| Ins1 | 1.19 | 1.49 | 1.37 |
| Ldha | 1.10 | −1.03 | 1.04 |
| Ldhal6b | 1.15 | 1.45 | 2.73 |
| Ldhb | 1.21 | 1.35 | 1.60 |
| Ldhc | 1.94 | 2.25 | 2.44 |
| Lep | −1.57 | −1.96 | −1.43 |
| Lrrc16 | 1.00 | −1.10 | 1.03 |
| Mapk14 | −1.10 | −1.13 | −1.51 |
| Mdh1 | 1.07 | 1.08 | 1.41 |
| Mdh2 | 1.10 | 1.01 | 1.15 |
| Npy1r | 1.11 | 1.37 | 1.53 |
| Nr3c1 | 1.14 | −1.14 | 1.10 |
| Ogdh | 1.32 | 1.23 | 1.38 |
| Onecut1 | −1.32 | −2.13 | −2.05 |
| Pck1 | 1.68 | 2.36 | 4.03 |
| Pck2 | 1.06 | −1.09 | −1.05 |
| Pcx | 1.40 | 1.33 | 1.60 |
| Pdha1 | 1.12 | 1.09 | 1.30 |
| Pdha2 | 1.78 | −1.03 | 1.57 |
| Pdk1 | −1.01 | 1.01 | −1.18 |
| Pdk2 | 1.11 | 1.11 | 1.24 |
| Pdk3 | −2.02 | −1.04 | 1.21 |
| Pdk4 | 1.48 | 2.78 | 3.25 |
| Pdx1 | −3.03 | −1.37 | −1.14 |
| Pfkl | 1.19 | −1.15 | −1.11 |
| Pfkm | 1.13 | 1.07 | 1.02 |
| Pfkp | 1.32 | 1.13 | 1.24 |
| Pgam1 | 1.15 | 1.01 | 1.07 |
| Pgam2 | 1.27 | 1.38 | 1.81 |
| Pgd | 1.20 | −1.05 | 1.16 |
| Pgk2 | −1.79 | 1.29 | −1.01 |
| Pgls | 1.28 | 1.24 | 1.42 |
| Pgm1 | 1.05 | 1.14 | 1.21 |
| Pgm2 | 1.11 | 1.09 | 1.03 |
| Pgm2l1 | 1.19 | 1.31 | 1.11 |
| Pgm3 | 1.13 | −1.09 | −1.29 |
| Pik3ca | −1.05 | 1.34 | 1.21 |
| Pklr | 1.12 | −1.08 | −1.47 |
| Pkm2 | 1.31 | 1.34 | 1.70 |
| Ppara | 1.14 | −1.06 | −1.28 |
| Prkaa1 | −1.08 | 1.17 | −1.38 |
| Rpia | 1.04 | 1.06 | 1.12 |
| Sds | −1.65 | −1.16 | 2.01 |
| Slc2a8 | 1.11 | 1.30 | 1.19 |
| Taldo1 | 1.12 | 1.04 | 1.08 |
| Tnf | −1.16 | −1.73 | 1.11 |
| Tpi1 | 1.15 | 1.06 | 1.24 |
| Uevld | −1.01 | −1.12 | −1.28 |

Table 8 shows the modulation of the genes of the tricarboxylic acid metabolism pathway (GO:0006099) caused by calorie restriction (CR), resveratrol alone (Res), or the compositions of the present embodiments (LGX).

TABLE 8
Modulation Of The Genes Of The Tricarboxylic
Acid Metabolism Pathway (GO: 0006099)

| | Fold Change | | |
|---|---|---|---|
| Gene | CR | Res | LGX |
| 2610507B11Rik | 1.10 | 1.06 | 1.25 |
| Aco1 | 1.00 | −1.01 | −1.05 |
| Aco2 | 1.16 | 1.13 | 1.39 |
| Atp5g3 | 1.06 | −1.11 | −1.01 |
| Cs | 1.22 | 1.07 | 1.46 |
| Dlst | 1.21 | 1.08 | 1.24 |
| Fh1 | −1.01 | 1.05 | 1.18 |
| Idh2 | 1.12 | 1.21 | 1.36 |
| Idh3a | 1.16 | −1.02 | 1.01 |
| Idh3b | 1.22 | 1.30 | 2.06 |
| Idh3g | 1.02 | −1.06 | −1.02 |
| Mdh1 | 1.07 | 1.08 | 1.41 |
| Mdh1b | −1.23 | −1.20 | −1.00 |
| Mdh2 | 1.10 | 1.01 | 1.15 |
| Polr3h | −1.19 | −1.20 | −1.34 |
| Sdha | 1.17 | 1.13 | 1.43 |
| Sdhb | 1.08 | 1.13 | 1.42 |
| Sdhc | −1.01 | −1.06 | −1.20 |
| Sdhd | 1.16 | 1.02 | 1.23 |
| Sucla2 | 1.01 | −1.01 | 1.05 |
| Suclg1 | 1.08 | −1.07 | 1.00 |
| Suclg2 | −1.03 | −1.00 | −1.25 |

Table 9 shows the modulation of the genes of the fatty acid metabolism pathway (GO:0006631) caused by calorie restriction (CR), resveratrol alone (Res), or the compositions of the present embodiments (LGX).

TABLE 9
Modulation Of The Genes Of The Fatty
Acid Metabolism Pathway (GO: 0006631)

| | Fold Change | | |
|---|---|---|---|
| Gene | CR | Res | LGX |
| 2010111I01Rik | 1.07 | −1.17 | −1.29 |
| Aacs | 1.35 | −1.12 | 1.32 |
| Aasdh | −1.06 | −1.69 | −2.00 |
| Abat | −1.16 | 1.03 | 1.56 |
| Acaa2 | 1.07 | 1.27 | 1.41 |
| Acadl | 1.19 | 1.21 | 1.85 |
| Acadm | −1.02 | −1.10 | −1.02 |
| Acads | 1.08 | 1.20 | 1.40 |
| Acadvl | 1.06 | 1.14 | 1.38 |
| Acot11 | 1.22 | −1.08 | 1.18 |
| Acot12 | −1.09 | −1.16 | 1.59 |
| Acot2 | −1.35 | 2.13 | 1.58 |
| Acot4 | 1.58 | 1.65 | 2.24 |
| Acot5 | 2.89 | 6.34 | 2.39 |
| Acot7 | 1.20 | 1.03 | 1.06 |
| Acot8 | 1.05 | −1.05 | 1.08 |
| Acox1 | −1.06 | 1.06 | −1.03 |
| Acox2 | 1.17 | 1.01 | 2.52 |
| Acox3 | 1.48 | 1.70 | 1.84 |
| Acoxl | −1.85 | −1.39 | −1.08 |
| Acsbg1 | 1.64 | 2.24 | 1.62 |
| Acsbg2 | −1.55 | −1.14 | −1.37 |
| Acsf3 | 1.28 | 1.17 | 1.57 |
| Acsl1 | 1.05 | 1.21 | 1.38 |
| Acsl3 | −1.91 | −2.10 | −1.17 |
| Acsl4 | −1.34 | −1.20 | −1.35 |
| Acsl5 | 1.19 | 1.04 | 1.19 |
| Acsl6 | −1.15 | −1.23 | −1.26 |
| Acsm1 | 1.27 | 1.25 | 1.43 |
| Acsm2 | −1.33 | 1.06 | −1.19 |
| Acsm3 | −1.03 | 1.12 | 2.21 |
| Acsm5 | −1.38 | 1.14 | 1.08 |
| Adipoq | 1.74 | 1.24 | 2.14 |
| Adipor1 | 1.08 | 1.03 | −1.10 |
| Adipor2 | −1.16 | 1.11 | 1.15 |
| Agpat6 | 1.15 | 1.15 | 1.32 |
| Agt | 1.42 | 1.57 | 2.50 |
| Aldh5a1 | 1.25 | 1.15 | 1.17 |
| Alox12 | 1.04 | 1.08 | 1.02 |
| Alox12e | −2.02 | −2.10 | 1.18 |
| Alox15 | 1.12 | 1.10 | 1.43 |
| Alox5 | 1.18 | 1.22 | 1.32 |
| Alox5ap | 1.07 | −1.08 | −1.09 |
| Alox8 | 1.27 | 1.12 | 2.31 |
| Aloxe3 | −1.14 | 1.00 | 1.21 |
| Ankrd23 | 1.06 | 1.23 | −1.09 |
| Apoa2 | 1.24 | 1.29 | 1.34 |
| Baat | 2.33 | 2.78 | 2.42 |
| Brca1 | −1.08 | 1.29 | 1.36 |
| C1qtnf2 | −1.23 | −1.02 | −1.05 |
| Cav1 | 1.14 | 1.18 | 1.45 |
| Ces3 | −1.16 | 1.06 | 1.23 |
| Cpt1a | −1.04 | 1.27 | 1.49 |
| Cpt1b | 1.07 | 1.27 | 1.45 |
| Cpt1c | −1.69 | 1.29 | −1.22 |
| Cpt2 | −1.14 | 1.01 | −1.03 |
| Crat | 1.16 | 1.45 | 1.71 |
| Crot | −1.11 | −1.07 | −1.37 |
| Cryl1 | −1.44 | −1.14 | −1.31 |
| Cyb5 | −1.10 | −1.21 | −1.10 |
| Dci | 1.06 | 1.13 | 1.24 |
| Degs1 | 1.02 | −1.16 | −1.00 |
| Ech1 | 1.00 | 1.06 | 1.30 |
| Echdc2 | 1.06 | 1.19 | 1.32 |
| Echs1 | 1.07 | 1.01 | −1.09 |
| Ehhadh | −1.02 | 1.12 | 1.00 |
| Elovl1 | −1.03 | 1.15 | 1.18 |
| Elovl2 | −1.91 | −1.31 | −1.34 |
| Elovl3 | −1.12 | 1.11 | 1.47 |
| Elovl4 | 1.00 | 1.45 | −1.29 |
| Elovl5 | −1.12 | −1.68 | −1.91 |
| Elovl6 | 2.36 | 1.27 | 2.38 |
| Elovl7 | 1.41 | 1.20 | 1.39 |
| Fa2h | −1.27 | 1.18 | 1.04 |
| Fads1 | 1.19 | 1.27 | 1.28 |
| Fads2 | 1.10 | 1.15 | 1.39 |
| Fads3 | 1.13 | 1.38 | 1.35 |
| Fasn | 2.54 | 1.54 | 3.10 |
| Fcer1a | 1.55 | 1.73 | 1.05 |
| Ggtla1 | −1.03 | 1.15 | 1.20 |
| Gpam | −1.09 | 1.34 | 1.39 |
| Hadh | 1.08 | 1.18 | 1.36 |
| Hadha | 1.15 | 1.27 | 1.43 |
| Hadhb | 1.80 | 1.57 | 1.51 |
| Hao3 | −1.71 | −4.04 | 1.13 |
| Hnf1a | 1.20 | 1.81 | 1.06 |
| Hpgd | 1.17 | −1.14 | −2.59 |
| Hsd17b4 | 1.15 | 1.25 | 1.44 |
| Lcn5 | 1.58 | 1.74 | 2.30 |
| Lta4h | 1.38 | 1.68 | 1.49 |
| Ltc4s | 1.46 | −1.05 | 1.19 |
| Lypla1 | −1.05 | −1.32 | −1.93 |
| Lypla2 | −1.00 | −1.31 | −1.13 |
| Lypla3 | 1.31 | 1.42 | 1.41 |
| Mapk14 | −1.10 | −1.13 | −1.51 |
| Mcat | 1.04 | 1.01 | −1.15 |
| Mecr | −1.04 | 1.09 | 1.21 |
| Mlstd1 | −1.18 | 1.29 | 1.78 |
| Mlstd2 | −1.20 | −1.19 | −1.29 |
| Mlycd | −1.01 | 1.55 | 1.43 |
| Myo5a | 1.21 | 1.09 | 1.09 |
| Ncf1 | −1.16 | 1.07 | −1.25 |
| Ndufab1 | −1.34 | −1.55 | −2.65 |
| Olah | 1.33 | −1.02 | 1.11 |
| Oxsm | −1.02 | 1.04 | −1.29 |
| Pccb | 1.01 | −1.01 | −1.02 |
| Pdpn | −1.14 | −1.04 | 1.22 |
| Pecr | −1.01 | −1.03 | 1.00 |
| Pex13 | −1.05 | −1.03 | −1.04 |
| Pex5 | 1.43 | 1.70 | 2.17 |
| Pex7 | −1.16 | −1.36 | −1.44 |
| Phyh | 1.10 | 1.15 | 1.20 |
| Plp1 | −1.03 | 1.06 | −1.29 |
| Ppara | 1.14 | −1.06 | −1.28 |
| Ppard | 1.20 | 1.09 | 1.59 |
| Prkaa1 | −1.08 | 1.17 | −1.38 |
| Prkaa2 | −1.17 | −1.22 | −1.31 |
| Prkab1 | −1.12 | 1.04 | 1.02 |
| Prkab2 | 1.04 | −1.07 | −1.38 |
| Prkag1 | 1.09 | −1.14 | −1.02 |
| Prkag2 | −1.05 | −1.16 | 1.03 |
| Prkag3 | −1.24 | 1.34 | −1.45 |
| Prkar2b | 1.95 | 2.01 | 2.24 |
| Ptgds | −1.29 | 1.05 | −1.10 |
| Ptgds2 | −1.77 | −1.02 | −1.12 |
| Ptges | −1.05 | −1.30 | −1.38 |
| Ptges2 | 1.20 | 1.04 | −1.03 |
| Ptges3 | −1.08 | −1.03 | −1.07 |
| Ptgis | 1.16 | 1.17 | 1.66 |
| Ptgs1 | 1.18 | 1.26 | 1.37 |
| Ptgs2 | 1.01 | 1.25 | 1.35 |
| Qk | −1.06 | −1.52 | −2.08 |
| Rnpep | 1.13 | −1.07 | −1.36 |
| Scap | 1.34 | 1.37 | 1.70 |
| Scd1 | 2.18 | 1.67 | 3.27 |
| Scd2 | 1.19 | −1.03 | 1.33 |
| Scd3 | −1.37 | 1.36 | −1.01 |
| Scp2 | −1.02 | 1.06 | 1.16 |
| Slc27a1 | 1.06 | 2.03 | 2.42 |
| Slc27a2 | −1.52 | −1.96 | 1.41 |
| Slc27a3 | 1.31 | 1.84 | 1.65 |
| Slc27a4 | 1.23 | 1.25 | 1.20 |
| Slc27a5 | −1.57 | −1.03 | −1.05 |
| Syk | 1.73 | 1.76 | 2.09 |
| Tbxas1 | 1.00 | −1.06 | −1.03 |
| Tnfrsf1a | −1.01 | 1.53 | 1.47 |
| Tnxb | 1.59 | 1.54 | 2.01 |
| Tpi1 | 1.15 | 1.06 | 1.24 |
| Tyrp1 | −1.43 | −1.18 | 1.09 |
| Ucp3 | 1.80 | 2.02 | 2.79 |

A study of the expression of 20,341 genes in cardiac tissue revealed that 2,829 genes exhibited statistically significant differences in expression (P<0.01). Of these, 7% (approximately 189 genes) exhibited altered expression in animals subjected only to calorie reduced diets; 8% (approximately 226 genes) exhibited altered expression in animals receiving only resveratrol; no additional genes exhibited altered expression in animals that received resveratrol and which were subjected to calorie reduced diets. In contrast, 61% of the 20,341 genes (approximately 1,729 genes) exhibited altered expression in animals receiving only compounds of the present embodiments (e.g., Longevinex®); an additional 2% of the genes (approximately 56 genes) exhibited altered expression in animals that had received compounds of the present embodiments (e.g., Longevinex®) and which had been subjected to calorie reduced diets; an additional 21% of the genes (approximately 594 genes) exhibited altered expression in animals that had received compounds of the present embodiments (e.g., Longevinex®) and resveratrol; an additional 1% of the genes (approximately 28 genes) exhibited altered expression in animals that had received compounds of the present embodiments (e.g., Longevinex®), resveratrol and which had been subjected to calorie reduced diets.

The above data demonstrates that compounds of the present embodiments (e.g., Longevinex®) were effective in modulating gene expression in heart tissue to an extent surpassing even that of calorie restriction. Similar effects have been observed in non-heart tissue. A study of the expression of 20,341 genes in brain tissue revealed that 3,572 genes exhibited statistically significant differences in expression (P<0.01). Of these, 124 genes exhibited altered expression in animals subjected only to calorie reduced diets; 424 genes exhibited altered expression in animals receiving only resveratrol; 10 genes exhibited altered expression in animals that received resveratrol and which were subjected to calorie reduced diets. In contrast, 2,560 genes exhibited altered expression in animals receiving only compounds of the present embodiments (e.g., Longevinex®); 19 additional genes exhibited altered expression in animals that had received compounds of the present embodiments (e.g., Longevinex®) and which had been subjected to calorie reduced diets; 430 additional genes exhibited altered expression in animals that had received compounds of the present embodiments (e.g., Longevinex®) and resveratrol; 5 additional genes exhibited altered expression in animals that had received compounds of the present embodiments (e.g., Longevinex®), resveratrol and which had been subjected to calorie reduced diets.

### Example 4

### Model Mechanism of Action of the Compositions of the Present Embodiments

The compounds of the present embodiments were thus found to greatly exceed the modulation of gene expression observed upon calorie restriction and to alter the expression of genes in key pathways of lipid metabolism, glucose metabolism, oxidative phosphorylation, the Kreb's cycle, ATP synthesis and fatty acid beta oxidation. In summary, the compounds of the present embodiments were found to have a greater specific activity than resveratrol alone, both in terms of the number of genes and the number of different biochemical pathways affected. The results are significant since calorie restriction (CR) is considered the unequivocal method of prolonging life in all forms of life. Generally, reduction of 50% of caloric intake doubles the lifespan of any organism. The above-described experiments demonstrate that the compositions of the present embodiments exert a more powerful influence over genome expression than resveratrol or CR, and marks the first time any technology has been shown to exceed the effects of CR. Furthermore, the compositions of the present embodiments were found to influence genome expression at an earlier stage of life than CR (which requires a life-long adherence to a CR diet to differentiate genes).

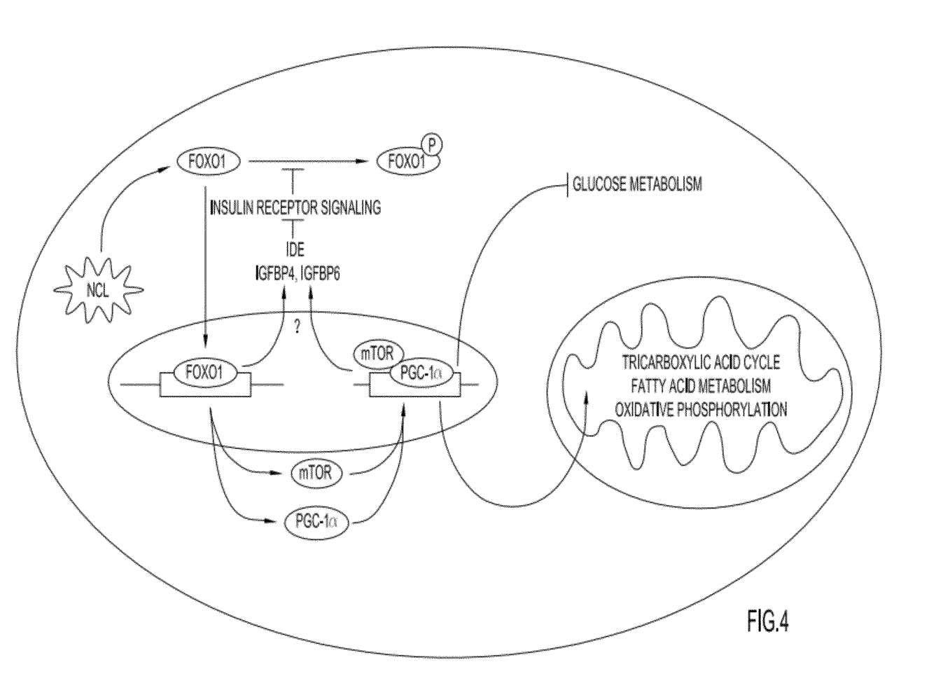

Without intending to be bound by any mechanism of action, the above results suggest that the compounds of the present embodiments act by enhancing the activity of the forkhead Foxo1 (daf-16, dFoxO) transcription factor (FIG. 4). Studies in model organisms have shown that Foxo1 mediates lifespan expression by enhancing gene expression. Insulin/IGF-1 signaling phosphorylates Foxo1, thereby causing it to be excluded from the nucleus and down-regulating its actions. The compounds of the present embodiments decrease insulin and IGF-1 signaling thereby decreasing Foxo1 phosphorylation. Consistent with this model are the observations that the insulin receptor signaling pathway (e.g., GO:008286; genes Ide, Igfbp4, and Igfbp6) is affected by the compounds of the present embodiments. Expression of Foxo1 is increased by 1.75 fold. The compounds of the present embodiments mediate decreased glycolysis and increased gluconeogenesis (e.g., GO:0006006), enhanced Pgc-1α expression (thereby leading to stimulation of Pdk4 expression (e.g., a 1.94 fold increase in Ppargc1α and a 3.25 fold increase in Pdk4), increased expression of lipid metabolism genes (e.g., a 2.79 fold increase in Ucp3, 1.49 fold increase in Cpt1a, and a 1.45 fold increase in Cpt1b). Lipid and fatty acid metabolism genes GO:0006629 and GO:0006635 are uniquely affected by the compounds of the present embodiments. The compounds of the present embodiments thus exert a more pronounced favorable effect on key processes affected by calorie restriction and resveratrol (e.g., chromatin remodeling, transcription from RNA polymerase II promoter, and the ubiquitin cycle. Genes GO:0006333 and GO:0006367 are uniquely affected by the compounds of the present embodiments; Gene GO:0006512 is affected by resveratrol and Longevinex®. Thus, in sum, a proposed mechanism of action is that the compositions of the present embodiments deliver resveratrol to cells, where it passes through cell walls, enters the cytoplasm, and facilitates the translocation of Foxo1 gene into the cell nucleus, which produces the longevity effects.

### Example 5

### Manufacture and Encapsulation of a Composition of the Present Embodiments

Small molecules in the form of resveratrol were obtained via ethanol extraction from vitis vinifera and polygonum cuspidatum. The ethanol was removed, and the resulting extract comprised approximately 25% vinis vinifera skin resveratrol and 25% polygonum cuspidatum resveratrol, with the remainder comprising non-resveratrol, inert plant material. The biological activity of the resveratrol in the extract was confirmed using a SIRT1 Fluorescent Activity Assay/Drug Discovery Kit AK-555 (available from Biomol® Research Laboratories, Inc.; Plymouth Meeting, Pa.; www.biomol.com). The extract was kept in a nitrogen environment and added to a mixture including approximately 25% by weight quercetin; 33% by weight lecithin; and 9% phytic acid (in the form of rice bran extract). The remainder of the composition included approximately 33% by weight resveratrol extract.

The resulting slurry was placed into a capsule-filling machine. Individual dosages were encapsulated in gelatin capsules tinted with titanium oxide (Licaps® capsules available from Capsugel; Greenwood, S.C.; www.capsugel.com). The dosages were encapsulated in a substantially oxygen-free environment using a capsule-filling machine continually flushed with nitrogen (the Capsugel CFS 1000 Capsule Filling and Sealing Machine, available from Capsugel; Greenwood, S.C.; www.capsugel.com). Each resulting capsule included at least 15 mg resveratrol, 100 mg lecithin, 75 mg quercetin, and 25 mg phytic acid. These capsule samples were stored under ambient conditions for approximately eight months. The samples were tested for biological activity by determining whether each sample could activate sirtuin enzymes and, in particular, whether the samples stimulated SIRT1 catalytic activity. The samples were tested four months and eight months after encapsulation. Tests were performed using a SIRT1 Fluorescent Activity Assay/Drug Discovery Kit AK-555 (available from Biomol® Research Laboratories, Inc.; Plymouth Meeting, Pa.; www.biomol.com). Upon testing, it was determined that the resveratrol contained within the samples was biologically active, stimulating SIRT1 activity, producing up to about an eight-fold stimulation in enzymatic activity compared to when no resveratrol is present. Similarly, the biological activity of the quercetin was tested, and it was determined that the encapsulated quercetin maintained biological activity (i.e., the ability to stimulate SIRT1 activity compared to when no quercetin is present).

### Example 6

### Comparative Evaluation of Hormetic Action of Resveratrol and the Present Compositions

Numerous studies have conclusively demonstrated the cardioprotective effects of resveratrol, but one significant fact is often neglected—the hormetic action of resveratrol. Resveratrol is a phytoalexin and many plant-derived products display hormesis. Calabrese et al. (2001) Ann Rev Public Health 22:15-33. Hormesis is defined as a dose-response relationship that is stimulatory at low doses, but detrimental at higher doses resulting in a J-shaped or an inverted U-shaped dose response curve. It has been known for quite some time that cardioprotective effects of alcohol or wine intake follow a J-shaped curve. Constant J (1997) Clin Cardiol 20:420-424. An extensive literature search implicated that resveratrol present in red wine also demonstrates a similar health benefits, being highly effective at lower doses and detrimental at higher doses. The present investigation was undertaken to determine a dose-response curve for resveratrol-mediated cardioprotection and to compare this dose-response curve with another commercially available resveratrol supplement, Longevinex® (Resveratrol Partners LLC, USA). The results of the study revealed that while resveratrol displayed hormetic action, Longevinex® did not.

Animals. All animals used in this study received humane care in compliance with the regulations relating to animals and experiments involving animals, and adheres to principles stated in the Guide for the Care and Use of Laboratory Animals (NIH Publication, 1996 edition), and all the protocols were approved by the Institutional Animal Care Committee of University of Connecticut Health Center, Farmington, Conn., USA. Male Sprague-Dawley rats weighing between 250 and 300 g were fed ad libitum regular rat chow with access to water until the start of the experimental procedure. Animals were randomly subdivided in three groups, of which the control group was gavaged with 1 ml of water containing 5% quartering and 5% hydrate and the other two groups were gavaged with either resveratrol or Longevinex®.

Isolated working heart preparation. After completing the feeding protocol, the animals were anesthetized with sodium pentobarbital (80 mg/kg, intraperitoneally) (Abbott Laboratories, North Chicago, Ill., USA), and heparin sodium (500 U/kg, intravenously) (Elkins-Sinn Inc., Chemy Hill, N.J., USA) was used as an anticoagulant. After deep anesthesia, the hearts were excised, the aorta was cannulated, and the hearts were perfused through the aorta in Langendorff mode at a constant (100 cm of water) perfusion pressure at 37° C. with Krebs-Henseleit bicarbonate for a 5 min washout period as described previously. Ray et al. (1999) Free Rad Biol Med 27:160-9. The perfusion medium consisted of a modified Krebs-Henseleit bicarbonate buffer (millimolar concentration: sodium chloride 118, potassium chloride 4.7, calcium chloride 1.7, sodium bicarbonate 25, potassium dihydrogenphosphate 0.36, magnesium sulfate 1.2 and glucose 10), and after its oxygenization pH was 7.4 at 37° C.

During the washout period, the left atrium was cannulated, and the Langendorff preparation was switched to the working mode for 10 min with a left atrial filling pressure of 17 cm H2O; the aortic afterload pressure was set to 100 cm of water. At the end of 10 min, the baseline cardiac function such as heart rate (HR, beats/min), aortic flow (AF, ml/min), coronary flow (CF, ml/min), left ventricular developed pressure (LVDP, mmHg) and first derivative of developed pressure (LVdp/dt, mmHg/sec) were recorded. Later, 30 min of global ischemia was initiated by clamping the left atrial inflow and aortic outflow lines at a point close to their origins. After 30 min, reperfusion was initiated for 120 min by unclamping the atrial inflow and aortic outflow lines. The first 10 min of reperfusion was in Langendorff mode to avoid ventricular fibrillation, and then the hearts were switched to an anterograde working mode. Ray et al. (1999) Free Rad Biol Med 27:160-9.

Cardiac function assessment. After 10 min of the working mode, baseline parameters were recorded. To monitor the recovery of the heart, the left ventricular cardiac function was recorded after 60 and 120 min of reperfusion. Six rats were present in each group. A calibrated flow-meter (Gilmont Instrument Inc., Barrington, Ill., USA) was used to measure the aortic flow. Coronary flow was measured by timed collection of the coronary effluent dripping from the heart. During the entire experiment, the aortic pressure was monitored using a Gould P23XL pressure transducer (Gould Instrument Systems Inc., Valley View, Ohio, USA) connected to the side arm of the aortic cannula; the signal was amplified using a Gould 6600 series signal conditioner (Gould Instrument Systems Inc.). CORDAT II real-time data acquisition and analysis system (Triton Technologies, San Diego, Calif., USA). Dudley et al. (2009) J Nutr Biochem. 20:443-52. The heart rate, left ventricular developed pressure, and the first derivative of developed pressure were all calculated from the continuously generated pressure signal.

Infarct size estimation. Infarct size was measured using the triphenyl tetrazolium chloride (TTC) staining method. Malik et al. (2006) Antioxidant Redox Signal 8:2101-9. After two hours of reperfusion, 40 ml of 1% (w/v) solution of TTC in phosphate buffer was infused into the aortic cannula, and the heart samples were stored at −70° C. for subsequent analysis. Sections (0.8 mm) of frozen heart were fixed in 2% paraformaldehyde, placed between two cover slips and digitally imaged using a Microtek ScanMaker 600z (Microtek, USA). To quantitate the areas of infarct in pixels, a standard National Institutes of Health image program was used. The infarct size was quantified and expressed in pixels.

Cardiomyocyte apoptosis. Immunochemical detection of apoptotic cells was performed using the terminal deoxynucleotidyl transferase-medicated dUTP nick-end labeling (TUNEL) method. Malik et al. (2006) Antioxidant Redox Signal 8:2101-9. The sections were incubated with mouse monoclonal antibody recognizing cardiac myosin heavy chain to specifically detect apoptotic cardiomyocytes. The fluorescence staining was viewed with a confocal laser microscope. The number of apoptotic cells was counted and expressed as a percent of the total myocyte population.

Statistical analysis. The values for myocardial function parameters, infarct size and apoptosis were expressed as the mean±standard error of mean (SEM). A one-way analysis of variance was performed to test for differences in mean values between groups. If differences were established, the values of the drug-treated groups were compared with those of the drug-free group by modified Student's t-test. The results were considered significant if p<0.05.

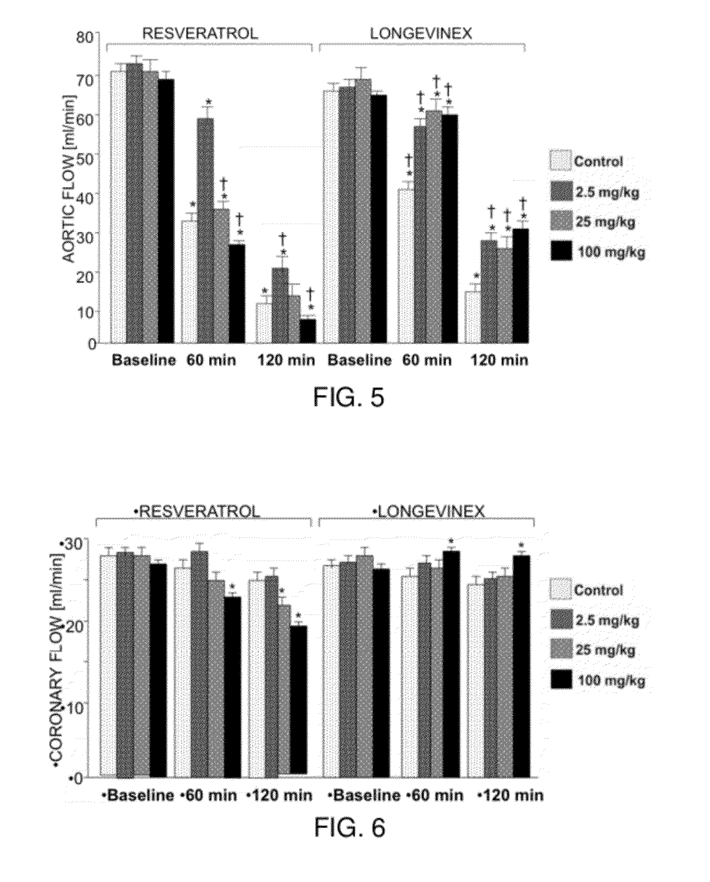

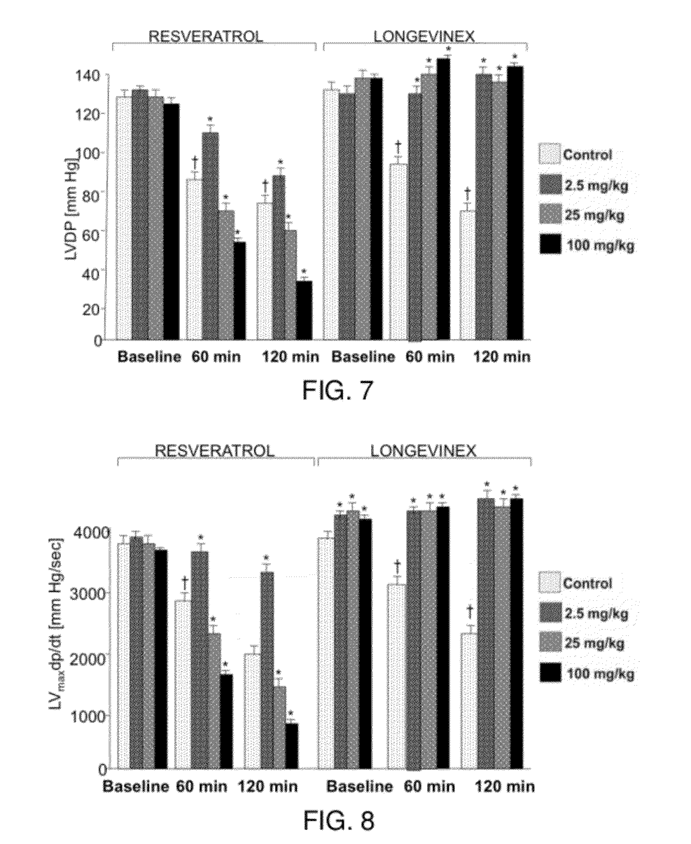

Effects of different doses of resveratrol on cardioprotection. First, the animals were treated with different doses of resveratrol (2.5 mg/kg, 25 mg/kg and 100 mg/kg) by daily gavaging for 21 days. At the end of the 21 days, the animals were sacrificed, their hearts were excised and isolated, and ischemia was induced for 30 min by terminating the coronary flow (as described in the methods section). This was then followed by 2 h of reperfusion in the working mode; during the reperfusion left ventricular function was monitored. As depicted in FIGS. 5 through 8, resveratrol at doses of 2.5 and 25 mg/kg conferred cardioprotection as evidenced by improved aortic flow, left ventricular developed pressure and maximum first derivative of the developed pressure. Above 25 mg/kg, ventricular function was deteriorated as evidenced by significant reduction of aortic flow, LVDP and maximum LVdP/dt. Above 50 mg/kg (data not shown], especially at 100 mg/kg, there was no aortic flow or developed pressure indicating that the hearts ceased functioning.

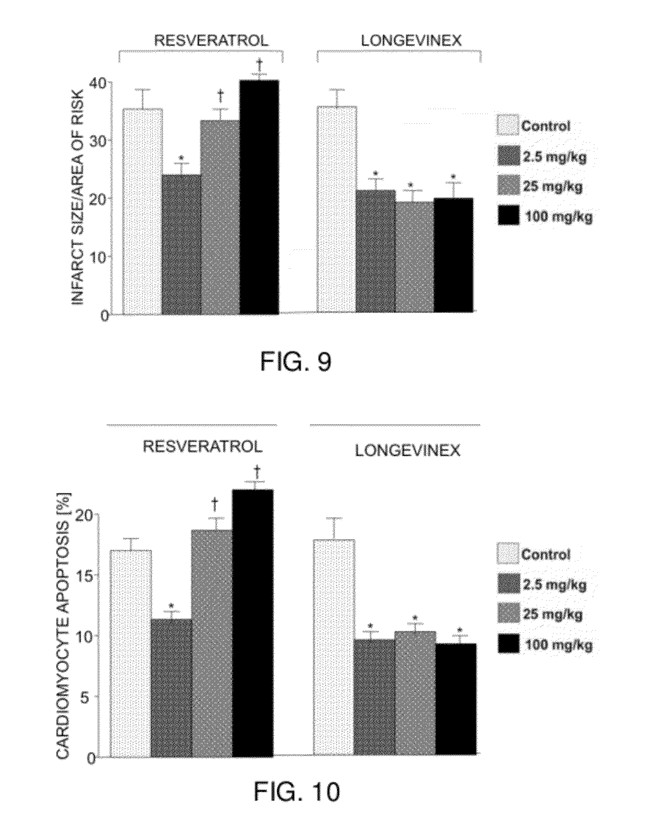

At the end of each experiment the hearts were either subjected to TTC staining to determine infarct size or TUNEL staining to detect apoptosis. The results are shown in FIGS. 9 and 10. Resveratrol significantly reduced the myocardial infarct size and cardiomyocyte apoptosis at doses of 2.5 and 25 mg/kg. However, above 50 mg/kg [data not shown at 50 mg/kg], myocardial infarct size and number of apoptotic cardiomyocytes were significant increased, indicating cellular injury

Effects of different doses of Longevinex® on cardioprotection. A parallel experiment was conducted with Longevinex® by gavaging the rats with three different doses of Longevinex® (2.5 mg/kg, 50 mg/kg and 100 mg/kg) for up to one month. The results are shown in FIGS. 5 through 10. Unlike resveratrol, which showed hormesis, Longevinex® displayed the same degree of cardioprotection up to a dose of 100 mg/kg. It is interesting to note that even at a dose as low as 25 mg/kg, Longevinex® could provide the same degree of cardioprotection as depicted in the results of left ventricular function, LVDP, maximum LVdP/dt as well as infarct size and cardiomyocyte apoptosis. The dose-response curves of resveratrol [J-shaped) and Longevinex® (FIG. 9) clearly demonstrate only pure resveratrol and not Longevinex® displayed hormesis.

Because Longevinex® proved to be cardioprotective over a wide range of concentrations, it was further tested on another animal species. A group of New Zealand white rabbits was gavaged with Longevinex® (100 mg/kg) for 6 months, while the control group was given a placebo. After the completion of the feeding protocol, isolated working rabbit hearts were subjected to 30 min of ischemia followed by 2 h of reperfusion. The results of the infarct size are shown in FIG. 12. Cardiac function remained improved for up to 6 months of Longevinex® feeding (Table 10 below), and infarct size and apoptosis remained lowered for the same duration of time.

TABLE 10
Cardiac Function in Control and Longevinex ®-Treated Working Rabbit Hearts

| 1 month | | 3 months | | 6 months | |
|---|---|---|---|---|---|
| Control | Treated | Control | Treated | Control | Treated |

Preischemic values

| | | | | | | |
|---|---|---|---|---|---|---|
| HR | 234 ± 10 | 229 ± 10 | 226 ± 11 | 231 ± 11 | 229 ± 9 | 231 ± 8 |
| CF | 68 ± 6 | 66 ± 6 | 71 ± 6 | 70 ± 6 | 69 ± 8 | 63 ± 7 |
| AF | 94 ± 8 | 95 ± 8 | 85 ± 7 | 87 ± 7 | 86 ± 8 | 88 ± 7 |
| LVDP | 138 ± 10 | 139 ± 10 | 130 ± 9 | 121 ± 10 | 131 ± 12 | 136 ± 8 |

After 60 min reperfusion

| | | | | | | |
|---|---|---|---|---|---|---|
| HR | 217 ± 9 | 223 ± 9 | 222 ± 9 | 218 ± 9 | 214 ± 9 | 218 ± 12 |
| CF | 48 ± 6 | 55 ± 4 | 55 ± 6 | 66 ± 5* | 52 ± 6 | 63 ± 4* |
| AF | 41 ± 9 | 43 ± 8 | 42 ± 9 | 58 ± 6* | 38 ± 8 | 47 + 4* |
| LVDP | 87 ± 8 | 91 ± 6 | 91 ± 7 | 102 ± 7 | 75 ± 7 | 85 ± 7* |

After 120 min reperfusion

| | | | | | | |
|---|---|---|---|---|---|---|
| HR | 199 ± 10 | 195 ± 9 | 199 ± 9 | 206 ± 10 | 200 ± 9 | 204 ± 12 |
| CF | 42 ± 5 | 44 ± 5 | 47 ± 5 | 58 ± 6 | 48 ± 5 | 60 ± 7* |
| AF | 21 ± 6 | 28 ± 7 | 23 ± 7 | 39 ± 5* | 17 ± 5 | 33 ± 6* |
| LVDP | 62 ± 5 | 65 ± 7 | 64 ± 6 | 77 ± 5* | 56 ± 5 | 69 ± 8* |

Data are presented as mean ± SEM (six rabbits per group).
*P < 0.05 compared with the values of the control ischemia (IS)/reperfusion (RE) group. HR = heart rate (beats/min); CF = coronary flow (ml/min); AF = aortic flow (ml/min); LVDP = left ventricular developed pressure (mm Hg).

The results of the present study clearly demonstrate that resveratrol is beneficial to the heart only at low doses, and is detrimental at higher doses. Also, the action of resveratrol is quickly realized, in most cases within 14 days to 30 days; prolonged resveratrol use does not add any additional benefit. However, we did not study whether prolonged use of resveratrol could cause any adverse effects. Such hormetic effects have been known for more than 100 years, and frequently observed among toxins. Resveratrol is a phytoalexin, whose growth is stimulated by environmental stress such as fungal infection, UV radiation and water deprivation. Adrian et al. (1996) J Agri Food Chem 44:1979-81.

The cardioprotective effects of resveratrol are exerted through its ability to precondition a heart, which causes the development of intracellular stress leading to the upregulation of intracellular defense system such as antioxidants and heat shock proteins. Wallerath et al. (2002) Circulation 106:1652-1658. Preconditioning is another example of hormesis, which is potentiated by subjecting an organ (such as the heart) to cyclic episodes of short durations of ischemia, each followed by another short duration of reperfusion. Das et al. (2003) Arch Biochem Biophys. 420:305-311. Such small but therapeutic amounts of stress render the heart resistant to subsequent lethal ischemic injury. Such an adaptive response is commonly observed with aging. Consistent with this idea, resveratrol has been found to stimulate longevity genes, and at least in prokaryotic species extends the life span. Mukherjee et al. (2009) Free Rad Biol Med 46:573-578; Wood et al. (2008) Nature 7:63-78. In this respect, resveratrol may fulfill the definition of a hormetin. Rattan (2008) Aging Res Rev 7:63-78.

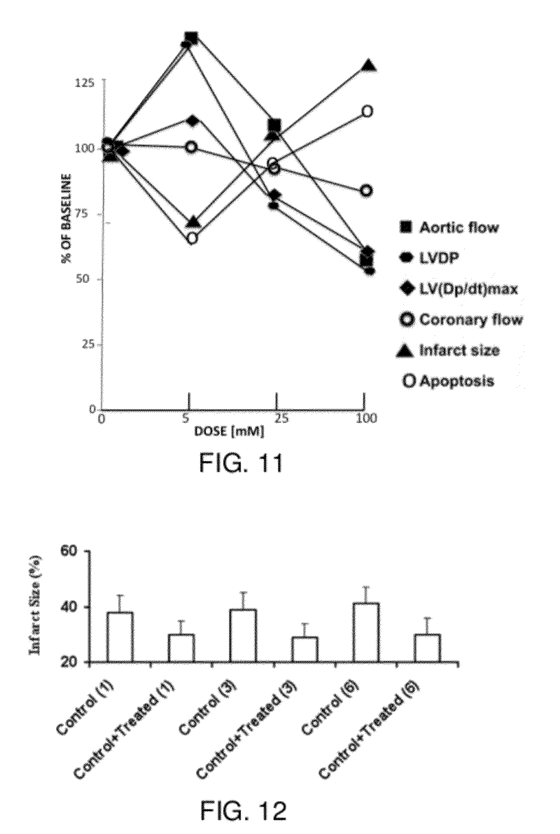

There is no doubt that alcohol, wine, and wine-derived resveratrol all display hormesis. It is known that cardioprotective effects of alcohol or wine intake follow a J-shaped curve, Calabrese et al. (2001) Ann Rev Public Health 22:15-33 and Constant J (1997) Clin Cardiol 20:420-424, and the present study echoed this finding (FIG. 11). At lower doses, resveratrol acts as an anti-apoptotic agent, providing cardioprotection as evidenced by increased expression in cell survival proteins, improved post-ischemic ventricular recovery and reduction of myocardial infarct size and cardiomyocyte apoptosis by maintaining a stable redox environment compared with control. At higher doses, however, resveratrol depresses cardiac function, elevates levels of apoptotic protein expressions, results in an unstable redox environment, and increases myocardial infarct size and the number of apoptotic cells. A significant number of reports are available in the literature to show that at a high dose, resveratrol not only hinders tumor growth but also inhibits the synthesis of RNA, DNA and protein; causes structural chromosome aberrations, chromatin breaks, chromatin exchanges, weak aneuploidy, higher S-phase arrest; blocks cell proliferation; decreases wound healing, endothelial cell growth by fibroblast growth factor-2 (FGF-2) and vascular endothelial growth factor (VEGF); and inhibits angiogenesis in healthy tissue cells leading to cell death. Dudley et al. (2009) J Nutr Biochem. 20:443-52.

Longevinex® was tested side-by-side to the pure resveratrol. Longevinex® did not show any hormetic action (cytotoxicity) up to a dose of 100 mg/kg. It should be noted that any dose of pure resveratrol over 50 mg/100 g stops the heart. Dudley et al. (2009) J Nutr Biochem. 20:443-52. We also determined the long-term effect of Longevinex® on different species of animals, e.g., rabbits, and found that even after 6 months of treatment, Longevinex® provided cardioprotection. The results found in the present study are important for scientists, clinicians and the nonmedical community because it highlights the importance of using resveratrol alone only at lower doses because harmful or toxic effects can occur at higher doses, resulting in adverse effects on health. Longevinex®, however, did not exhibit harmful or toxic side effects at low or high doses. Epidemiological and clinical trials need to be based on the clear understanding of hormetic beneficial effects of resveratrol.

### Example 7

### Restoration of Altered MicroRNA Expression in the Ischemic Heart with Compositions of the Present Embodiments

As reported by Mukhopadhyay P, Mukherjee S, Ahsan K, Bagchi A, Pacher P, and Das D, cardiprotection by resveratrol and its derivative in ischemia/reperfusion [I/R] rat model was examined with miRNA expression profile. Mukhopadhyay et al. (2010) Restoration of Altered MicroRNA Expression in the Ischemic Heart with Resveratrol. PLoS ONE 5 (12): e15705. doi:10.1371/journal.pone.0015705. A unique expression pattern were found for each sample, particularly with Longevinex®, a commercially available resveratrol supplement of the present embodiments available from Resveratrol Partners LLC, USA. Longevinex® and resveratrol pretreatment modulated the expression pattern of miRNAs close to the control level based on PCA analyses. Differential expression was observed in over 50 miRNAs, some of them, such as mir21 were previously implicated in cardiac remodeling. The target genes for the differentially expressed miRNA include genes of various molecular function such as metal ion binding, sodium-potassium ion, transcription factors, which may play a key role in restricting the damage in the heart.

As discussed below in more detail, Longevinex® in particular exerted a much greater influence over microRNAs miR-539 and miR-20b than plain resveratrol. These two microRNAs control VEGF and HIF-1 genes involved in neovascularization, suggesting therapeutic applications with respect to wet macular degeneration, any neovascular eye disease (glaucoma), diabetic retinopathy, and in particular, cancer.

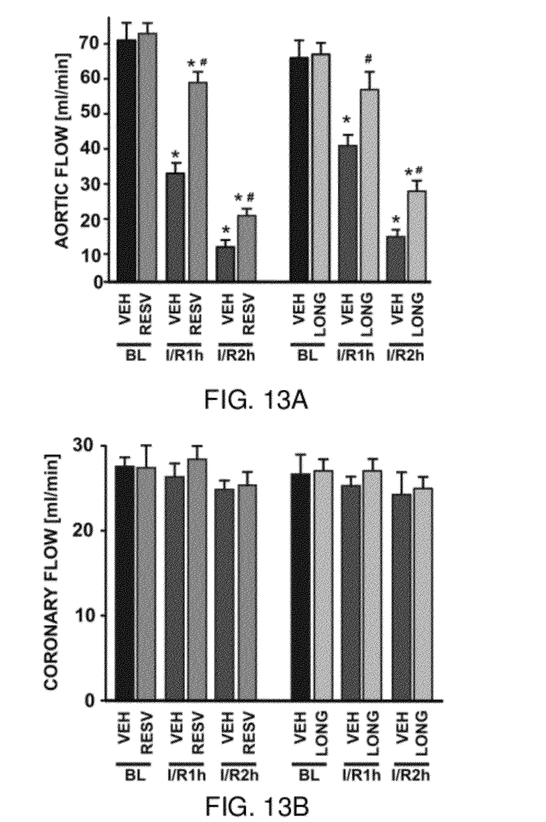

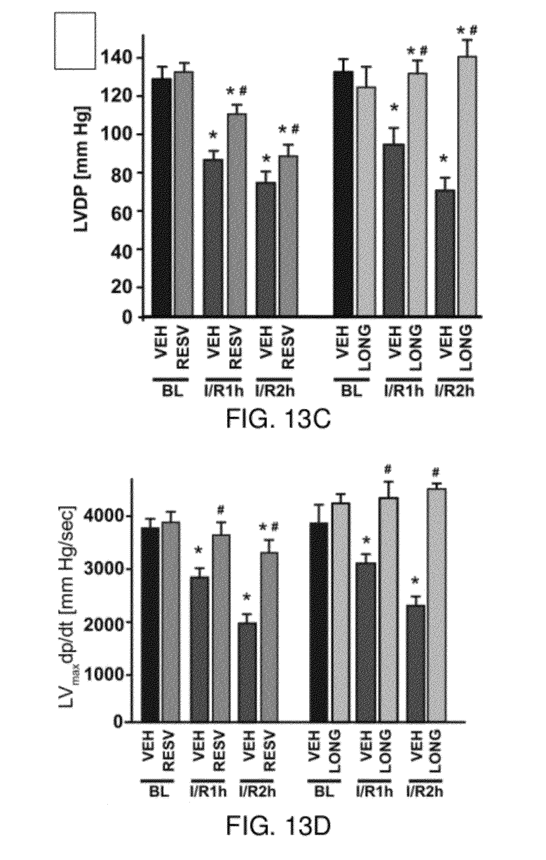

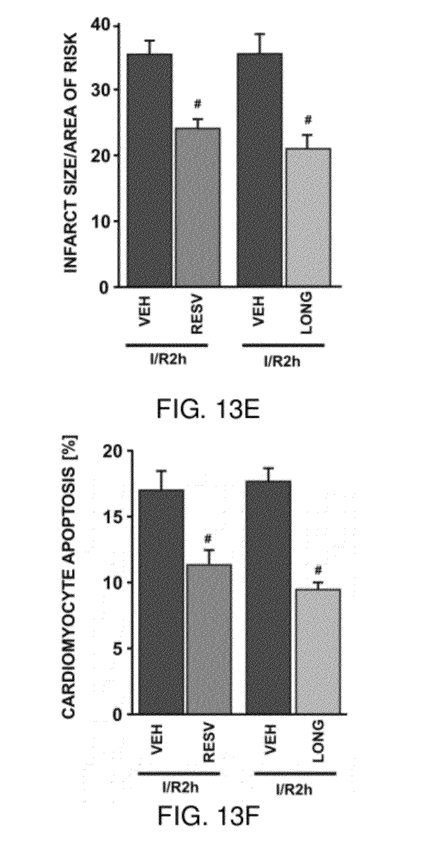

Results and Discussion: Resveratrol and Longevinex® improve cardiac function and reduce myocardial infarct size and cardiomyocyte apoptosis in the IR rat heart. In accordance with previous studies, both resveratrol and Longevinex® improved cardiac output function including aortic flow, coronary flow, left ventricular developed pressure (LVDP) and its first derivative LVₘₐₓ dp/dt 2 hour of reperfusion period (FIG. 13). Gurusamy et al., Cardiovasc. Res. 86:103-112 (2010). FIG. 13 depicts the effects of resveratrol and Longevinex on aortic blood flow (FIG. 13A), coronary flow (FIG. 13B), LVDP (FIG. 13C), dp/dtₘₐₓ (FIG. 13D), infarct size (FIG. 13E), and apoptosis (FIG. 13F). Coronary flow, aortic flow and LVDP were estimated at baseline and at the indicated times of reperfusion. Infarct size and apoptosis were measured at the end of two hours of reperfusion. Results are expressed as Means plus/minus SEM of six animals per group. *p<0.05 vs. Vehicle (VEH). # p<0.05 vs corresponding I/R. BL: Baseline; I/R1h: Ischemia for 30 min and 1 h reperfusion; I/R2h: Ischemia for 30 min and 2 h reperfusion; RESV: Resveratrol; LONG: Longevinex®.

These compounds also lowered the infarct size and death due to cardiomyocyte apoptosis, as expected. A significant number of studies exist in the literature demonstrating cardioprotective role of resveratrol. Recent studies also showed that commercially available resveratrol formulation Longevinex® was equally cardioprotective. We compared the effects of resveratrol with Longevinex®, because recent studies determined Longevinex® to be equally cardioprotective without exhibiting hormetic action of resveratrol, and found that the cardioprotective effects of resveratrol and Longevinex® were consistent with the previously published reports by Mukherjee et al., J. Exp. Clin. Cardiology (in press).

Global miRNA expression profiling in ischemia-reperfused rat heart. MicroRNA profiles were analyzed by TLDA array specific for 586 miRNA and five endogeneous control for rat. Array were carried out in six different groups namely basal level (BL): (1) control vehicle, (2) Resveratrol, (3) Longevinex®, and ischemic repurfused (IR): (4) control vehicle I/R, (5) pretreated (21 days) with Resveratrol I/R and (6) pretreated (21 days) with Longevinex® I/R. RNAs were isolated after 30 min ischemia and 2 hour reperfusion of the heart from IR samples or from baseline (BL) samples processed the same way without ischemia and reperfusion.

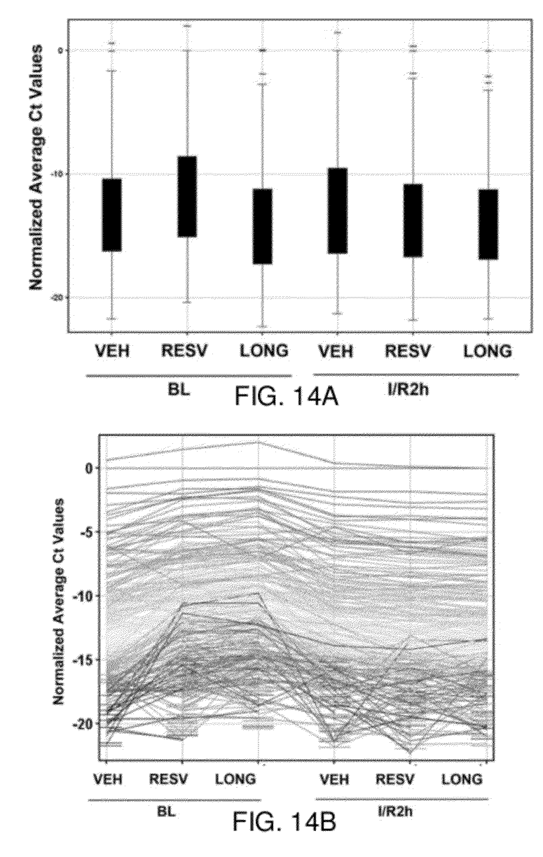

FIG. 14A is a box Whisker plot demonstrating unique distribution of total miRNA expression for all samples. The box whisker plot shows the median in the middle of the box, the 25th percentile (lower quartile) and the 75th percentile (the upper quartile). The whiskers are extensions of the box, snapped to the point within 1.5 times the interquartile. The points outside the whiskers are plotted as they are and considered the outliers and excluded for analysis. The data (Ct values) were normalized based on endogenous genes. Few miRNA were observed to be outliers and 385 miRNA out of 586 were observed to be expressed at least in one of the six conditions. FIG. 14B is a profile plot showing expression of 385 miRNA after normalization to endogeneous control for each samples. miRNA expression were further analyzed by transforming to “fold change” compared to basal level control sample. BL: Baseline; I/R2h: Ischemia for 30 min and 2 h reperfusion; VEH: Vehicle, RESV: Resveratrol; LONG: Longevinex®.

Expression of 213 miRNA were expressed at least 2 fold or higher under one of six conditions. The list was further filtered after looking into miRNA which were either up or down 2-fold in IR samples. Top 25 miRNA were listed, which were either up or down regulated in IR condition (Table 11) and the most regulations were reversed by pretreatment with resveratrol and Longevinex®. IR samples pretreated with resveratrol and Longevinex® both reversed the up or down regulation in IR Control in the opposite direction in 11 of the 25 miRNAs listed in Table 11. Either resveratrol or Longevinex®, but not both, reversed the up or down regulation compared to IR control in 5 instances. In rest of 9 miRNAs expression were attenuated by either or both.

TABLE 11
Differential Expression Of MicroRNA Expressed In Fold Change
With Respect To Basal Level Control Heart Sample

| miRNA | BL Resveratrol | BL Longevinex ® | IR Control | IR Resveratrol | IR Longevinex ® |
|---|---|---|---|---|---|
| miR-539 | up 1272.9 | up 642.7 | up 214.3 | up 172.4 | up 314.6 |
| miR-27a | up 2.2 | up 2.1 | up 9.3 | up 5.5 | up 1.4 |
| miR-101a | up 28.4 | up 39.2 | up 6.1 | up 3.1 | up 3.3 |
| miR-9 | up 2.6 | up 1.1 | up 5.4 | down 1.7 | down 1.1 |
| miR-667 | up 8.2 | up 6.3 | up 4.4 | up 2 | up 1.2 |
| miR-339-5p | up 13.6 | up 20.7 | up 4.1 | down 1.4 | down 3.8 |
| rno-miR-345-3p | up 40.8 | up 23.1 | up 3.7 | down 12 | down 1.1 |
| miR-10a | up 6.4 | up 5.2 | up 3.5 | down 116 | down 1.6 |
| snoRNA202 | up 3.8 | up 4.7 | up 3.2 | down 6 | down 3 |
| miR-27b | down 1.4 | up 1.9 | up 3.2 | up 1 | up 1 |
| miR-29c | up 5.4 | up 4.5 | up 3.1 | up 1.5 | down 1.5 |
| miR-345-5p | up 14.3 | up 31.7 | up 2.4 | down 4.7 | up 1.1 |
| rno-miR-24-1 | down 25.3 | up 1.2 | up 2.1 | down 1.2 | down 1.9 |
| miR-687 | up 3.8 | up 1.8 | up 2 | down 1.7 | down 11.5 |
| miR-27a | up 34 | up 12.8 | up 1.6 | down 1.7 | up 1.5 |
| miR-31 | up 2.4 | up 1.1 | up 1.6 | down 17.5 | down 2.1 |
| miR-20b | down 6 | down 38.8 | down 112.9 | down 189 | down 1366 |
| miR-760 | down 2.7 | up 2.5 | down 30.8 | up 1.5 | up 2.2 |
| miR-351 | up 3.9 | up 9.1 | down 20.9 | down 1.3 | up 1.9 |
| miR-181c | up 5.3 | up 4.2 | down 6.7 | up 1.4 | down 9.1 |
| miR-21 | up 391.4 | up 760.9 | down 4 | up 61.5 | up 59.3 |
| miR-25 | up 25 | up 11.5 | down 1.9 | up 1.1 | up 4.2 |
| rno-miR-450a | up 4.8 | up 2.4 | down 1.7 | down 1.5 | down 5.4 |
| miR-214 | up 4.2 | up 6.2 | down 1.3 | down 3.9 | down 6.5 |
| miR-324-3p | up 4.9 | up 6.5 | down 1.2 | down 5.6 | down 5.3 |

Longevinex® exceeded the effect of resveratrol in 15 of the 25 miRNAs including miR-10a, miR-20b, miR-21. However, in few miRNAs such as miR-29c, Longevinex® had an opposing effect to resveratrol and the difference may be due to many possibilities including presence of other ingredients in Longevinex®, bio-availability of resveratrol etc. There was a tremendous upregulation of miR-21 expression in basal level controls with resveratrol (up 391.4) and Longevinex® (760.9) which was lowered considerably in IR (up 61.5 and 59.3). miR-539 is upregulated to high level (214 fold) in IR samples and was further up-regulated in resveratrol pretreated samples. Similar observations were also found in miR-27a, miR-101, miR-9, miR-667. Similar but less pronounced change were also found in many other miRNAs.

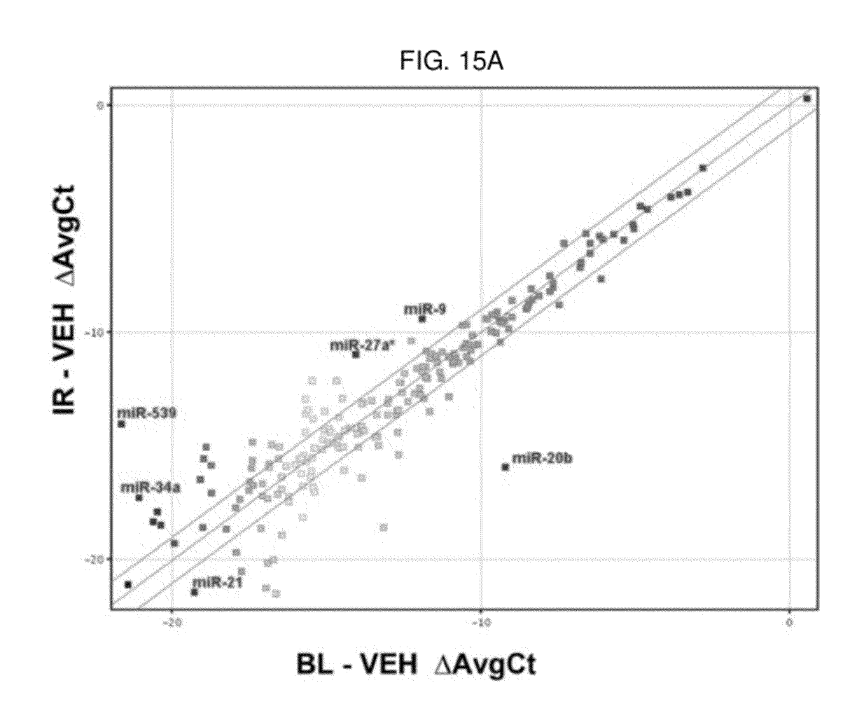

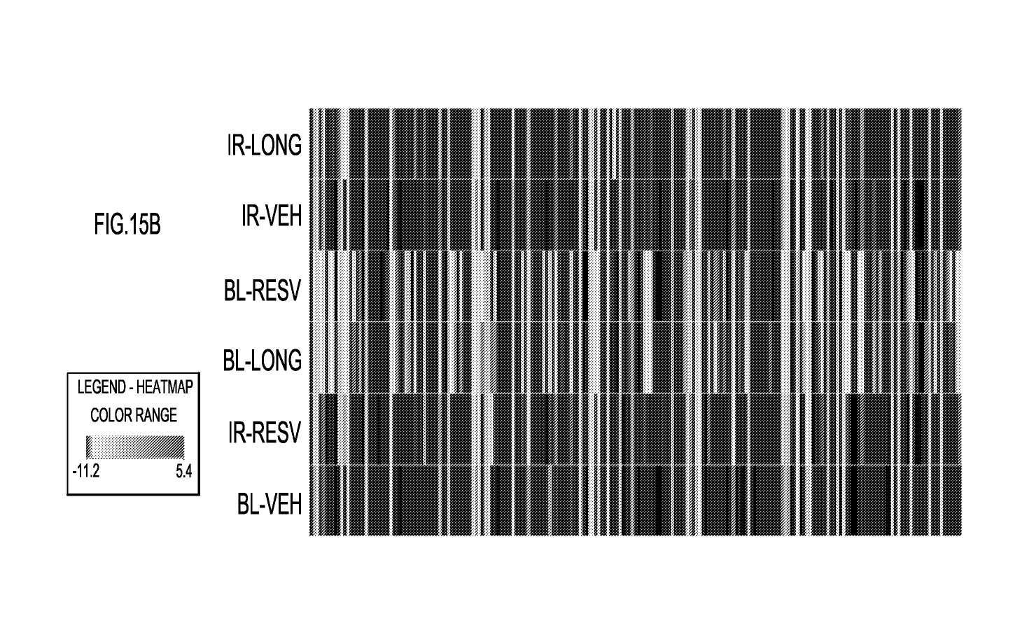

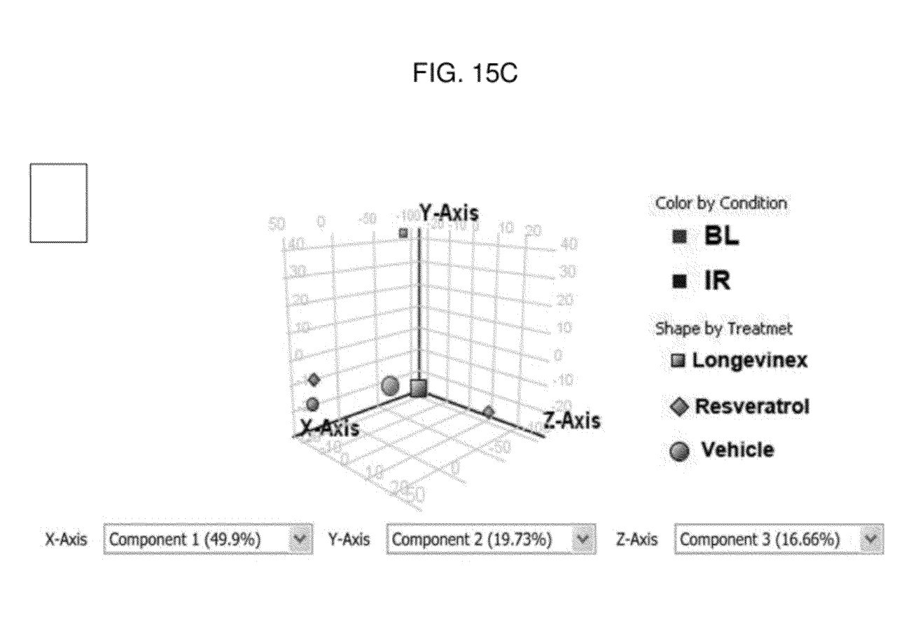

FIG. 15 depicts the effects of resveratrol and Longevinex® on miRNA expression pattern. FIG. 15A depicts the correlation of miRNA expressions between basal level and IR control heart using a scatter plot. Few miRNA expressions were selected for display as shown in Table 11. Double lines indicate as fold change of 2. FIG. 15B depicts a heatmap for cluster analyses of differentially expressed miRNA among samples: Each miRNA was represented as single bar based from their Ct values and color coding was shown below with a gradient from blue (negative and lowest Ct values) to red (positive and highest Ct values). miRNAs not detected were shown as black bars. Each column was represented sample indicated on top. It is evident from the heatmap that treatment with either resveratrol or Longevinex® in control samples altered significant miRNA expression levels, some of them may play significant key roles in cardio-protection. FIG. 15C illustrates principal component analyses of all samples. This multivariate analysis demonstrated the proximity of Longevinex® and resveratrol treated IR samples to the control (vehicle) samples. Principal component analyses of the six samples revealed that the samples IR Longevinex® and IR resveratrol were remarkably similar to BL vehicle sample in terms of gene expression. In the majority of cases, they also were readily distinguished from each group. These results are indeed of utmost importance, as they document that both resveratrol and Longevinex® can protect the ischemic heart by restoring the IR-induced up-regulation or down-regulation of gene expression. BL: Baseline; IR: Ischemia for 30 min and 2 h reperfusion; VEH: Vehicle, RESV: Resveratrol; LONG: Longevinex.

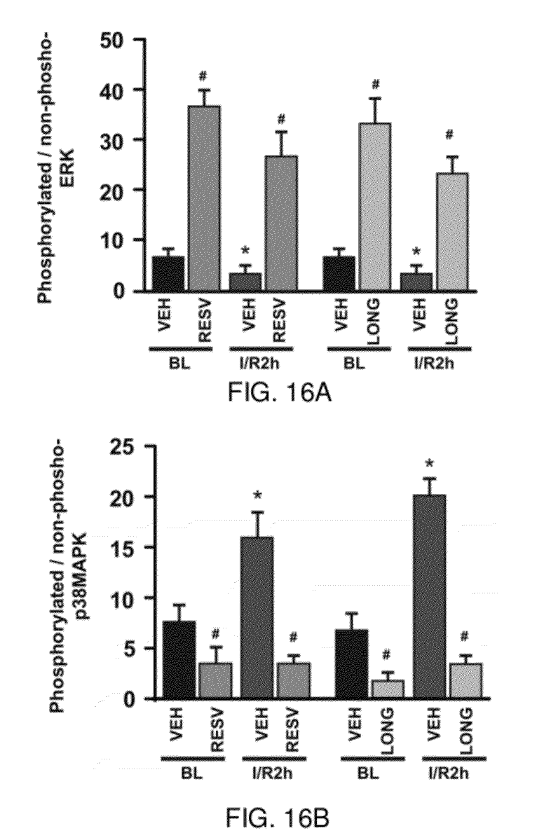

miR-539, the highest upregulated miRNA has 271 conserved gene targets however its functional target has not been reported in the literature. The targets of miR-539 obtained by computational analyses include matrix metallopeptidase 20, fibroblast growth factor 14, clathrin, light polypeptide, osteoprotegerin and transcription factors like forkhead box B1, which may have roles in cardiac remodeling. miR-21 were shown to regulate the ERK-MAP kinase signaling pathway in cardiac fibroblasts, which has role on global cardiac structure and function. Thum et al., Nature 456:980-986 (2008). It has been also shown earlier that resveratrol triggers MAPK signaling pathway as a preconditioning mechanism in heart. Das et al., J. Pharmacol. Exp. Ther. 317:980-988 (2006). We also looked in samples in the ERK-MAPK pathway. As shown in FIG. 16A, ERK phosphorylation was observed to be increased in both resveratrol and Longevinex® treated baseline samples and reduced in corresponding IR samples. In FIG. 16A, the ratio of ERK1/2 phosphorylation to total ERK1/2 were plotted in samples as indicated. A similar but opposing effect was observed in p38 phosphorylation where significantly less phosphorylation occurred in resveratrol or Longevinex® treated BL samples, as depicted in FIG. 16B. Increased p38 MAPK phosphorylation occurred in I/R2h samples and attenuated in both resveratrol and Longevinex® treated I/R2h samples due to preconditioning. In FIG. 16B, the ratio of p38 MAPK phosphorylation to total p38 MAPK were plotted in samples as described. Results are expressed as Means plus/minus SEM of six animals per group. *p<0.05 vs. Vehicle (VEH). # p<0.05 vs corresponding I/R. BL: Baseline; I/R2h: Ischemia for 30 min and 2 h reperfusion; RESV: Resveratrol; LONG: Longevinex®.

VEGF is modulated by miR-20b through HIF1a in cardiomycytes whereas FOXO1 is regulated by miR-27a in cancer cells. Cascio et al., J. Cell Physiol. 224:242-249 (2010); Guttilla et al., J. Biol. Chem. 284:23204-23216 (2009); Tang et al., Cell Death and Differentiation (2008) 15:667-671. SIRT1 were observed to be regulated by miR-9 in stem cells. Saunders et al., Aging (2010). Recent studies demonstrated the increase of miR-1 in coronary artery diseases (CAD) and miR-1 is downregulated by beta-blocker propranolol in rat model of myocardial infarction. Lu et al., Cardiovasc. Res. 84:434-441 (2009). Specific modulations of microRNA by resveratrol have not shown in any in vivo models. Recently microarray analysis of the effect of resveratrol has been demonstrated in human acute monocytic leukemia cell line (THP-1) and human colon adenocarcinoma cell line (SW480). Tili et al. Biochem. Pharmacol. 80:2057-2065 (2010); Tili et al., Carcinogenesis 31:1561-1566 (2010). Resveratrol decreases the levels of miR-155 in THP-1 and modulating JunB and JunD, key regulators in carcinogenesis. Resveratrol also modulates microRNA targeting effectors of TGFbeta pathways. Id. Treatment with resveratrol in cancer cell line SW480 results in decreased level of miR-21 and miR29c whereas it was increased in healthy heart when treated with resveratrol. Id. This anomaly may be due to the fact that cardiomyocytes is barely dividing cells whereas SW480 cells grow rapidly which leads to complete different microenvironment inside cells. It is also important to point out that the doses for resveratrol is much higher (50 micromolar) in cancer cells and similar dose is partially detrimental to human cardiomyocytes and endothelial cells in cultures (data not shown).

Integrative analyses of miRNA for target gene and pathway analyses. Differentially expressed miRNAs were further analyzed for their putative target genes using TargetScan and were listed in Table 12.

TABLE 12
Putative Target Genes for Differentially Expressed miRNA

| Molecular Function Category | Number of Target Genes | Examples of Target Genes |
|---|---|---|
| RNA binding | 101 | Snrpe, Cherp, Phax |
| Actin binding | 40 | Tnni1, Cald1, Cfl1 |
| Signal transducer activity | 10 | Gnb1, Wnt16 |
| Receptor activity | 55 | Gpr155, Mmd2, Gab2 |
| Structural molecule activity | 31 | Lmnb1, Krt1 |
| Calcium ion binding | 109 | Ocm, Calm1, Rad21 |
| Oxidoreductase activity | 52 | Duox2, Aldh2, Gpx7 |
| Phosphatase activity | 51 | Mtmr1, Ptpn1, Styx |
| Potassium ion binding | 50 | Kcnc1, Slc12a4 |
| Sodium ion binding | 54 | Scn4a, Hcn1 |
| Chloride ion binding | 40 | Ano1, Ano1 |
| Sequence-specific DNA binding | 186 | Foxo1, Traf3, Dnmt3b |
| Metal ion binding | 1237 | Dnmt3b, Rarb,Kcnd1 |

Most of the target genes (>1400 genes) have molecular function of metal ion binding, calcium-potassium-chloride ion binding, correlated to the restructuring heart after IR damage. Importantly, miRNA target gene modulated sequence specific DNA factor such as FOXO1, TRAF3 etc. SirT1 regulates several transcription factors including FoxO1, which is inactivated by phosphorylation via Akt. Brunet et al., Science 303:2011-2015 (2004). Recent publication showed the phosphorylation of FoxO1 along with the activation of SirT1, SirT3 and SirT4 are localized in mitochondria where they regulate aging and energy metabolism. Mukherjee et al., Free Radic. Biol. Med. 46:573-578 (2009). Over the years, SIRT1 was known to be activated by resveratrol. Baxter et al., J. Cosmet. Dermatol. 7:2-7. However, resveratrol may have no direct roles in activating SIRT1 Pacholec et al., J. Biol. Chem. 285:8340-8351 (2010). Since dysregulation of miRNAs such as miR-21 is directly linked with cardiac diseases like ischemic heart disease and since resveratrol can ameliorate myocardial ischemic reperfusion injury through the modulation of several miRNAs, the results of the present study explains the mechanism of complex regulatory network mediated by resveratrol through miRNA in cardioprotection.

In summary, microRNA regulate target gene mostly by translational repression and sometimes through translational activation. Here, we demonstrated that resveratrol or Longevinex® regulated miRNA expression in healthy heart and ischemic-reperfused heart. Future detailed studies based on these analyses will pave the way for development of novel therapeutic intervention for cardioprotection in acute I/R injury.

Methods. Animals. All animals used in this study received humane care in compliance with the regulations relating to animals and experiments involving animals and adheres to principles stated in the Guide for the Care and Use of Laboratory Animals, NIH Publication, 1996 edition, and all the protocols (Proposal #2008-484) were approved by the Institutional Animal Care Committee of University of Connecticut Health Center, Farmington, Conn., USA. Male Sprague-Dawley rats weighing between 250 and 300 g were fed ad libitum regular rat chow with free access to water until the start of the experimental procedure. Animals were gavaged with either resveratrol (5 mg/kg/day) [Sigma Chemical Company, St. Louis, Mo.] or Longevinex® (100 mg/kg/day) for 21 days. Previous studies from our laboratory established the appropriate dose and time periods for each compound used in this experiment. Hattori et al., Am. J. Physiol. Heart. Circ. Physiol. 282:H1988-1995 (2002); Mukherjee et al., Can. J. Pharmol. Physiol. 2010 November; 88 (11):1017-25.

Isolated working heart preparation and assessment of cardiac function. After completing the feeding protocol, the animals were anesthetized with sodium pentobarbital (80 mg/kg, i.p.) (Abbott Laboratories, North Chicago, Ill., USA), and intraperitoneal heparin sodium (500 IU/kg, i.v.) (Elkins-Sinn Inc., Chemy Hill, N.J., USA) was used as an anticoagulant. After the deep anesthesia was conformed, hearts were excised, the aorta was cannulated, and the hearts were perfused through the aorta in Langendorff mode at a constant (100 cm of water) perfusion pressure at 37 C with the KHB for a 5 min washout period as described previously. The perfusion medium consisted of a modified Krebs-Henseleit bicarbonate buffer (millimolar concentration: sodium chloride 118, potassium chloride 4.7, calcium chloride 1.7, sodium bicarbonate 25, potassium dihydrogen phosphate 0.36, magnesium sulfate 1.2 and glucose 10), and after its oxygenization pH was 7.4 at 37 C. During the washout period left atria was cannulated, and the Langendorff preparation was switched to the working mode for 10 min with a left atrial 6 filling pressure of 17 cm H₂O, aortic afterload pressure was set to 100 cm of water. At the end of 10 min, baseline cardiac function like heart rate (HR, beats/min), aortic flow (AF, ml/min), coronary flow (CF, ml/min), left ventricular developed pressure (LVDP, mmHg) and first derivative of developed pressure (LVdp/dt, mmHg/sec) were recorded. After that 30 min of global ischemia was initiated by clamping the left atrial inflow and aortic outflow lines at a point close to their origins. At the end of the 30 min of ischemia, reperfusion was initiated for 60 min or 120 min by unclamping the atrial inflow and aortic outflow lines. The first 10 min reperfusion was in Langendorff mode to avoid the ventricular fibrillations, after the hearts were switched to anterograde working mode. Mukherjee et al., Free Radic. Biol. Med. 46:573-578 (2009).

Infarct size estimation. Infarct size was measured according to the TTC method. Mukherjee et al., Free Radic. Biol. Med. 46:573-578 (2009); Imamura et al., Am. J. Physiol. Heart Circ. Physiol. 282:H1996-2003 (2002). After the 2 h of reperfusion, 40 ml of 1% (w/v) solution of triphenyl tetrazolium chloride (TTC) in phosphate buffer was infused into aortic cannula, and the heart samples were stored at −70 C for subsequent analysis. Sections (0.8 mm) of frozen heart were fixed in 2% paraformaldehyde, placed between two cover slips and digitally imaged using a Microtek ScanMaker 600z. To quantitate the areas of infarct in pixels, standard NIH image program was used. The infarct size was quantified and expressed in pixels. Mukherjee et al., Free Radic. Biol. Med. 46:573-578 (2009); Imamura et al., Am. J. Physiol. Heart Circ. Physiol. 282:H1996-2003 (2002).

Assessment of apoptotic cell death. Immunohistochemical detection of apoptotic cells was carried out using the TUNEL method (Promega, Madison, Wis.). Mukherjee et al., Free Radic. Biol. Med. 46:573-578 (2009); Imamura et al., Am. J. Physiol. Heart Circ. Physiol. 282:H1996-2003 (2002). Briefly, after the isolated heart experiments the heart tissues were immediately put in 10% formalin and fixed in an automatic tissue fixing machine. The TUNEL staining was performed according to the manufacturer's instructions. The fluorescence staining was viewed with a fluorescence microscope (AXIOPLAN2 IMAGING, Carl Zeiss Microimaging Inc., New York) at 520620 nm for green fluorescence of fluorescein and at 620 nm for red fluorescence of propidium iodide. The number of apoptotic cells was counted and expressed as a percent of total myocyte population.

Micro RNA isolation and cDNA preparation. Total RNA from rat heart samples were isolated using Trizol reagent (Invitrogen) and further purified using mirVANA miRNA isolation kit (Ambion). Mukhopadhyay et al., Am. J. Physiol. Heart Circ. Physiol. 296:H1466-1483 (2009). cDNAs were prepared using Taqman miRNA Reverse Transcription kit and Megaplex Rodent Pool A and B primers sets.

Profiling of miRNA expression. miRNA expression profiling were carried out using quantitative real-time PCR method by TaqManH Gene Signature Rodent Arrays on a 384 well micro fluidic card in 7900HT Realtime PCR machine (Applied Biosystem, Foster City) according to manufacturer's recommendation. Each miRNA were quantified by two specific amplicon primers and one specific probe. Comprehensive coverage of Sanger miRBase v10 was enabled across a two-card set of TaqManH MicroRNA Low Density Arrays (TLDA Array A and B) for a total of 518, and 303 unique assays, specific to rat miRNAs, respectively. In addition, each array contains six control assays—five carefully selected candidate endogenous control assays, and one negative control assay. Profiling of miRNA by array has been used previously. Chen et al., BMC Genomics 10:407 (2009).

Analyses of miRNA gene expression data. Realtime PCR data expressed as Ct values from array A and B were combined using R script (provided by GeneSpring Informatics Support Team) and processed using GeneSpringGX 11.0.2 software (Agilent Technologies, Santa Clara). After analysis 591 entities were detected from array A and B. All statistical analyses including normalization to endogeneous control, quality control, filtering, correlation analyses and principal component analyses were carried out by GeneSpring GX software.

miRNA Target prediction. miRNA targets have been predicted using TargetScan in-built and plugged within GeneSpring GX software.

Western Blot analysis. Hearts were homogenized in a buffer containing 25 mM Tris-HCl, 25 mM NaCl, 1 mM orthovanadate, 10 mM NaF, 10 mM pyrophosphate, 10 mM okadaic acid, 0.5 mM EDTA, and 1 mM phenylmethylsulfonyl fluoride. One hundred micrograms protein of each heart homogenates separated by SDS-polyacrylamide gel electrophoresis and immobilized on polyvinylidene difluoride membrane. The membrane was immune-blotted with ERK1/2, phospho-ERK1/2, p38 MAPK and phospho-p38 MAPK (Cell signaling Technology, MA) to evaluate the phosphorylation of the compounds. The resulting blots were digitized and subjected to densitometric scanning using a standard NIH image program.

Statistical analysis. The values for myocardial function parameters, infarct size and apoptosis were expressed as the mean±standard error of mean (SEM). A one-way analysis of variance was first carried out to test for any differences in mean values between groups. If differences were established, the values of the resveratrol-treated groups were compared with those of the control group by modified t-test. The results were considered significant if p>0.05.

### Example 8

### Anti-Angiogenic Action in the Ischemic Myocardium with Compositions of the Present Embodiments

As reported by Mukhopadhyay P, Das S, Gorbunov N, Otani H, Pacher P, and Das D, a study was designed to examine the effects of resveratrol and Longevinex® with or without γ-tocotrienol in the ischemic myocardium on hemodynamic functions and angiogenic factors VEGF and HIF-1α. Mukhopadhyay et al., Modulation of MicroRNA 20b with Resveratrol and Longevinex is Linked with Potent Anti-Angiogenic Action in the Ischemic Myocardium: Synergistic Effects of Resveratrol and Gamma-Tocotrienol (in press). Results demonstrated that Longevinex® indeed possesses potent anti-angiogenic action on the heart, which corroborated with its ability to down-regulate VEGF and HIF-1α. Antagomir specific for miRNA 20b reversed the anti-angiogenic action of Longevinex®.

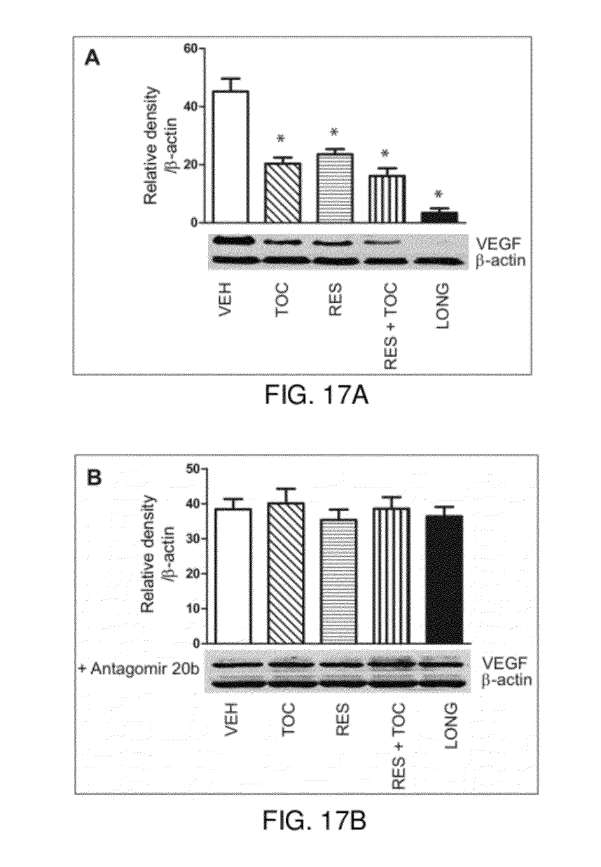

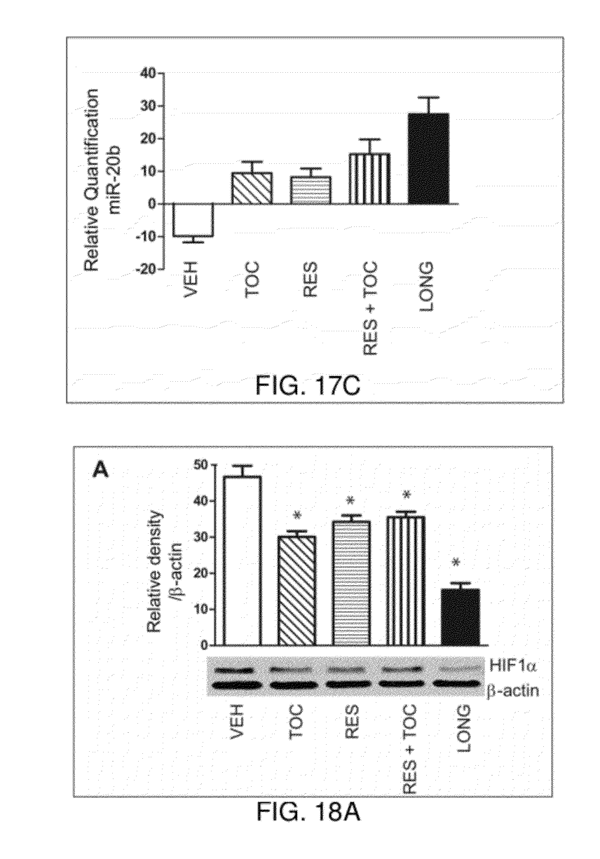

Effects of antagomir-20b on resveratrol, Longevinex® and γ-tocotrienol induced expression of HIF-1α and VEGF. The results for the expression of HIF-1α and VEGF are shown in FIGS. 17 and 18.

FIGS. 17A through 17C are bar graphs (top) quantifying the results of Western blots (bottom) depicting the regulation of miR-20b and the effects of antagomiR-20b on VEGF. FIG. 17A depicts VEGF Western blot analysis and its quantification of the experimental groups are (1) IR sham (vehicle), (2) IR+γ-tocotrienol, (3) IR+resveratrol, (4) IR+γ-tocotrienol+resveratrol, and (5) IR+Longevinex®. * p<0.05 vs IR Sham where n=4/group. FIG. 17B depicts VEGF western blot analyses and its quantification of the same group of samples when pretreated with antagomiR-20b. * p<0.05 vs IR Sham where n=4/group. FIG. 17C depicts Taqman Real-time PCR quantification of the same samples.

FIGS. 18A and 18B are bar graphs (top) quantifying the results of Western blots (bottom) depicting the regulation of miR-20b and the effects of antagomiR-20b on HIF-1a expression. FIG. 18A depicts HIF-1a Western blot analysis and its quantification of the experimental groups (1) IR sham (vehicle), (2) IR+γ-tocotrienol, (3) IR+resveratrol, (4) IR+γ-tocotrienol+resveratrol, and (5) IR+Longevinex®. FIG. 18B depicts HIF-1a Western blot analyses and its quantification of the same group of samples when pretreated with antagomiR-20b. * p<0.05 vs IR Sham where n=4/group.

Animals. All animals used in this study received humane care in compliance with the regulations relating to animals and experiments involving animals and adheres to principles stated in the Guide for the Care and Use of Laboratory Animals, NIH Publication, 1996 edition, and all the protocols (Proposal #2008-484) were approved by the Institutional Animal Care Committee of University of Connecticut Health Center, Farmington, Conn., USA. Male Sprague-Dawley rats weighing between 250 and 300 g were fed ad libitum regular rat chow with free access to water until the start of the experimental procedure. Animals were gavaged with either resveratrol (5 mg/kg/day) [Sigma Chemical Company, St. Louis, Mo.] or Longevinex® (100 mg/kg/day) or γ-tocotrienol [5 mg/kg/day], alone or in combination with resveratrol [5 mg/kg/day] for 21 days. Previous studies from our laboratory established the appropriate dose and time periods for each compound used in this experiment. Hattori et al., Am. J. Physiol. Heart. Circ. Physiol. 282:H1988-1995 (2002); Mukherji et al., Can. J. Pharmol. Physiol. (in press).

Effects of Antagomir miR-20b on the Cardioprotection and the Expression of HIF-1α and VEGF. Because interventions including the treatments with resveratrol, Longevinex® and γ-tocotrienol indicated several-fold upregulation of miRNA 20b, antagomir mirRNA20b was used to specifically examine the role of miRNA 20b on the cardioprotective effects of these compounds. The animals were treated with antagomir miRNA20b [i.v.]. 72 h prior to the experiment. After 72 h, all animals were sacrificed and myocardial function was determined and Western blot analysis was performed. Western blot analysis for HIF-1α and VEGF: The effects of resveratrol, Longevinex® and γ-tocotrienol on the expression of HIF-1α and VEGF were estimated by Western blot analysis using antibodies against VEGF and HIF-1α.

The results shown in FIGS. 17 and 18 indicate that both HIF-1α and VEGF expressions are significantly downregulated after the treatment. For VEGF, when γ-tocotrienol was used in conjunction with resveratrol, there was further reduction of VEGF expression, suggesting synergistic action. Longevinex® resulted in very significant reduction of VEGF expression, far greater than resveratrol and γ-tocotrienol. HIF-1α expression was also reduced with the treatments; however, there were no intergroup differences for reservation and γ-tocotrienol. Again, Longevinex® displayed greater reduction [compared to resveratrol and γ-tocotrienol] of HIF-1α Antagomir miRNA20b restored the expressions of both VEGF and HIF-1α for all the treatments suggesting that expressions of VEGF and HIF-1α are dependent of miRNA 20b.

Modulation of miR-20b in ischemic heart and reversed with resveratrol and γ-tocotrienol. Consistent with the results of Western blots, miR-20b was shown to be modulated drastically in ischemia ischemia-reperfused rat heart. miR-20b significantly down regulated in I/R heart as quantified with Taqman real-time PCR (FIG. 17C). A down regulation (9.8 fold) of mir-20b is reversed to 9.4, 8.2, 15.2 and 27.5 fold in γ-tocotrienol, resveratrol, resveratrol+γ-tocotrienol and Longevinex® pretreated I/R hearts respectively. miR-20b targets HIF1α and modulates VEGFα expression.

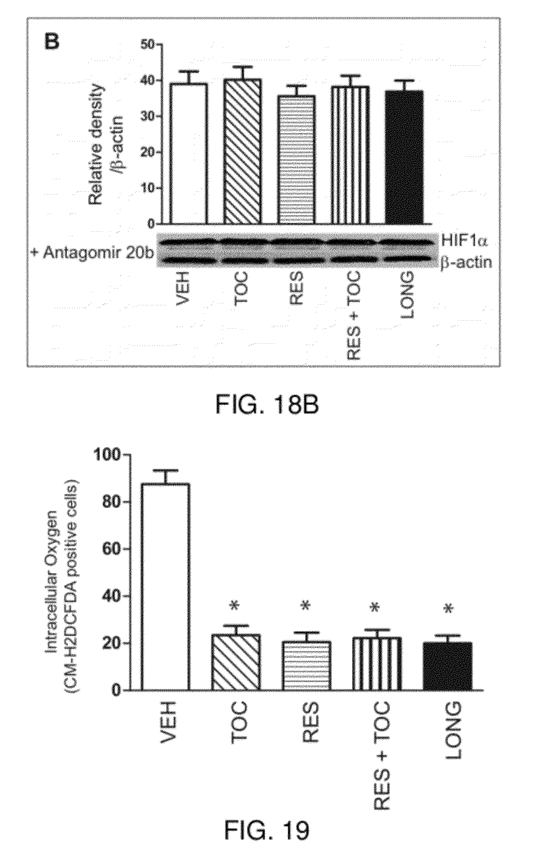

The effects of resveratrol, Longevinex® and γ-tocotrienol on intracellular reactive oxygen species (ROS) activity. Intracellular ROS activity determined by monitoring the level of fluorescence by measuring the fluorescent oxidation product CM-DCF in the cytosol is shown in FIG. 19. FIG. 19 is a bar graph depicting the Intracellular quantification of reactive oxygen species by DCFDA in the experimental groups (1) IR sham (vehicle), (2) IR+γ-tocotrienol, (3) IR+resveratrol, (4) IR+γ-tocotrienol+resveratrol, and (5) IR+Longevinex®. * p<0.05 vs IR Sham where n=4/group. All the compounds including γ-tocotrienol, resveratrol and Longevinex® lowered intracellular ROS concentration compared to control. However, there was no difference between the groups.

Because resveratrol functions by changing ischemia/reperfusion-mediated harmful oxidative environment into a reduced environment, intracellular ROS concentration was determined with CM-H₂DCFDA [5-(and-6)-chloromethyl-2′,7′-dichlorodihydrofluorescein di-acetate, acetyl ester] [10 μM; Molecular Probes, Eugene, Oreg.], a derivative of DCF-DA, with an additional thiol reactive chloromethyl group, which enhances the ability of the compound to bind to intracellular components, thereby prolonging the dye's cellular retention. The dye was injected intravenously, prior to induction of ischemia/reperfusion, and at the end of the experiments, the level of fluorescence was determined for the generation of ROS by measuring the fluorescent oxidation product CM-DCF in the cytosol, at an excitation wavelength of 480 nm and an emission wavelength of 520 nm.

Interestingly enough, the Longevinex® composition showed more potent cardioprotective action and more potent anti-angiogenic effects on heart as evidenced by the down-regulation of VEGF and HIF-1α. The results of resveratrol were compared with the Longevinex® composition, and it was determined that Longevinex® exhibited downregulation of VEGF and HIF-1α, and also showed many-fold induction of microRNA 20-b (a potent anti-angiogenic factor) as compared to that for resveratrol.

All publications and patents mentioned in this specification are herein incorporated by reference to the same extent as if each individual publication or patent application was specifically and individually indicated to be incorporated by reference in its entirety. While the embodiments has been described in connection with specific embodiments thereof, it will be understood that it is capable of further modifications and this application is intended to cover any variations, uses, or adaptations of the embodiments following, in general, the principles of the embodiments and including such departures from the present disclosure as come within known or customary practice within the art to which the embodiments pertains and as may be applied to the essential features hereinbefore set forth.

What is claimed is:

1. A method of modulating a biological activity in a human subject, comprising:
administering to a human subject in need thereof a composition comprising
trans-resveratrol in an amount of 0.25 to 5 mg per kilogram of the human subject,
a metal chelating agent, and
one or more additional antioxidants,
wherein said administration is effective to modulate a biological activity in the human subject as compared to administration of resveratrol alone.

2. The method of claim 1, wherein the metal chelating agent comprises phytic acid.

3. The method of claim 1, wherein the metal chelating agent is present in an amount of 0.25 to 5 mg per kilogram of the human subject.

4. The method of claim 1, wherein the one or more additional antioxidants are present in an amount of 0.05 to 2 mg per kilogram of the human subject.

5. The method of claim 1, wherein the one or more additional antioxidants comprises a phenolic antioxidant.

6. The method of claim 5, wherein the phenolic antioxidant is selected from the group consisting of apigenin, caffeic acid, epigallocatechin 3-gallate (EGCG), ferulic acid, and quercetin.

7. The method of claim 1, wherein the one or more additional antioxidants comprises Vitamin D.

8. The method of claim 1, wherein the composition further comprises one or more glycosaminoglycans selected from the group consisting of hyaluronic acid and chondroitin sulfate.

9. The method of claim 1, wherein the modulation of a biological activity comprises treating or preventing a disease or condition selected from the group consisting of cardiovascular disease, cancer, macular degeneration, a disease associated with aging, a neurodegenerative disease, and inflammation.

10. The method of claim 1, wherein the modulation of a biological activity comprises modulating the expression of a survival or longevity gene.

11. The method of claim 10, wherein the survival or longevity gene is selected from the group consisting of Sirtuin 1, Sirtuin 3, forkhead Foxo1 transcription factor, uncoupling protein 3, or pyruvate dehydrogenase kinase 4.

12. The method of claim 1, wherein the modulation of a biological activity comprises modulating a biological activity selected from the group consisting of oxidative phosphorylation, actin filament length or polymerization, intracellular transport, organelle biogenesis, insulin signaling, glycolysis, gluconeogenesis, and fatty acid metabolism.

13. A method of improving cell transplantation therapy in a human subject, comprising:
co-administering to a human subject transplanted cells and a composition comprising
trans-resveratrol in an amount of 0.25 to 5 mg per kilogram of the human subject,
a metal chelating agent, and
one or more additional antioxidants,
wherein said co-administration is effective to improve the effectiveness of the cell transplantation in the human subject as compared to administration of the transplanted cells alone.

14. The method of claim 13, wherein the co-administration is effective to improve survival of the transplanted cells.

15. The method of claim 13, wherein the co-administration is effective to improve proliferation of the transplanted cells.

16. The method of claim 13, wherein the transplanted cells are selected from the group consisting of cardiac stem cells, neural stem cells, and retinal pigment epithelial (RPE) cells.

17. The method of claim 13, wherein the transplanted cells are cardiac stem cells, and wherein the co-administration is effective to improve differentiation of the cardiac stem cells.

18. A method of improving treatment of macular degeneration in a human subject, comprising:
administering to a human subject suffering from macular degeneration or macular dystrophy a composition comprising
trans-resveratrol in an amount of 0.25 to 5 mg per kilogram of the human subject,
a metal chelating agent, and
one or more additional antioxidants,
wherein said administration is effective to improve the effectiveness of the macular degeneration treatment in the human subject as compared to administration of resveratrol alone.

19. The method of claim 18, wherein the administration is effective to provide one or more benefits selected from the group consisting of improved eyesight, shrinkage of visual defects, and decreased drusen in the eye.

20. The method of claim 18, further comprising co-administering to the human subject the composition and a macular degeneration treatment is selected from the group consisting of an anti-angiogenic medicament and an anti-drusen medicament.
